(19) United States
(12) Patent Application Publication
(10) Pub. No.: US 2020/0316211 A1
(43) Pub. Date: Oct. 8, 2020

(54) LOCALLY ACTING TOLL-LIKE RECEPTOR 7 (TLR7) AND/OR TLR8 AGONIST IMMUNOTHERAPY COMPOUNDS AND THEIR USES

(71) Applicant: Altimmune UK Limited, Leeds (GB)

(72) Inventors: Bertrand Georges, Leeds (GB); Scot Roberts, Gaithersburg, MD (US); Isabelle Peguillet, Gaithersburg, MD (US)

(73) Assignee: Altimmune UK Limited, Leeds (GB)

(21) Appl. No.: 16/837,521

(22) Filed: Apr. 1, 2020

Related U.S. Application Data
(60) Provisional application No. 62/960,380, filed on Jan. 13, 2020.
(60) Provisional application No. 62/827,816, filed on Apr. 1, 2019.

(51) Int. Cl. A61K 47/64; A61K 9/00; A61K 39/00

(52) U.S. Cl. CPC A61K 47/64; A61K 2039/585; A61K 39/0011; A61K 9/0019

(57) ABSTRACT

Provided in the present disclosure are immunotherapy compounds, pharmaceutical compositions thereof and their use, wherein the immunotherapy compounds, upon local administration, form depots inducing cell mediated immune response while mitigating a systemic proinflammatory immune response.

### RELATED APPLICATIONS

This application claims priority to provisional application U.S. Ser. No. 62/827,816 filed on 1 Apr. 2019 and provisional application U.S. Ser. No. 62/960,380 filed 13 Jan. 2020, each of which are hereby incorporated into this application in their entirety.

### SEQUENCE LISTING

The instant application contains a Sequence Listing which has been submitted electronically in ASCII format via EFS-Web and hereby incorporated by reference in its entirety. Said ASCII copy, created on 31 Mar. 2020, is named IPF010US_ST25.TXT and is 2504 bytes in size.

### FIELD OF THE DISCLOSURE

This application pertains generally to an oncology therapeutic compound for use as an intratumoral self-adjuvant to induce a host cell mediated immune response against the tumor.

### BACKGROUND OF THE DISCLOSURE

Intratumoral (IT) or peritumoral (PT) immunotherapy has the potential to stimulate local as well as systemic antitumor immunity. Through direct tumoral delivery, high local concentrations of a therapeutic compound can be achieved while limiting any undesired systemic exposure; that systemic exposure is problematic for certain immunostimulatory compounds that induce non-specific proinflammatory responses such as those leading to a cytokine storm, mimicking symptoms of an acute infection. Specifically, systemic delivery of immunostimulatory compounds exposes healthy tissue to compounds which can either break tolerance inducing autoimmunity or stimulate the immune system inducing cytokine release syndromes.

Hence, immunostimulatory compounds, which find use in oncology applications, have dose-limiting toxicities when administered systemically. However, if the immunostimulatory compounds remain localized, high concentrations and bioavailability of these compounds can be reached at the target area, while the total dose per body weight and systemic exposure is reduced, thus limiting off-target effects. For instance, local delivery of a high concentration of immunostimulatory compounds can be carried out while working with much lower doses than would be needed if the compounds had been administered systemically.

Intratumoral or peritumoral administration of immunostimulatory compounds can convert suppressive and/or protumoral myeloid cells (such as MDSC, TAM and/or M2 cells) into anti-tumor myeloid cells (M1). Reducing local tumor immune suppression can lead to the priming of an immune response against a tumor through the stimulation of infiltrating antitumor B cells, T cells or NK cells. By changing the local tumor microenvironment through this process, an immunologically “cold” tumor can be converted into a “hot” inflamed tumor that may offer better responses to additional immunotherapies, radiotherapies and chemotherapies. In addition, this process may also allow the use of the tumor as its own vaccine by generating antitumor immunity against cancer cell antigens. Upon circulation into the lymphatic and blood vessels, effectors of the antitumor immune response can attack the noninjected, distant, tumor lesions.

For small molecule immune stimulants such as toll-like receptor 7 (TLR7) or TLR8 agonists, intratumoral administration may still lead to a rapid diffusion from the site of administration resulting in toxic effects due to the systemic exposure. Small molecule TLR7 and/or TLR8 agonists are potent immune response modifiers (IRM) that have received considerable attention for the treatment of cancer, viral infections and immune disorders. However, the development of these IRMs has been hampered due to elevated systemic adverse effects as a result of strong cytokine induction (cytokine storm) presumably resulting from rapid systemic diffusion. For example, the TLR7 agonist 852A administered intravenously in patients with melanoma induced severe adverse events in almost 40% of patients (4 out of 13) that completed the first treatment cycle (Dummer, et al. Clin. Cancer Res. 2008, 14(3):856-864). To date, only one TLR7 agonist, imiquimod, has been approved by regulatory agencies for the topical treatment of genital warts, superficial basal cell carcinoma and actinic keratosis. This product is formulated as a 5% imiquimod cream applied on the skin, thus limiting systemic diffusion of the small molecule TLR7 agonist and associated side effects, but also limiting its use because of the topical mode of application.

For cancer treatment, to limit systemic side effects and to create a localized immune response, new drug delivery approaches combined with intratumoral delivery of these agonists are being pursued. Efforts have been made to improve the pharmacokinetic (PK) and pharmacodynamic (PD) properties of TLR7 and/or TLR8 agonists through the development of novel formulations (Dowling, et al. ImmunoHorizons 2018, 2(6): 185-197). A TLR7/8 agonist modified with a C18 lipid moiety (MEDI9197, 3M-052) has been specifically designed for slow dissemination from the site of application. The lipidation of a TLR7/8 agonist formulated in liposomes or oil-in-water (due to its low aqueous solubility) has been demonstrated to reduce systemic pro-inflammatory response while improving the immune response to a co-administered vaccine antigen in animal models (Smirnov, et al. Vaccine 2011, 29(33): 5434-5442). Clinical results assessing intratumoral administration of this compound have demonstrated, even at very low doses (0.005 to 0.055 mg) the induction of adverse events related to cytokine release syndrome leading to the discontinuation of the product development by Medimmune/AstraZeneca (Gupta, S. et al. Cancer Research 2017, 77(13 Supplement): Abstract CT091. Proceedings: AACR Annual Meeting 2017; Apr. 1-5, 2017; Washington, D.C.). Medimmune tried to further improve the delivery of MEDI9197 in combination with a poloxamer 407 thermogel formulation for intratumoral delivery. However, results in animal models indicated that systemic release of the drug after administration of this formulation was still observed that will likely preclude clinical development (Fakhari, et al. J. Pharm. Sci. 2017, 106(8): 2037-2045). Alternatively, to reduce systemic exposure, TLR7 ante-drugs were designed to be rapidly metabolized to a less-active form on entry into the circulation after local application. The ante-drug AZD8848, a TLR7 agonist developed by AstraZeneca for the treatment of asthma, was discontinued due to safety issues resulting from systemic interferon signaling in more than half of the participants resulting in significant influenza-like symptoms (Delaney, et al. BMJ Open Resp. Res. 2016, 3(1): e000113).

Therefore, there remains a need for immunostimulatory compounds that can be administered intratumorally (IT) or peritumorally (PT) and remain localized without inducing a systemic non-specific pro-inflammatory immune response, while also inducing a cell mediated immune response against tumor antigens.

### SUMMARY OF THE DISCLOSURE

Herein are provided immunostimulatory compounds that induce a cell mediated immune response in the absence of a systemic proinflammatory response, pharmaceutical compositions thereof and their use for localized administration.

In certain embodiments are provided immunotherapy compounds having the structure of Formula (I): DM-L-IM. In embodiments, DM (delivery/depot moiety) comprises a peptide from 18 to 45 amino acids in length comprising amino acid residues possessing helix forming properties wherein the DM is configured to form an amphipathic Q-helix structure, and wherein the peptide sequence does not comprise a T cell epitope and/or a B cell epitope relevant to the treated disease and is a non-natural sequence. In embodiments, the DM comprises a peptide of RRLL(5)A(7)LAL(11)A(13)LLRRL (SEQ ID NO. 1). In embodiments, L is a linker covalently attaching the DM component to the IM (immunostimulatory) component. In embodiments IM is a toll-like receptor 7 (TLR7) and/or TLR8 agonist. In embodiments, the DM peptide comprises an amino acid sequence selected from:

(SEQ ID NO: 2)
RRLLHAHLALHAHLLRRLK;
(SEQ ID NO: 3)
RRLLAAHLALHAALLRRLK;
(SEQ ID NO: 4)
RRLLHALLALLAHLLRRLK;
(SEQ ID NO: 6)
KRRLLHALLALLAHLLRRLK;
(SEQ ID NO: 7)
KRRLLAAHLALHAALLRRLK;
or
(SEQ ID NO: 8)
RRLLHALLALLAHLLRRLE.

In certain embodiments, IM is selected from Formula (Ia) to (Im):

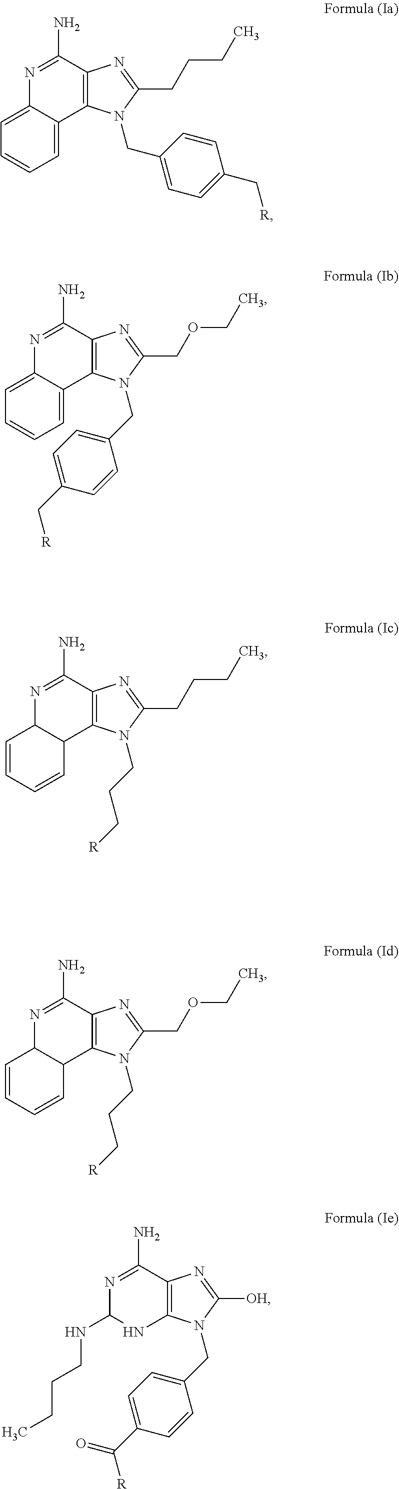

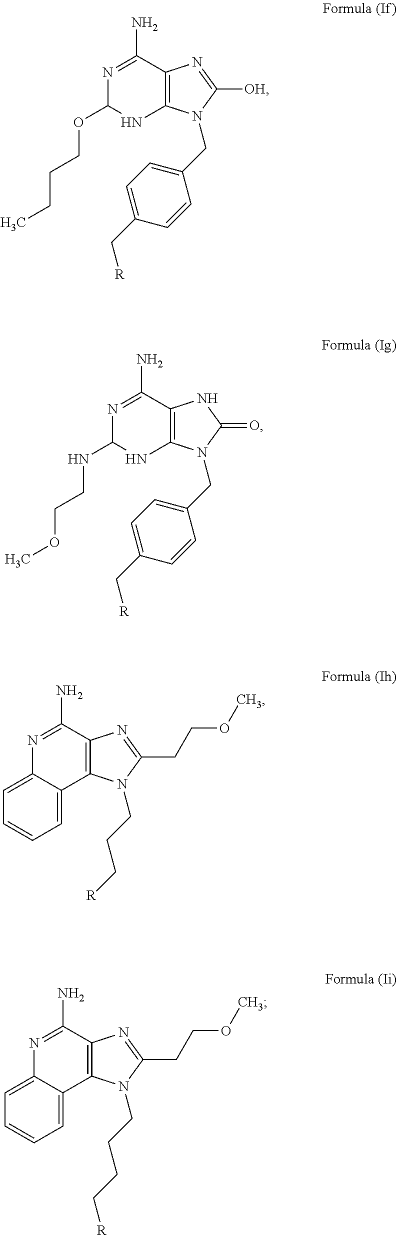

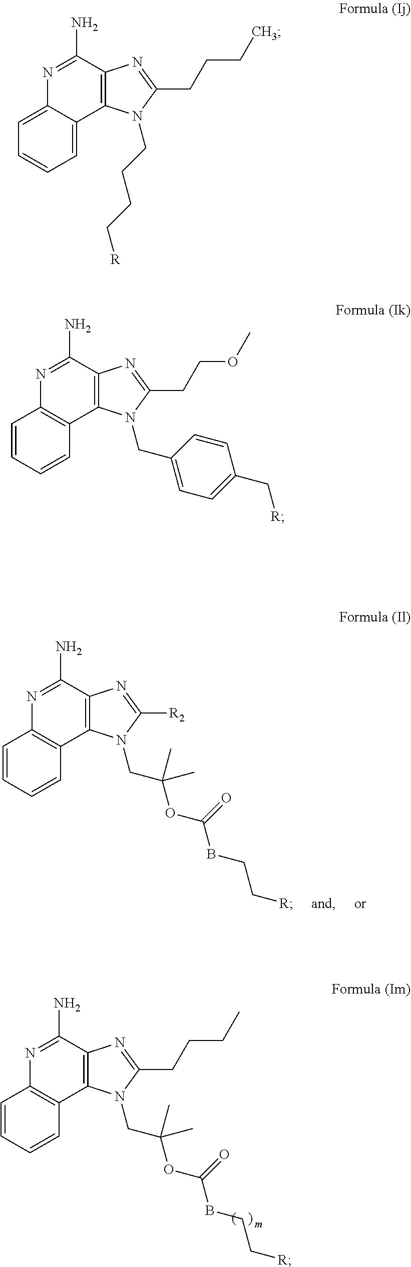

wherein R₂ is selected from:
—CH₃, —CH₂CH₃, —CH₂CH₂CH₃, —CH₂CH(CH₃)₂, —CH₂CH₂CH₂CH₃ (as in, e.g., Formula (Im)) —CH₂CH₂CH₂CH₂CH₃, CH₂OCH₂CH₃, —CH₂CH₂OCH₃, —CH₂NHCH₂CH₃, and —CH₂Ph;
   R comprises the linker (L) connecting the IM to an amino group or carboxyl group of the peptide (DM) at the peptide termini or the lateral chain of an amino acid such as lysine or glutamine, wherein L is -[A1]—NH—, and A1 is selected from:
A2-A3-(CH₂)ₓ—CO—,
A2-A3-CH₂—O—CH₂—CO—,
A2-A3-(CH₂)ₓ—O—(CH₂)ₓ—O—(CH₂)ₓ—O—(CH₂)ₓ—CO—,
A2-Valine-Alanine-A4-,
A2-Valine-Citrulline-A4-,
A2-Glutamate-Valine-Citrulline-A4-, or
A2-Phenylalanine-Lysine-A4-; wherein:
   A2 is selected from:
A5-(CH₂)ₓ-A6-,
A5-(CH₂)ₓ—O—(CH₂)ₓ—O—(CH₂)ₓ—O—(CH₂)ₓ-A3-,
A5-(CH₂)₂-(O—CH₂—CH₂)ₓ-A6-
A5-CH₂—O—CH₂-A6-,
A5-(CH₂)ₓ-A6-,
A5-(CH₂)ₓ—O—(CH₂)ₓ—O—(CH₂)ₓ—O—(CH₂)ₓ-A6-, or,
A5-NH—(CH₂)₂—O—(CH₂)-A6-;

### A3 is —CO— or —NH—;

A4 is p-aminobenzyloxy carbonyl (PABC):

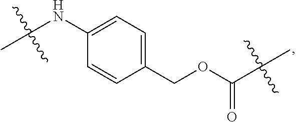 [SMILES: *Nc1ccc(COC(*)=O)cc1]

or A4 is nothing;
A5 and A6 are —CO— or —NH—, one or more natural or non-natural amino-acids, or nothing;

### B is —O— or —NH—;

m is any integer from 1-11;
and,
x is any integer from 1 to 12, and is preferably 2 to 12.

In some embodiments, the peptide conjugate is selected from the group consisting of kHL-12, HL-4X2, AH-3X2, HL-6X2, HL-5X2, HL-4X3, AH-3X3, HL-6X3, HL-5X3, HL-4X4, AH-3X4, HL-6X4, and HL-5X4 and/or, as shown in FIGS. 12-13. In preferred embodiments, the immunotherapy compound is selected from the group consisting of kHL-12, HL-6X2, and HL-6X3.

In embodiments are provided pharmaceutical compositions comprising an immunotherapy compound and a pharmaceutical acceptable carrier or diluent, wherein the immunotherapy compound is soluble in an aqueous solution having a pH range of about 3 to 9 (in embodiments, about 4 to 8) or an ion concentration ranging from 0 mM to 600 mM (in some embodiments about 400 mM).

Also provided herein are methods for inducing a cell mediated immune response using the present immunotherapy compounds. In embodiments, the methods comprise locally administering a liquid form of the pharmaceutical composition into the subject, wherein in vivo physiological conditions reduce solubility of the DM component of the immunotherapy compound wherein the immunotherapy compounds form insoluble self-assemblies or aggregates in vivo; whereby the insoluble self-assemblies or aggregates induce a cell mediated immune response at the local site of administration. In certain embodiments, the immunotherapeutic compound stimulates less systemic proinflammatory cytokines in vivo compared to unconjugated IM component using the same route of administration. In certain embodiments, the methods induce an anti-tumor immune response when administered intratumorally or peritumorally. In other certain embodiments, the methods induce a cell mediated immune response in the mucosa and/or bronchial tissue when administered nasally.

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1 models the change in solubility of the present immunotherapy compounds with a change with pH and/or ion strength of an aqueous solution before and after administration.

FIG. 2 shows Formula I (DM-L-IM) with the representative peptide HL and the alpha-helical structure of the DM component of the present immunotherapy compounds.

FIG. 3 shows a helical wheel representation of peptide HH (A), AH (B), HL (C) and KK (D).

FIG. 4 shows in vivo immunological activity of the oncology therapy compounds HH-12, AH-12 and HL-12 versus the negative control compound KK-12 (and OVA alone).

FIG. 5 shows individual tumor measurements (injected tumors) in groups of animals treated with kHL-12 in combination with anti-PD-1 (“aPD1”) or anti-CTLA-4 (“aCTLA4”) compared to control groups. kHL-12 was administered intratumorally (“IT”) and anti-PD-1, anti-CTLA-4 or vehicle (“PBSlX”) was administered intraperitoneally (“IP”). See Example 5.

FIG. 6 shows individual tumor measurements (non-injected tumors) in groups of animals treated with kHL-12 in combination with anti-PD-1 (“aPD1”) or anti-CTLA-4 (“aCTLA4”) compared to control groups, wherein kHL-12 was administered IT to tumors peripheral to those measured, and anti-PD-1, anti-CTLA-4 or vehicle (“PBSX”) was administered IP. See Example 5.

FIG. 7 shows mean tumor measurements up to day 14 in groups of animals treated with kHL-12 in combination with anti-PD-1 (“aPD1”) or anti-CTLA-4 (“aCTLA4”) compared to control groups. See Example 5.

FIG. 8 shows survival curves for groups of animals treated with kHL-12 via I.T administration in combination with I.P administration of anti-PD-1 (“aPD1”) or anti-CTLA-4 (“aCTLA4”) compared to control groups (“vehicle” and “PBS1X”).

FIG. 9 shows the absence of in vivo toxicity of peptide conjugate AH-L-IM (immunotherapy compound (“AH-12”)) compared to a free TLR7/8 agonist (formula (Ia)-NH₂ (“Free IM”)) administered subcutaneously or Aldara (a commercial product containing imiquimod, a TLR7 agonist) applied topically.

FIG. 10 shows median tumor volume in groups of animals treated with kHL-12 and pHL-12 compared with 3M-052 and R848.

FIG. 11 shows the change in body weight in groups of animals treated with kHL-12 and pHL-12 compared with 3M-052 and R848 as a measure of the respective systemic toxicity of the compounds.

FIG. 12 illustrates the naming/nomenclature and sequence alignment of exemplary peptide conjugates.

FIG. 13 illustrates exemplary peptide conjugates and formulas.

FIG. 14 illustrates Scheme 1 for producing exemplary IM compounds.

FIG. 15 illustrates Scheme 2 for producing exemplary IM compounds.

FIG. 16 illustrates Scheme 3 for producing exemplary IM compounds.

FIG. 17 illustrates Scheme 4 for producing exemplary IM compounds.

FIG. 18 illustrates Scheme 5 for producing exemplary IM compounds.

FIG. 19 illustrates Scheme 6 for producing exemplary IM compounds.

### DETAILED DESCRIPTION OF THE DISCLOSURE

### Introduction

The present invention provides compositions and methods for inducing locally an innate immune response, reducing immune suppressive mechanisms and/or stimulating a cell mediated immune response at the site of administration, while limiting, reducing or avoiding a systemic proinflammatory response and associated adverse events to the compositions.

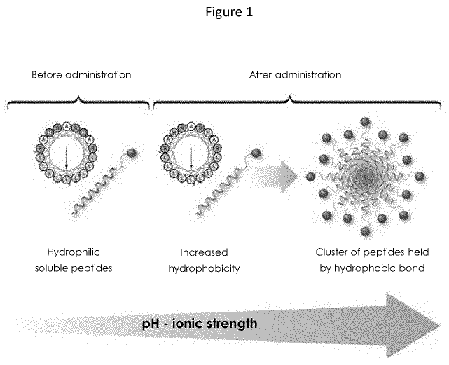

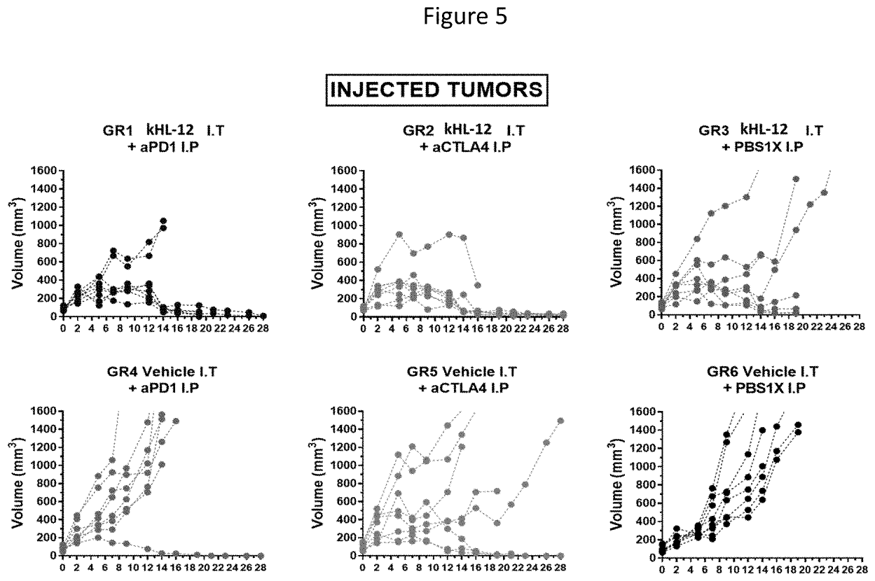

This disclosure provides a peptide-based delivery technology that, when conjugated to a small molecule TLR7 and/or 8 agonist, forms a depot at the administration site to induce a local immune activity while preventing the systemic diffusion of the TLR7 and/or 8 agonist that may otherwise stimulate unwanted proinflammatory cytokine responses. See Examples 5 and 6; and FIG. 1. The present compounds, when administered intratumorally, alone or in combination with checkpoint inhibitors (e.g. anti-CTLA4) further induce an anti-tumor immune response in peripheral or nearby tumors. In other words, wherein the anti-tumor immune response is effective at a distant site from the site of administration of the pharmaceutical composition. See Example 5 and FIGS. 5-8.

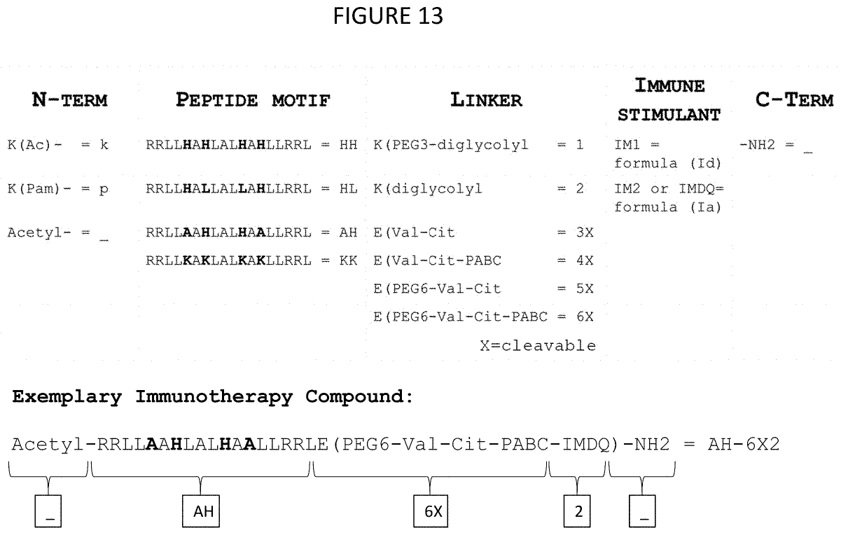

From a pharmaceutical perspective, it is highly advantageous to design a small molecule-peptide conjugate that remains fully soluble during manufacturing, formulation (to allow for sterile filtration) and up to the point of administration. To achieve this, we have designed peptides de novo that have a high propensity to form an amphipathic α-helix structure exposing a hydrophobic face and a hydrophilic face. See FIGS. 1, 2 and 3; and Example 3. Moreover, we have rendered the peptide sensitive to pH and ionic strength by incorporating positively charged residues such as arginine and histidine at specific positions within the peptide sequence. See Example 1, FIG. 13 and Table 1. Histidine residues are advantageous with regard to their pKa near physiological pH. Below physiological pH, histidine residues are likely to present a positive charge and low alpha-helicity. At physiological pH and above, histidine residues are likely to lose their charge, show an increased hydrophobicity and higher alpha-helicity. Based on those principles, we have designed small molecule-peptide conjugates (e.g., immunotherapy compounds) that are demonstrated to be soluble before administration but lose their solubility after administration as a result of their exposure to higher pH and/or ionic strength found in physiological environments. See Examples 4 and 5.

In embodiments provided herein are immunotherapy compounds having the structure of Formula (I): DM-L-IM, wherein DM is a delivery/depot moiety, L is a linker and IM is an immune stimulatory moiety. In embodiments, the DM comprises a peptide from 18 to 45 amino acids in length comprising amino acid residues possessing helix forming properties wherein the DM is configured to form an amphipathic α-helix structure, and wherein the peptide sequence is not derived from an antigen or immunogen and is a non-natural sequence (e.g., wherein the peptide sequence has less than 70% sequence identity with a bacterial, fungal, viral or cancer antigen or immunogen). The linker L covalently attaches the DM to the IM and may be any known linker, including a single covalent bond wherein the linker connects the IM to an amino group or a carboxyl group at the peptide termini or the lateral chain of an amino-acid such as lysine or glutamine of the DM peptide. In embodiments, the IM is a toll-like receptor (TLR) 7 and/or TLR8 agonist. See Examples 1, 2 and 3; and FIGS. 1 to 3.

In embodiments, the linker (L) may be cleavable, consisting of a chemically labile linker including acid-cleavable linkers and reducible linkers or an enzyme cleavable linker such as peptide-based linkers or β-glucuronide linkers well known in the art. In embodiments, the linker L is cleavable via intracellular enzymes (e.g. cathepsin-B). See Example 8 and FIG. 13. In embodiments provided herein are pharmaceutical compositions comprising the present immunotherapy compounds and a pharmaceutically acceptable carrier or diluent. In certain embodiments, the immunotherapy compound is soluble in a pharmaceutical aqueous solution having a combination of pH ranging from of 3 to 9 and an ion concentration ranging from 0 mM to 400 mM. More preferably, the immunotherapeutic compound is soluble in a pharmaceutical aqueous solution having a combination of pH ranging from 6 to 8 and an ion concentration ranging from 0 mM to 300 mM. See Example 3.

In embodiments provided herein are methods for inducing a cell mediated immune response in a subject. In certain embodiments the method comprises locally administering a liquid form of the present pharmaceutical composition into the subject, wherein in vivo physiological conditions reduce solubility of the DM component of the immunotherapy compound wherein the immunotherapy compounds form insoluble self-assemblies or aggregates in vivo; whereby the insoluble self-assemblies or aggregates induce a cell mediated immune response at the local site of administration. See Example 5. Advantageously, the present immunotherapy compounds form a depot and are retained at the site of administration, wherein no to little systemic proinflammatory response is observed. See Example 6 and FIG. 5.

In certain embodiments, the immunotherapy compounds present in a liquid pharmaceutical composition are administered into a tumor (e.g. intratumoral (IT) administration) and induce an innate immune response and a cell mediated immune response against the tumor antigens (e.g. shrink or stabilize the tumor). It is understood the DM comprising a peptide is not an antigen or immunogen, but a mechanism to reduce the solubility of the IM (e.g. TLR7 and/or TLR8 agonist) creating a depot that is retained at the site of administration, such as within a tumor or in the tumor microenvironment. The conjugated IM then stimulates immunosuppressive cells and induces the immune response against the antigens present in the tumor. Moreover, we have found that mobilization of the immunosuppressive cells induces an immune response against not only the tumor at the site of administration, but peripheral, nearby and/or distant tumors as well. See Example 5. In certain embodiments, provided herein are methods of stimulating an anti-tumor immune response in a subject, comprising locally administering intratumorally or peritumorally a liquid form of the pharmaceutical composition into the subject, wherein the anti-tumor immune response is effective at a distant site from the site of administration of the pharmaceutical composition. See Example 5.

In embodiments the use of a peptide (DM) reduces the solubility of an immunostimulant (IM) in extracellular fluid, wherein the peptide is covalently linked to the immunostimulant. In further embodiments are provided an immunotherapy compound for use in a method of treating the human or animal; a pharmaceutical composition comprising an immunotherapy compound of the invention and a pharmaceutically acceptable carrier or diluent; a pharmaceutical composition of the invention for use in a method of treating the human or animal; a pharmaceutical composition of the invention for use in stimulating a cell mediated immune response of an animal or human to a host antigen (e.g. tumor antigen); and the compound of the invention for use in the manufacture of a medicament for stimulating a cell mediated immune response to a host antigen.

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the stated amount plus/minus about 5%, about 4%, about 3%, about 2% or about 1%.

By “administration” is meant introducing a compound of the present disclosure into a subject; it may also refer to the act of providing a composition of the present disclosure to a subject (e.g., by prescribing). The term “therapeutically effective amount” as used herein refers to that amount of the compound being administered which will induce a cell mediated immune response. The term also refers to an amount of the present compounds that will relieve or prevent to some extent one or more of the symptoms of the condition to be treated. In reference to conditions/diseases that can be directly treated with a composition of the disclosure, a therapeutically effective amount refers to that amount which has the effect of preventing the condition/disease from occurring in an animal that may be predisposed to the disease but does not yet experience or exhibit symptoms of the condition/disease (prophylactic treatment), alleviation of symptoms of the condition/disease, diminishment of extent of the condition/disease, stabilization (e.g., not worsening) of the condition/disease, preventing the spread of condition/disease, delaying or slowing of the condition/disease progression, amelioration or palliation of the condition/disease state, and combinations thereof. The term “effective amount” refers to that amount of the compound being administered which will produce a reaction that is distinct from a reaction that would occur in the absence of the compound. In reference to embodiments of the disclosure including the immunotherapy compounds of the disclosure, an “effective amount” is that amount which increases the immunological response in the recipient over the response that would be expected without administration of the compound.

The term “animal” refers to mammalian subjects, including humans, horses, dogs, cats, pigs, livestock, and any other mammal, along with birds. As referred to herein the term “animal” also includes an individual animal in all stages of development, including newborn, embryonic and fetal stages. In embodiments, a present subject is a human.

The term “host” or “organism” as used herein includes humans, mammals (e.g., cats, dogs, horses, etc.), insects, living cells, and other living organisms. A living organism can be as simple as, for example, a single eukaryotic cell or as complex as a mammal. Typical hosts to which embodiments of the present disclosure relate will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For research applications, a wide variety of mammals will be suitable subjects, including rodents (e.g., mice, rats, hamsters), rabbits, primates, and swine such as inbred pigs and the like. Additionally, for in vitro applications, such as in vitro research applications, body fluids and cell samples of the above subjects will be suitable for use, such as mammalian (particularly primate such as human) blood, urine, or tissue samples, or blood, urine, or tissue samples of the animals mentioned for veterinary applications. Hosts that are “predisposed to” condition(s) can be defined as hosts that do not exhibit overt symptoms of one or more of these conditions but that are genetically, physiologically, or otherwise at risk of developing one or more of these conditions.

As used herein, the term “moiety” means a chemical group on a compound or capable of being coupled to a compound that includes a functional group/subunit. As used herein, a “moiety” may include a compound with a specific function that is a part of a larger compound or capable of being coupled to a different compound to form a larger compound.

The terms “protein,” “polypeptide,” and “peptide” may be referred to interchangeably herein. However, the terms may be distinguished as follows. A “protein” typically refers to the end product of transcription, translation, and post-translation modifications in a cell. As used herein a “polypeptide” can refer to a “protein” or a “peptide”. A “peptide”, in contrast to a “protein”, typically is a short polymer of amino acids, of a length typically of 100 or less amino acids. The peptide of the DM does not comprise a known T or B cell epitope and was not designed to be bound by an antibody.

The term “peptide” or “polypeptide” as used herein refers to proteins and fragments thereof. Peptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The peptides of the immunotherapy compounds are not derived from nature, but instead the sequences are designed de novo. The present DM portion of the immunotherapy compound does not comprise peptides which are derivable from the naturally occurring sequences of a protein. A peptide is said to be “derivable from a naturally occurring amino acid sequence” if it can be obtained by fragmenting a naturally occurring sequence, or if it can be synthesized based upon knowledge of the sequence of the naturally occurring amino acid sequence or of the genetic material (DNA or RNA) that encodes this sequence.

The peptides of the immunotherapy compounds do not share substantial homology or identity with naturally occurring proteins or portions thereof (e.g. peptides). The present DM portion of the immunotherapy compound does not comprise peptides with “substantial similarity” with naturally occurring proteins or portions thereof (e.g. peptides). A peptide with substantial similarity includes peptides with at least 70% or greater sequence homology or identity with a peptide having the same number of amino acid residues as the reference peptide.

The compositions, formulations and methods of the present invention may comprise, consist essentially of, or consist of the components and ingredients of the present invention as well as other ingredients described herein. As used herein, “consisting essentially of” means that the compositions, formulations and methods may include additional steps, components or ingredients, but only if the additional steps, components or ingredients do not materially alter the basic and novel characteristics of the claimed compositions, formulations and methods.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

As used herein, “pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

A “pharmaceutical composition” refers to a mixture of one or more of the compounds described herein, derivatives thereof, or pharmaceutically acceptable salts thereof, with other chemical components, such as pharmaceutically acceptable carriers and excipients. One purpose of a pharmaceutical composition is to facilitate administration of a compound to the organism.

As used herein, a “pharmaceutically acceptable carrier” refers to a carrier or diluent that does not cause significant irritation to an organism and does not abrogate the biological activity and properties of the administered compound.

“Physiological condition” refers to conditions of pH and ion concentration found in vivo. “Physiological pH” is generally between 7.2 and 7.5 but p can also as low as pH 6 inside tumors. Physiological ion concentration is generally between 250 to 280 mM.

As used herein, toll-like receptor (TLR) agonists, including TLR7 and/or TLR8, refers to a single stranded RNA or synthetic small molecule that binds or activates TLR. The main target cells of TLR7 agonists are plasmacytoid dendritic cells, producing IFN-α and thus acting on other immune cells. Thereby dendritic cells acquire enhanced costimulatory and antigen-presenting capacity, priming an adaptive immune response. Besides NK cells, antigen-specific T cells are the main terminal effectors of TLR7 agonists in tumor therapy. As used herein a TLR7 and/or TLR8 agonist refers to a synthetic molecule that acts as a ligand for TLR7 and/or TLR8 and includes imidazoquinolines and nucleoside analogs.

As used herein, the term “agonist” indicates a compound that induces a receptor molecule, for instance, a ligand that binds with and activates a receptor molecule. In embodiments of the present disclosure, imidazoquinoline derived compounds of the present disclosure are ligands that can activate certain receptors in a host immune system, such as TLR7 and TLR8, thereby inducing the receptors to generate an immunological response. Thus, in embodiments, the imidazoquinoline derived compounds of the present disclosure can be TLR7 or dual TLR7/TLR8 agonists.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable. In addition, “treat”, “treating”, and “treatment” can also mean prolonging survival as compared to expected survival if not receiving treatment and/or can be therapeutic in terms of a partial or complete cure for a disease and/or adverse effect attributable to the disease. As used herein, the terms “prophylactically treat” or “prophylactically treating” refers completely, substantially, or partially preventing a disease/condition or one or more symptoms thereof in a host. Similarly, “delaying the onset of a condition” can also be included in “prophylactically treating” and refers to the act of increasing the time before the actual onset of a condition in a patient that is predisposed to the condition.

As referred to herein, a “vaccine” can include an antigen or vector, along with other components of a vaccine formulation, including for example adjuvants, slow release compounds, solvents, etc. Although vaccines are traditionally used to prevent or treat infectious diseases, vaccines are also able to modify the function of metabolites by binding signaling peptides or proteins or their receptors and by blocking antigens unique to certain abnormal cell types, such as for example, tumors. Accordingly, it is an embodiment of the invention to provide vaccines to improve immune response to any antigen regardless of the antigen source or its function, including antigens to alter physiological functions that are desirable to improve health, such as immunizing against cancer. In certain embodiments, the present immunotherapy compounds are formulated as a cancer vaccine wherein the immunotherapy compounds induce a cell mediated immune response against antigens present within tumors.

Provided herein are immunotherapy compounds that comprise a solubility component which may be augmented by pH and/or ion concentration of its aqueous environment, and an immunostimulatory (IM) component, such as a TLR7 and/or TLR8 agonist, wherein the two components are connected via a linker, which may be a single covalent bond or a chain, with or without branches, and which may be a cleavable linker.

In embodiments, the solubility component (also referred to herein as the delivery/depot moiety (DM)) is selected to provide the immunotherapy compound with desirable properties under different conditions. For example, it may be desirable that the compound is soluble in a first solution, such as water for injection, histidine buffer solution (for example, 28 mM L-histidine buffer), sodium bicarbonate, Tris-HCl, a phosphate buffer or an acetate buffer in the presence or absence of additional ions in order to allow the compound to be formulated in a pharmaceutical composition. It may then be desirable that the immunotherapy compound has a lower solubility and/or agglomerates in a second solution at higher pH and/or ion concentration, such as a serum, plasma, interstitial fluid or cell culture medium (or a solution that is representative of such a physiological solution, for example: an aqueous sodium chloride solution such as 0.9% sodium chloride solution at close to neutral pH; an aqueous solution of sodium chloride and histidine such as 0.9% sodium chloride in 28 mM L-histidine; or PBS).

In certain embodiments, the solubility component is configured, wherein when conjugated to the IM, to be soluble in an aqueous solution with a pH and/or ion concentration below physiological conditions, e.g. below about pH 7.0 and sodium chloride below about 0.9%, and less soluble, or insoluble, in an aqueous environment with physiological conditions as to higher pH and/or ion concentration. In embodiments, the immunotherapy compounds form self-assemblies or aggregates in an aqueous environment at physiological conditions. Blood has a pH range of about 7.35 to 7.45, while the pH of solid tumors may have a pH range of about 7.0 to 7.4, and the microenvironment around tumors slightly acidic with a pH range of about 6.5 to 6.9. In embodiments, the present immunotherapy compounds are configured to be soluble (in an aqueous solution) at conditions of pH and/or ion concentration below physiological conditions, and insoluble in the form of self-assemblies or aggregates, at physiological conditions.

In some embodiments, the present immunotherapy compounds have the structure of Formula (I): DM-L-IM, wherein DM is the solubility component (and also referred to herein as a delivery/depot moiety, e.g, a peptide such as but not limited to any of SEQ ID NOS. 2-4 and 6-8), L is a linker and IM is the immunostimulatory component. In embodiments, the DM comprises a peptide from about 18 to about 45 amino acids in length comprising amino acid residues possessing helix forming properties wherein the DM is configured to form an amphipathic α-helix structure, and wherein the peptide sequence is not derived from an antigen or immunogen and is a non-natural sequence. In embodiments, the peptide does not comprise a T cell epitope. In other embodiments, the peptide does not comprise a B cell epitope. In certain embodiments, the peptide of the immunotherapy compound does not comprise a T cell epitope or a B cell epitope. Search for T or B cell epitopes can be performed using the immune epitope database at IEDB (www.iedb.org/home_v3.php) containing over 500,000 peptide epitopes at the time of the analysis. The peptide sequences presented in this disclosure were searched and no T or B cell epitopes were identified.

The peptides of the present immunotherapy compounds were designed to provide an alpha-helix structure and that remain soluble in an aqueous solution, when conjugated to the IM, at conditions of pH and/or ion concentration below physiological conditions, but that are less soluble at physiological conditions such that the immunotherapy compounds aggregate or form self-assemblies following administration in vivo. Coupling the DM comprising a peptide to the IM may, for example, reduce the pyrogenicity of the IM compared to the ‘free’ IM, and/or reduce the levels of in vivo inflammatory indicators, such as IL-1, TNF-α, IL-6 and/or IL-8 in the circulation.

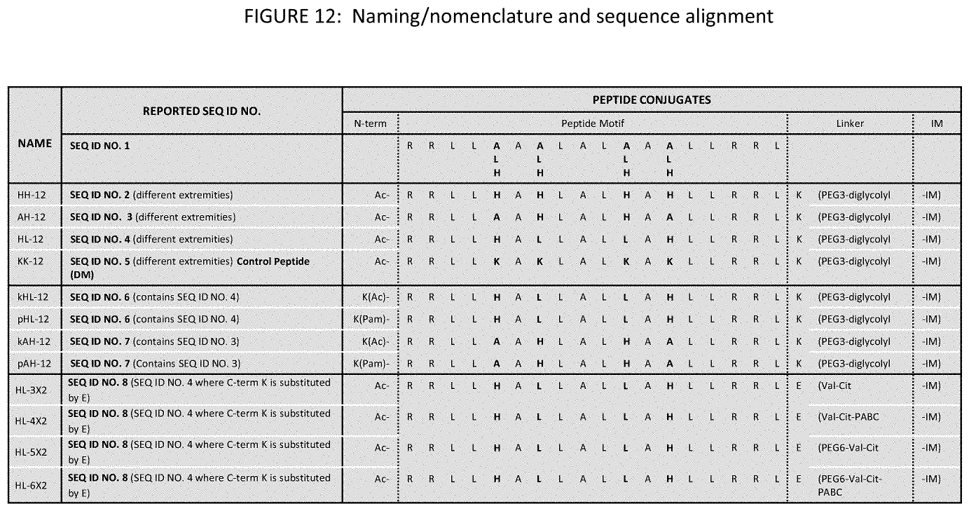

Exemplary DM peptides comprise an amino acid sequence of RRLL(5)A(7)LAL(11)A(13)LLRRL (SEQ ID NO: 1) wherein amino acid positions (5), (7), (11) and (13) are each selected from A, L, or H; in some embodiments, an additional L (leucine) can be included on the C-terminus. See Example 1. Exemplary embodiments of the peptides comprise SEQ ID NO: 2, 3 and 4. Those peptides form an alpha-helix, as depicted in FIGS. 1 to 3 and demonstrated in Example 3 and Table 3, and form aggregates in an environment with physiological conditions as demonstrated in Example 4. See also FIG. 12.

In designing the present DM comprising a peptide, the hydropathic index of amino acids was considered. See Example 1. The importance of the hydropathic amino acid index in conferring interactive biologic function on a peptide is generally understood in the art. It is known that certain amino acids can be substituted for other amino acids having a similar hydropathic index or score and still result in a peptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant peptide, such as an alpha-helix structure. It is known in the art that an amino acid can be substituted by another amino acid having a similar hydropathic index and still obtain a functionally equivalent peptide. In such changes, the substitution of amino acids whose hydropathic indices are within ±2 is preferred, those within ±1 are particularly preferred, and those within ±0.5 are even more particularly preferred.

Amino acid substitutions may be based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Cys: Ser, Ala); (Gln: Asn), (Glu: Asp), (Gly: Ala), (His: Asn, Gin), (Ile: Leu, Val), (Leu: Ile, Val), (Lys: Arg), (Met: Leu, Tyr), (Phe: Leu, Val, Ile, Ala, Tyr); (Pro: Ala); (Ser: Thr), (Thr: Ser), (Trp: Tyr), (Tyr: Trp, Phe), and (Val: Ile, Leu).

It is understood that the SEQ ID NO: 1 can be extended by one or more amino acid residues on its N- or C-terminus provided the peptide construct keeps its ability to form a depot or precipitate under physiological conditions but be soluble at a lower pH and/or ion concentration.

In embodiments, the DM comprising a peptide is covalently linked to the IM, wherein the IM is a toll-like receptor 7 (TLR7) and/or TLR8 agonist. In embodiments, the TLR7 and/or TLR8 agonist is an imidazoquinoline (including a derivative or analog thereof), a thiazoquinoline derivative or analog, a purine-based compound such as adenine or guanine derivatives or analogs, benzazepine derivatives or analogs, pteridinone derivatives or analogs or pyrimidine derivatives or analogs. In embodiments, TLR7 and/or TLR8 agonist contains or is modified by an amino or a carboxyl group to be used for conjugation to another compound. TLR7/TLR8 are innate immune receptors present in the endosomal compartment that are activated by single-stranded RNA (ssRNA) molecules of viral as well as non-viral origin, inducing the production of inflammatory cytokines necessary for the development of adaptive immunity. Molecules that induce TLR7/8 (e.g., agonists) represent potential cancer vaccine targets that can activate a host immune system against cancer antigens present in tumors. Synthetic small molecule agonists of TLR7 and/or TLR8 include the imidazoquinoline class of compounds such as gardiquimod [1-(4-amino-2-((ethylamino)methyl)-1H-imidazo[4,5-c]quinolin-1-yl)-2-methylpropan-2-ol], imiquimod (1-(2-methylpropyl)imidazo[4,5-c]quinolin-4-amine), resiquimod/R848 (1-(4-amino-2-(ethoxymethyl)-1H-imidazo[4,5-c]quinolin-1-yl)-2-methylpropan-2-ol), 4-amino-2-(ethoxymethyl)-1H-imidazo[4,5-c]quinoline-1-butanamine, 1-[[4-(aminomethyl)phenyl]methyl]-2-butyl-1H-Imidazo[4,5-c]quinolin-4-amine.

In some embodiments, present disclosure provides immunotherapy compounds comprising imidazoquinoline-derived compounds chosen from molecules of Formulas (Ia) to (Im) and derivatives and analogues thereof and pharmaceutically acceptable salts thereof, where the immunotherapy compound is capable of activating TLR7 and/or TLR8. In some embodiments, the IM is selected from the molecules represented by Formula (Ia), Formula (Ib), Formula (Ic), Formula (Id), Formula (e), Formula (If), Formula (Ig), Formula (Ih), Formula (Ii), Formula (Ij) Formula (Ik), Formula (Il), and Formula (Im). In embodiments, those formulas have the structure selected from:

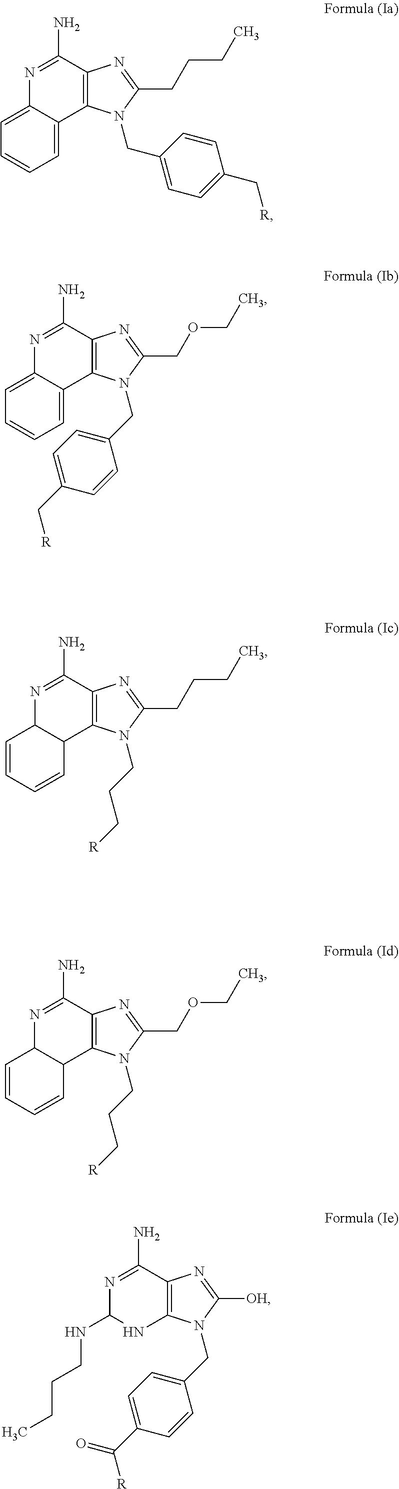

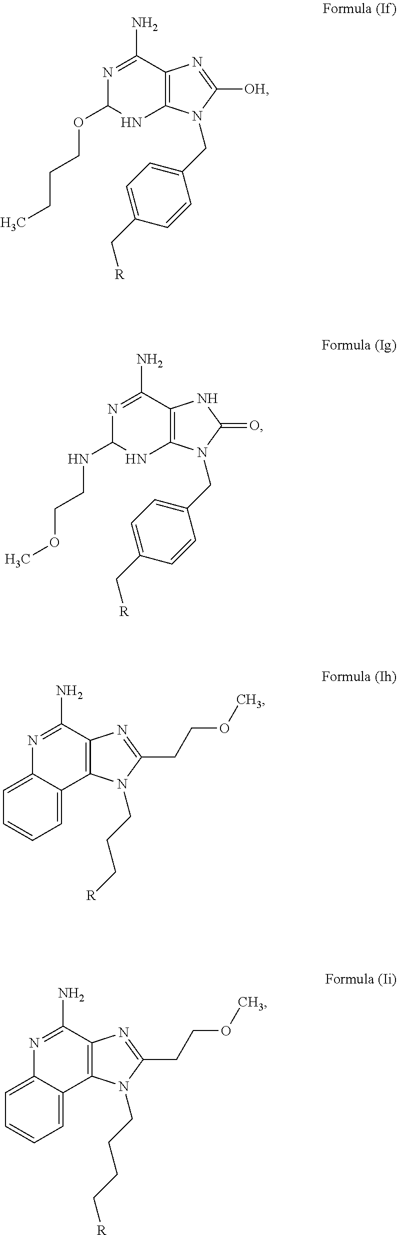

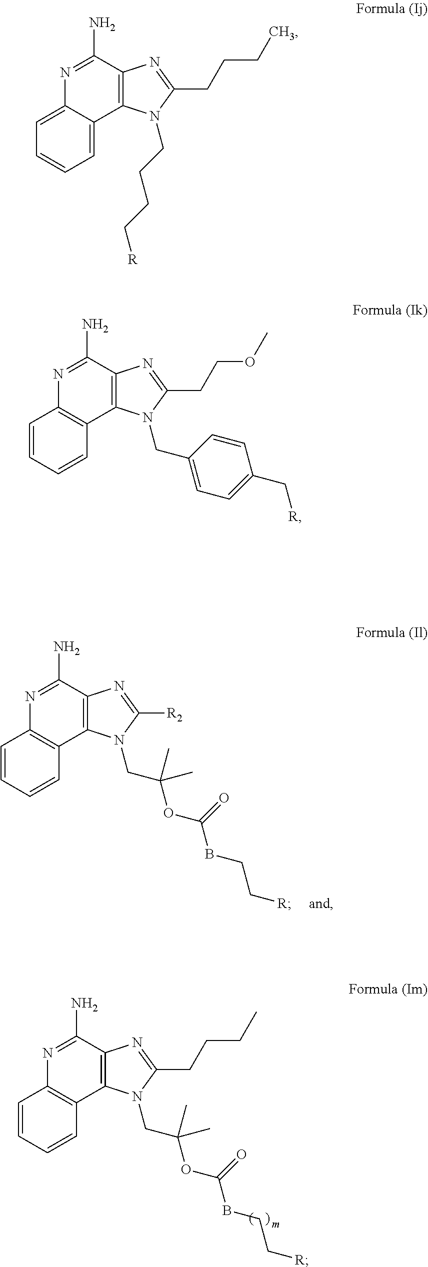

wherein R₂ is selected from:
   —CH₃, —CH₂CH₃, —CH₂CH₂CH₃, —CH₂CH(CH₃)₂, —CH₂CH₂CH₂CH₃ (e.g., as in Formula (Im)) —CH₂CH₂CH₂CH₂CH₃, CH₂OCH₂CH₃, —CH₂CH₂OCH₃, —CH₂NHCH₂CH₃, and —CH₂Ph;
R is the site of conjugation and comprises the linker (L) connecting the IM to an amine group or carboxyl group of the peptide (DM) at a terminal amino acid or the lateral chain of an amino acid such as lysine or glutamine;
B is selected from —O— and —NH—; and,
m is any integer from 1 to 11.

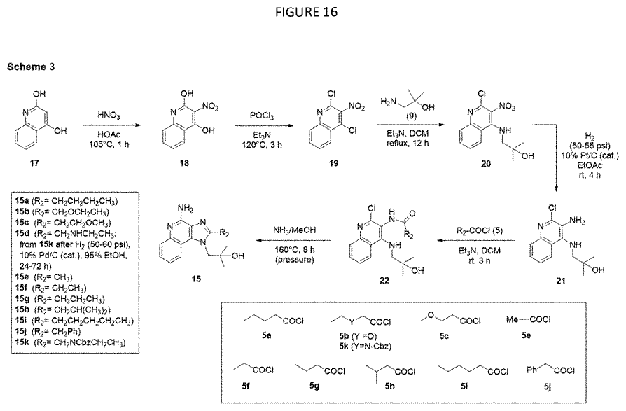

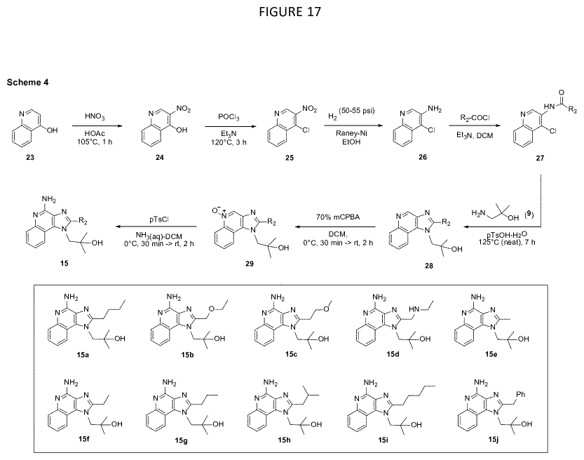

In exemplary embodiments, the present immunotherapy compounds comprise an IM according to any one of Formulas (Ia), (Ib), (Ic), (Id), (Ie), (If), (Ig), (Ih), (Ii), (Ij), (Ik), (Il), (Im). In some embodiments, the present immunotherapy compounds comprise an IM illustrated in FIG. 17. In some embodiments, the present immunotherapy compounds comprise as the IM, and/or be derived from, an imidazoquinoline of Formula 15:

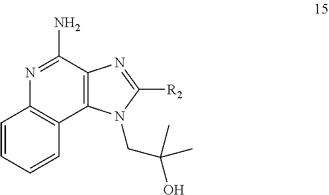

wherein R₂ is CH₂CH₂CH₂CH₃ (15a); R₂═CH₂OCH₂CH₃ (15b); R₂═CH₂CH₂OCH₃ (15c); R₂═CH₂NHCH₂CH₃ (15d); R₂═CH₃ (15e); R₂═CH₂CH₃ (15f); R₂═CH₂CH₂CH₃ (15g); R₂═CH₂CH(CH₃)₂ (15h); R₂═CH₂CH₂CH₂CH₂CH₃ (15i); R₂═CH₂Ph (15j); R₂=CH₂NCbzCH₂CH₃ (15k); and/or, compounds 15a through 15k as illustrated in FIG. 16. In some embodiments, the IM can comprise, and/or be derived from, any one of:

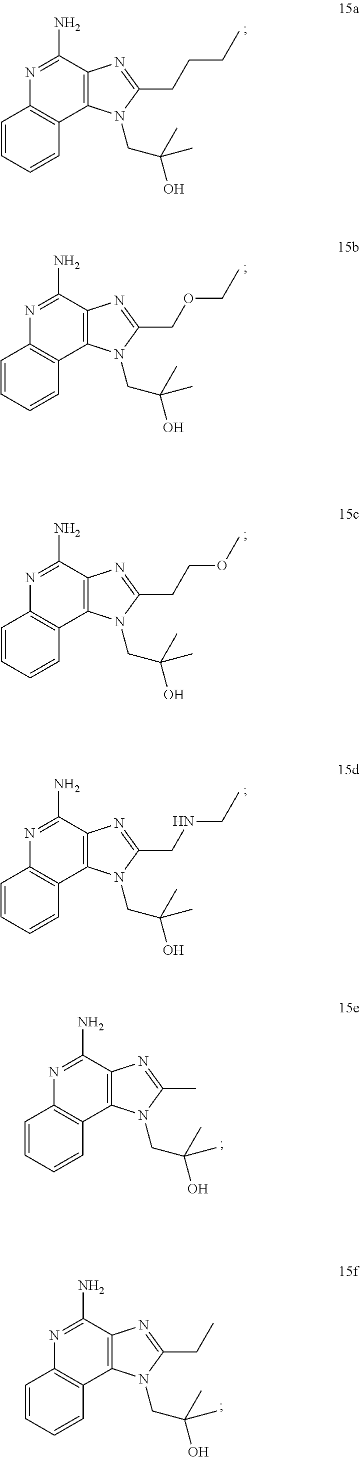 [SMILES: CCCCc1nc2c(N)nc3ccccc3c2n1CC(C)(C)O ; CCOCc1nc2c(N)nc3ccccc3c2n1CC(C)(C)O ; COCCc1nc2c(N)nc3ccccc3c2n1CC(C)(C)O ; CCNCc1nc2c(N)nc3ccccc3c2n1CC(C)(C)O ; Cc1nc2c(N)nc3ccccc3c2n1CC(C)(C)O ; CCc1nc2c(N)nc3ccccc3c2n1CC(C)(C)O]

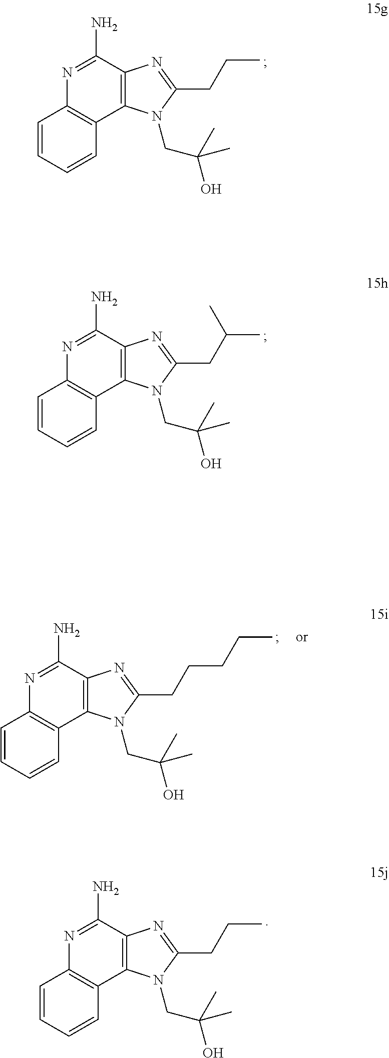 [SMILES: CCCc1nc2c(N)nc3ccccc3c2n1CC(C)(C)O ; CC(C)Cc1nc2c(N)nc3ccccc3c2n1CC(C)(C)O ; CCCCCc1nc2c(N)nc3ccccc3c2n1CC(C)(C)O ; CCCc1nc2c(N)nc3ccccc3c2n1CC(C)(C)O]

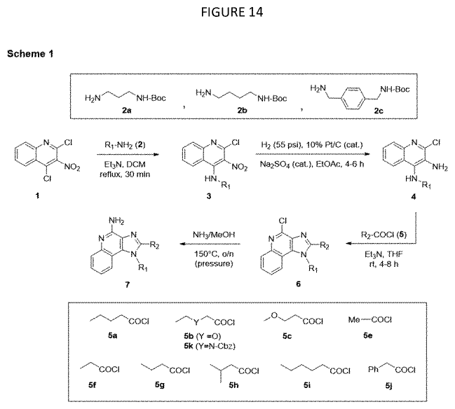

In some embodiments, the IM can be synthesized by or using any of Schemes 1-6 as illustrated in FIGS. 14-19, and/or as described in Examples (e.g., using one or more of Methods A through F). For instance, in some embodiments, as shown in Example 7, Method A can be used for IM in formulas (Ia), (Ib), (Ic), (Id), (Ih), (Ii), (Ij) and (Ik), while Methods B, C, D or E in conjunction with Method F can be employed to synthesize an IM of formulas (Il) and (Im). Other methods may also be suitable as would be understood by those of ordinary skill in the art.

In some embodiments, as illustrated in Scheme 1 (FIG. 14, Method A of Example 7), a solution of 2,4-dichloro-3-nitroquinoline (1, 1.0 eq) in anhydrous dichloromethane (DCM), triethylamine (Et₃N) and the mono-protected diamine (2) (preferably 2a, 2b, or 2c (FIG. 14)) can be used to produce purified compound 3; after which catalytic amounts of 10% platinum on carbon (10% Pt/C) and sodium sulfate (Na₂SO₄) can be used to obtain crude compound 4 from compound 3; after which Et₃N and acid chloride (compound 5 (preferably 5a, 5b, 5c, 5d, 5e, 5f, 5g, 5h, 5i, 5j or 5k (FIG. 14)) can be used to produce crude compound 6 from compound 4; after which methanol (MeOH), ammonia (NH₃), pressure and heat can be used to produce compound 7 from compound 6. In some embodiments, combinations of compounds 2 and 5 can be used to produce IMs of Formulas (Ia), (Ib), (Ic), (Id), (Ih), (Ii), (Ij) and (Ik), respectively, as shown in Table 5 of Example 7. Variants of this method may also be suitable as may be determined by those of ordinary skill in the art using routine techniques.

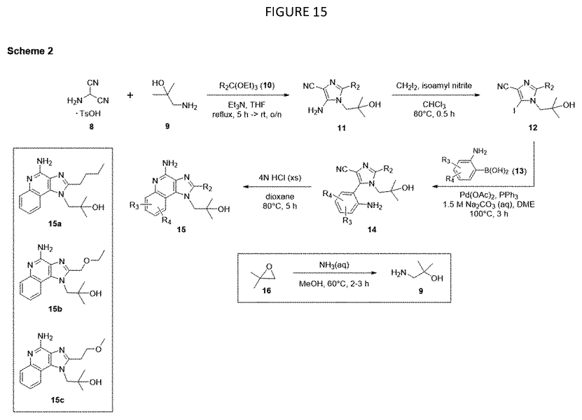

In some embodiments, as illustrated in Scheme 2 (FIG. 15, Method B of Example 7), aminomalononitrile p-toluenesulfonate (8) can be treated with Et₃N, followed by the addition of orthoformate (10), and heated, after which additional orthoformate (10) can be added followed by additional heating. After cooling, Et₃N and then 1-amino-2-methylpropan-2-ol (9) (produced, e.g., as shown in FIG. 15) can be added, stirred, concentrated, dissolved, washed, saturated with Na₂CO₃, extracted (e.g., with DCM), saturated (e.g., with aqueous brine (NaCl)), dried, filtered, evaporated, and purified to produce compound 11. Compound 11 can then be heated after which isoamylnitrite (4.0 eq) in chloroform (CHCl₃) can be added and processed (e.g., heating, cooling, concentrating, purifying) to produce compound 12. Catalyst (e.g., palladium acetate (Pd(OAc)₂) can then be introduced into a suspension with compound 12. Compound 13 and Na₂CO₃ are added, followed by processing (e.g., heating, cooling, diluting, extracting, washing, drying, filtering, concentrating and purifying) to produce compound 14. Compound 14 can then be mixed with HCl in dioxane, heated, cooled, and concentrated, followed by taking the residue up in MeOH (e.g., 10%) in EtOAc. The combined organic layers can then be processed by washing, drying, filtering, evaporation, and purifying the resultant crude residue to produce compound 15 (e.g., 15a, 15b, or 15c (FIG. 15)). In one embodiment, compound 15a can be synthesized from 1,1,1-triethoxypentane (triethyl orthovalerate, 10a, R₂═CH₂CH₂CH₂CH₃) and 2-aminophenylboronic acid (13a, R₃, R4═H). Analogous compounds can be made with different R₂, R₃ and R₄ groups on 10 and 13, respectively, using this Method B as well. For instance, in some embodiments, a number of substituted 2-aminophenylboronic acids (13) in which R₃ is H, Me, Et, iPr, tBu, cyclopropyl, CF₃, F, Cl, Br, NO₂, OPG₁, OMe, OCF₃, NHPG₁, NMePG₂, NEtPG₂, NHCOMe, CN, CO₂PG₃, CO₂Me, CO₂Et, CO₂iPr, CONHMe, CONHEt, or SO₂Me; and R₄ is H, Me, CF₃, F, Cl, NO₂, OPG₂, OMe, OCF₃, CN, CO₂PG₃; where PG₁ is H, tBu, CH₂Ph, TBDMS, COMe; PG₂=H, Alloc, Boc, Cbz, Fmoc; and PG₃ is H, tBu, or CH₂Ph can be used and are available commercially. Ortho esters (10) can be used where, for example, R₂ is CH₃, CH₂CH₃, CH₂CH₂CH₃, CH₂CH₂CH₂CH₃, CH₂OCH₂CH₃, CH₂CH₂OCH₃, and can be made relatively easily using the Pinner reaction (McElvain, S. M.; Nelson, W. J. Am. Chem. Soc. 1942, 64, 1825-1827; Roger, R.; Neilson, D. G. Chem. Rev. 1961, 61, 179-211; Noe, M.; Perosa, A.; Selva, M. Green Chem. 2013, 15, 2252-2260) from nitriles and alcohols under acidic conditions or in the presence of a Lewis acid, such as BF₃-etherate (Corey, E. J.; Raju, N. Tetrahedron Lett. 1983, 24, 5571-5574), with a limited selection also obtainable from organic chemical reagent vendors. Aryl boronic acids (13) can also be prepared from organometallic reagents (i.e. Grignards, organolithiums) and trialkyl borates (“Synthesis of Organoboronic Acids, Organoboronates, and Related Compounds” Chapter 6, in Practical Functional Group Synthesis, Stockland, R. A., Jr., John Wiley & Sons, Inc., Hoboken, N.J., 2016, pp 515-555). Lastly, imidazoquinolines with different N1-substituents can also be made through the process outlined in Method B through the use of different amino alcohols or diamines (Lason, P.; Kucaba, T. A.; Xiong, Z.; Olin, M.; Griffith, T. S.; Ferguson, D. M. ACS Med. Chem. Lett. 2017, 8, 1148-1152), such as 2a, 2b and 2c. Hence, Method B also has applicability to the IMs of Formulas (Ia), (Ib), (Ic), (Id), (Ih), (Ii), (Ij) and (Ik). Variants of such methods may also be suitable as may be determined by those of ordinary skill in the art using routine techniques.

In some embodiments, as illustrated in Scheme 3 (FIG. 16, Method C of Example 7), 2,4-dihydroxyquinoline (17), nitric acid (HNO₃), glacial acetic acid (HOAc), and water is used to produce compound 18; which is then processed using phosphorous oxychloride (POCl₃), and Et₃N to produce compound 19. Compound 20 can then be produced from compound 19 using Et₃N and 1-amino-2-methylpropan-2-ol (9); which is then processed using EtOAc, hydrogen gas, and a platinum catalyst to produce compound 21. Compound 21 can then be reacted with Et₃N and the acid chloride (5) (preferably one of acid chlorides 5a, 5b, 5c, 5e, 5f, 5g, 5h, 5i and 5j (FIG. 16)) to produce compound 22; which is then mixed with NH₃-MeOH and processed (e.g., using pressure) to produce compound 15 (e.g., imidazoquinoline 15a, 15b, 15c, 15e, 15f, 15g, 15h, 15i and 15j using acid chlorides 5a, 5b, 5c, 5e, 5f, 5g, 5h, 5i and 5j, respectively (FIG. 16)). In addition, use of acid chloride 5k provides imidazoquinoline 15k possessing an N-Cbz protecting group, which can be removed employing the procedure described in Step E-12 (Example 7) to yield 15d. Variants of these methods may also be suitable as may be determined by those of ordinary skill in the art using routine techniques.

In some embodiments, as illustrated in Scheme 4 (FIG. 17, Method D of Example 7), synthesis of 15 begins with the nitration of 4-hydroxyquinoline (23) using the method of Step C-1 (Example 7); the resulting 3-nitro-4-hydroxy quinoline (24) is then subjected to a sequence involving chlorination (Step C-2 (Example 7)) to provide compound 25; followed by reduction of the nitro group with hydrogen gas, Raney nickel in ethanol (e.g., Step C-4 (Example 7)) to give compound 26, and finally acylation (Step C-5 (Example 7) to yield compound 27; high temperature and concentrated reaction conditions are then used to produce an acid-induced, dehydrative ring closure of compound 27 experienced essentially simultaneously with displacement of the chloride with the amine of 1-amino-2-methylpropan-2-ol (9) to produce compound 28; to which is then added meta-chloroperoxy-benzoic acid and further processed to produce product 29; which is then treated with concentrated ammonium hydroxide (NH₄OH, NH₃(aq))), followed by p-toluenesulfonyl chloride (Tos-Cl) to produce product 15. This process can be employed with acid chlorides 5a, 5b, 5c, 5e, 5f, 5g, 5h, 5i and 5j to give the imidazoquinolines 15a, 15b, 15c, 15e, 15f, 15g, 15h, 15i and 15j, respectively (FIG. 17). Similar to Method C (Example 7), the procedure of Step E-12 (Example 7) can be employed to convert 15k (made from 5k) into 15d. Variants of these methods may also be suitable as may be determined by those of ordinary skill in the art using routine techniques.

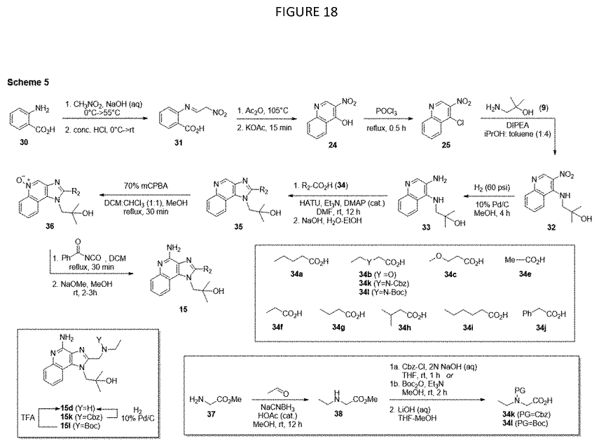

In some embodiments, as illustrated in Scheme 5 (FIG. 18, Method E of Example 7), 2-nitroacetaldehyde oxime is prepared, acidified with HCl (conc.), added to anthranilic acid (30), and processed to produce product 31; which is then processed using acetic anhydride then potassium acetate to obtain 3-nitroquinolin-4-ol (24); compound 24 is then processed using phosphorous oxychloride, heated, evaporated, filtered, washed, dried and the residue triturated with diethyl ether to give 4-chloro-3-nitroquinoline (25); compound 25 is then treated with 1-amino-2-methylpropan-2-ol (9), N,N-diisopropylethylamine (DIPEA) in toluene and isopropanol (iPrOH) with heat to produce a precipitate which is cooled, filtered and washed sequentially with toluene:iPrOH (7:3), diethyl ether and cold H₂O, and dried to obtain compound 32; compound 32 then is dissolved in MeOH and hydrogenated using 10% Pd/C as catalyst under a H₂ pressure, filtered and evaporated in vacuo to leave compound 33; which is followed by dissolving 33, 34 (e.g. 34a, 34b, 34c, 34d, 34e, 34f, 34g, 34h, 34i, or 34j), O-(7-azabenzotriazol-1-yl)-N,N,N′,N′-tetramethyl uronium hexafluorophosphate (HATU) (1.4 eq), Et₃N (3.5 eq) and 4-dimethylaminopyridine (DMAP) (cat.) in dimethylformamide (DMF), stirred, evaporated, the residue dissolved in EtOAc, washed, dried and filtered and heated to produce compound 35. In some embodiments, to a solution of 35 in DCM:CHCl₃ (1:1) and MeOH (10% by volume) is added meta-chloroperoxybenzoic acid and the reaction heated to reflux for 30 min, followed by concentration and purification to give the N-oxide 36. In some embodiments, 36 is dissolved in anhydrous DCM and benzoyl isocyanate added, and then the mixture is heated, followed by concentration in vacuo and dissolution in anhydrous MeOH; excess NaOMe is then added, the reaction is refluxed for 2-3 h, and, following evaporation, the crude residue is purified using flash chromatography to obtain 15. These processes can be employed with acids 34a, 34b, 34c, 34e, 34f, 34g, 34h, 34i and 34j to give the imidazoquinolines 15a, 15b, 15c, 15e, 15f, 15g, 15h, 15i and 15j, respectively. Imidazoquinolines 15k and 15l, accessed from acids 34k and 34l, respectively, are deprotected as described using the procedures in Steps E-11 (Example 7) and E-12 (Example 7), respectively, to yield 15d (FIG. 18). Variants of these methods may also be suitable as may be determined by those of ordinary skill in the art using routine techniques.

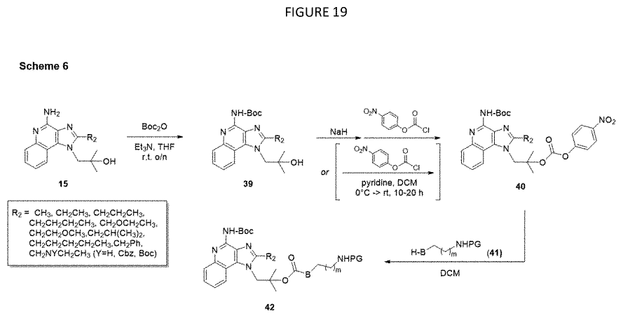

In some embodiments, the structure required for formula (Il) can be accessed from 15 using the synthetic route shown in Scheme 6 (FIG. 19, Method F of Example 7), in which the 4-amino group of 15 (R₂═CH₃, CH₂CH₃, CH₂CH₂CH₃, CH₂CH₂CH₂CH₃, CH₂OCH₂CH₃, CH₂CH₂OCH₃,CH₂CH(CH₃)₂, CH₂CH₂CH₂CH₂CH₃, CH₂Ph, CH₂NYCH₂CH₃ (Y=H, Cbz, Boc)) is first protected so as to not interfere in subsequent chemistry using a standard method for its installation (e.g. Boc₂O, Et₃N, as in Step E-11 (Example 7)), to produce the Boc-protected product 39, which is then activated as its p-nitrophenyl carbonate by treatment with p-nitrophenyl chloroformate in the presence of base, with two alternative reactions for this transformation shown in FIG. 19. Other activated moieties can also be used here, such as pentafluorophenyl (OPfp) or succinimide (OSu). Compound 40 can then be reacted with N-mono-protected diamines (41a, B=—NH—) or amino alcohols (41b, B=—O—) to produce the urethanes (42a, B=—NH—) or carbonates (42b, B=—O—), respectively. The protecting group (PG) on 41 is preferably orthogonal to the Boc (i.e. Cbz, Fmoc, Alloc), but could also even be Boc, since at this stage, the 4-amino group does not require protection as its free state is not expected to interfere with subsequent transformations, so its removal should not be detrimental. However, the N-protection (Y=Cbz, Boc) on the C2-substituent of 15, when present, must remain in place so that this secondary amine does not interfere with the chemistry utilized in the formation of the conjugates. Deprotection of PG in 42 then provides the structure required for Formula (Il). Variants of these methods may also be suitable as may be determined by those of ordinary skill in the art using routine techniques.

In some embodiments, the linker is -[A1]—NH— and A1 is selected from:
A2-A3-(CH₂)ₓ—CO—,
A2-A3-CH₂—O—CH₂—CO—,
A2-A3-(CH₂)ₓ—O—(CH₂)ₓ—O—(CH₂)ₓ—O—(CH₂)ₓ—CO—,
A2-Valine-Alanine-A4-,
A2-Valine-Citrulline-A4-,
A2-Glutamate-Valine-Citrulline-A4-, or
A2-Phenylalanine-Lysine-A4-; wherein:
A2 is selected from:
A5-(CH₂)ₓ-A6-, -A5-(CH₂)ₓ—O—(CH₂)ₓ—O—(CH₂)ₓ—O—(CH₂)ₓ-A3-,
A5-(CH₂)₂-(O—CH₂—CH₂)ₓ-A6-
A5-CH₂—O—CH₂-A6-,
A5-(CH₂)ₓ-A6-,
A5-(CH₂)ₓ—O—(CH₂)ₓ—O—(CH₂)ₓ—O—(CH₂)ₓ-A6-, or,
A5-NH—(CH₂)₂—O—(CH₂)-A6-;
A3 is —CO— or —NH—;
A4 is p-aminobenzyloxy carbonyl (PABC),

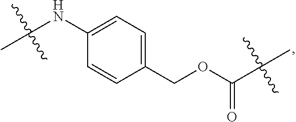 [SMILES: *Nc1ccc(COC(*)=O)cc1]

or nothing;
A5 and A6 are —CO— or —NH—, one or more natural or non-natural amino-acids, or nothing; and,
x is any integer from 1 to 12, preferably 2 to 12.

In exemplary embodiments the present immunotherapy compounds are represented by the peptide conjugates of Tables 2A, 2B and 2C (Example 2).

In embodiments, the present immunotherapy compounds comprise a hydrophobic moiety wherein the DM further comprises a hydrophobic moiety covalently attached to a terminal amino acid of the peptide.

In certain embodiments, the hydrophobic moiety is a hydrocarbon including, but not limited to, fatty acids such as palmitoyl, myristoyl, stearoyl and decanoyl groups or, more generally, any saturated, monounsaturated or polyunsaturated fatty acyl group.

In certain embodiments, the hydrophobic moiety is a hydrocarbon chain substituted with one or more halogen atoms. In embodiments, the hydrophobic moiety is a hydrocarbon chain substituted with one or more fluorine atoms, herein referred to as a “fluorocarbon chain”.

The fluorocarbon can comprise one or more chains derived from perfluorocarbon or mixed fluorocarbon/hydrocarbon radicals, and may be saturated or unsaturated, each chain having from 3 to 30 carbon atoms. Thus, the chains in the fluorocarbon attachment are typically saturated or unsaturated, preferably saturated. The chains in the fluorocarbon attachment may be linear or branched, but preferably are linear. Each chain typically has from 3 to 30 carbon atoms, from 5 to 25 carbon atoms, or from 8 to 20 carbon atoms. In order to covalently link the fluorocarbon vector to the peptide, a reactive group, or ligand, for example —CO—, —NH—, S, O or any other suitable group is included in the hydrophobic moiety. The use of such ligands for achieving covalent linkages is well known in the art. The reactive group may be located at any position on the fluorocarbon chain.

Coupling of the fluorocarbon or hydrocarbon chain to the peptide may be achieved through functional groups such as —OH, —SH, —COOH and —NH₂, naturally present or introduced onto any site of the peptide. Examples of such linkages include amide, hydrazone, disulphide, thioether and oxime bonds.

Optionally, a spacer element (peptidic or non-peptidic) can be incorporated to tune its stability and/or solubility. Examples of spacers include a linear or non-linear chain comprising one or more carbon, polyethylene glycol (PEG) or amino acids that may be cleaved by proteolytic enzymes.

In certain embodiments, the fluorocarbon-linked peptide can have the chemical structure CₘFₙ—C_{y}Hₓ (Sp)-R or derivatives thereof, where m=3 to 30, n≤2m+1, y=0 to 15, x≤2y, (m+y)=3 to 30 and Sp is an optional chemical spacer moiety and R is an immunogenic peptide. Typically, m and n satisfy the relationship 2m−1≤n≤2m+1, and preferably n=2m+1. Typically, x and y satisfy the relationship 2y−2≤x≤2y, and preferably x=2y. Preferably the CₘFₙ—C_{y}Hₓ moiety is linear.

In embodiments, m is from 5 to 15, more preferably from 8 to 12. In other embodiments, y is from 0 to 8, more preferably from 0 to 6 or 0 to 4. In embodiments, the CₘFₙ—C_{y}Hₓ moiety is saturated (i.e., n=2m+1 and x=2y) and linear, and that m=8 to 12 and y=0 to 6 or 0 to 4.

In certain embodiments, the fluorocarbon chain is derived from 2H, 2H, 3H, 3H-perfluoroundecanoic acid of the following formula:

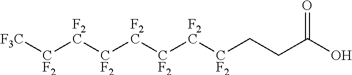 [SMILES: O=C(O)CCC(F)(F)C(F)(F)C(F)(F)C(F)(F)C(F)(F)C(F)(F)C(F)(F)C(F)(F)F]

In embodiments, the fluorocarbon attachment is the linear saturated moiety C₈F₁₇(CH₂)₂ which is derived from C₈F₇(CH₂)₂COOH. In certain embodiments, the fluorocarbon attachments have the following formulae: C₆F₁₃(CH₂)₂—, C₇F₁₅(CH₂)₂, C₉F₁₉(CH₂)₂—, C₁₀F₂₁(CH₂)₂, C₅F₁₁(CH₂)₃—, C₆F₁₃(CH₂)₃—, C₇F₁₅(CH₂)₃—, C₈F₁₇(CH₂)₃ and C₉F₁₉(CH₂)₃ which are derived from C₆F₁₃(CH₂)₂COOH, C₇F₁₅(CH₂)₂COOH, C₉F₁₉(CH₂)₂COOH, C₁₀F₂₁(CH₂)₂COOH, C₅F₁₁(CH₂)₃COOH, C₆F₁₃(CH₂)₃COOH, C₇F₁₅(CH₂)₃COOH, C₈F₁₇(CH₂)₃COOH and C₉F₁₉(CH₂)₃COOH, respectively.

In certain embodiments, the fluorocarbon or hydrocarbon attachment may be modified such that the resulting compound is still soluble at non-physiological conditions and insoluble (e.g. forms self-assemblies and/or aggregates) in a physiological environment. Thus, for example, a number of the fluorine atoms may be replaced with other halogen atoms such as chlorine, bromine or iodine. In addition, it is possible to replace a number of the fluorine atoms with methyl groups or hydrogen and still retain the properties of the molecule described herein.

In embodiments, the peptides may be linked to the fluorocarbon or hydrocarbon chain via a spacer moiety. In one embodiment, the spacer moiety is a lysine residue. This spacer residue may be present in addition to any terminal lysine residues as described above, so that the peptide may, for example, have a total of four N-terminal lysine residues. Accordingly, in certain embodiments, the immunotherapy compounds of the invention may comprise fluorocarbon-linked peptides in which the peptides have a C-terminal or N-terminal lysine residue, preferably an N-terminal lysine residue. In embodiments, the terminal lysine in the peptides is linked to a fluorocarbon having the formula CF₁₇ (CH₂)₂COOH. In embodiments, the fluorocarbon is coupled to the epsilon chain of the N-terminal lysine residue.

In certain embodiments, the hydrophobic moiety is selected from CF₁₇—(CH₂)₂—CO—, CH₃(CH₂)₁₂CO—, CH₃(CH₂)₁₄CO—, CH₃(CH₂)₁₆CO—, or

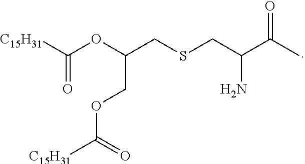

In some embodiments, the immunotherapy compound(s) can be and/or include a peptide conjugate as exemplified in the Examples section. In preferred embodiments, the immunotherapy compounds can be and/or include any one or more of:

Ac-RRLLHAHLALHAHLLRRLK(ADJ12)-NH₂(named HH-12),

Ac-RRLLAAHLALHAALLRRLK(ADJ12)-NH₂(named AH-12),

Ac-RRLLHALLALLAHLLRRLK(ADJ12)-NH₂(named HL-12),

K(Ac)—RRLLHALLALLAHLLRRLK(ADJ12)-NH₂(named kHL-12),

K(Ac)—RRLLAAHLALHAALLRRLK(ADJ12)-NH₂(named kAH-12),

K(Pam)-RRLLHALLALLAHLLRRLK(ADJ12)-NH₂(named pHL-12), or

K(Pam)-RRLLAAHLALHAALLRRLK(ADJ12)-NH₂(named pAH-12),

where Pam=Palmitoyl, Ac=Acetyl

and ADJ12 is:

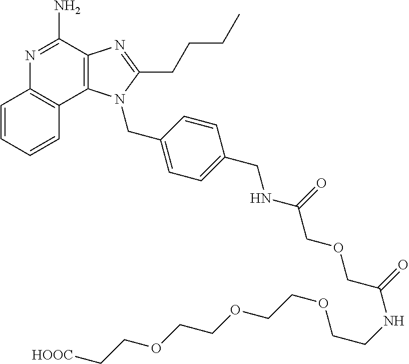 [SMILES: CCCCc1nc2c(N)nc3ccccc3c2n1Cc1ccc(CNC(=O)COCC(=O)NCCOCCOCCOCCC(=O)O)cc1]

where ADJ12 is derived from Formula I(a) where R is —NH—CO—CH₂—O—CH₂—CO—NH—((CH₂)₂O)₃—(CH₂)₂—COOH.

In some embodiments, the immunotherapy compound is selected from the group consisting of:

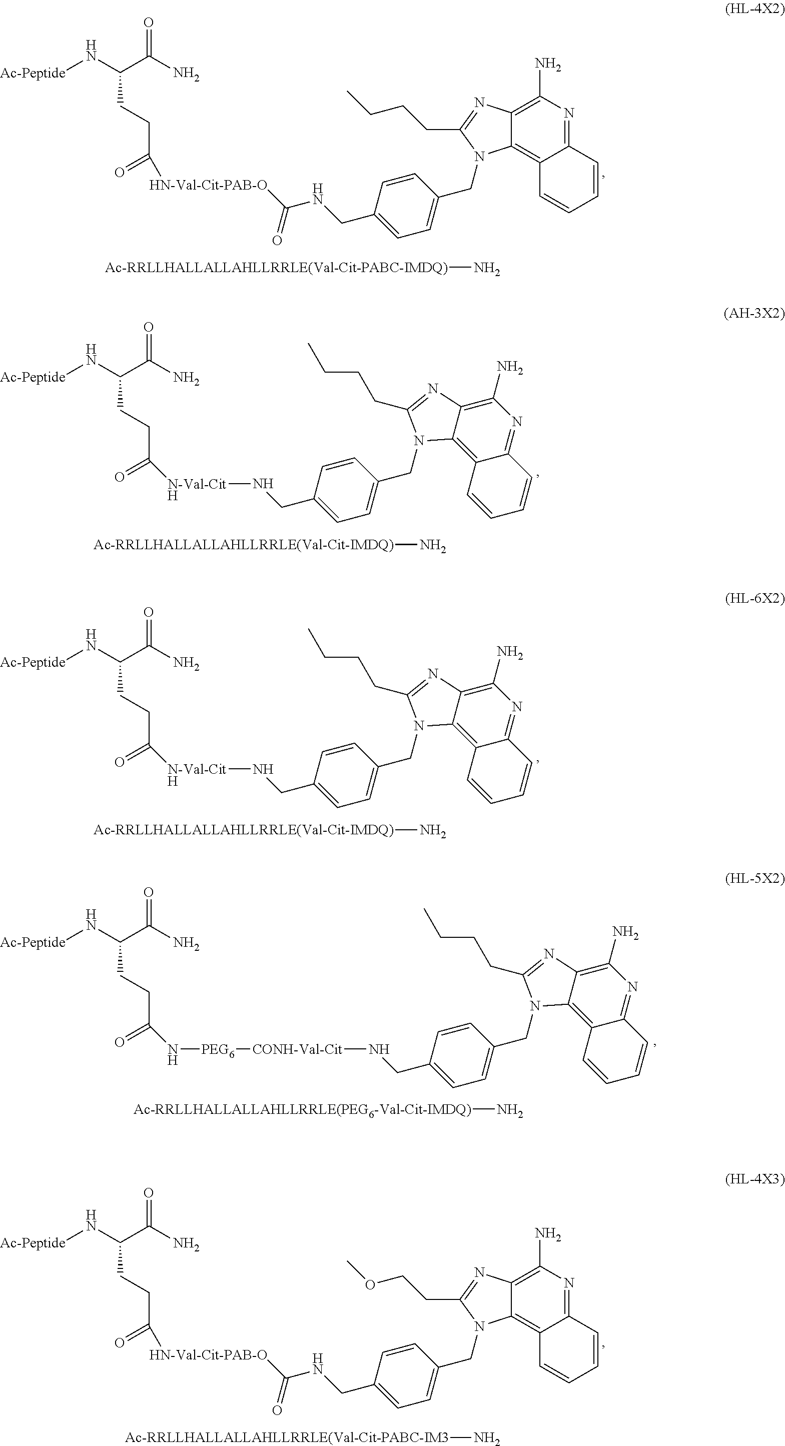

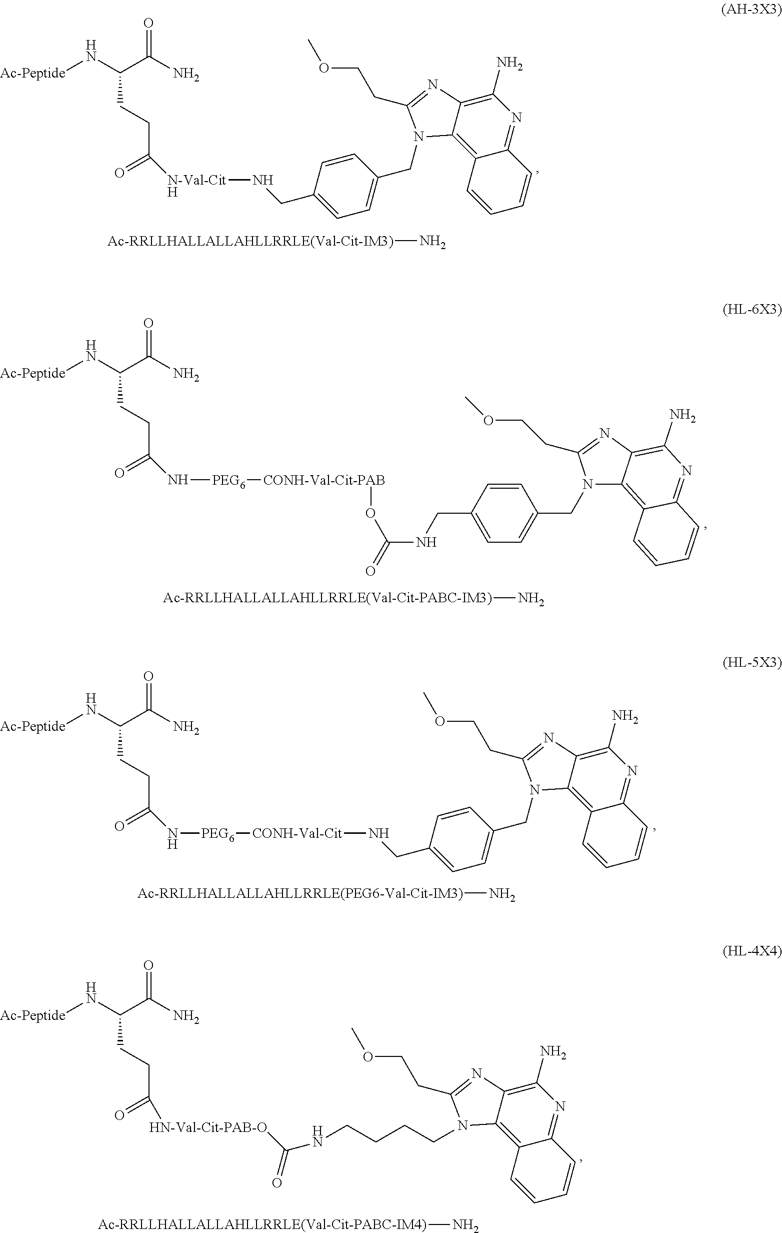

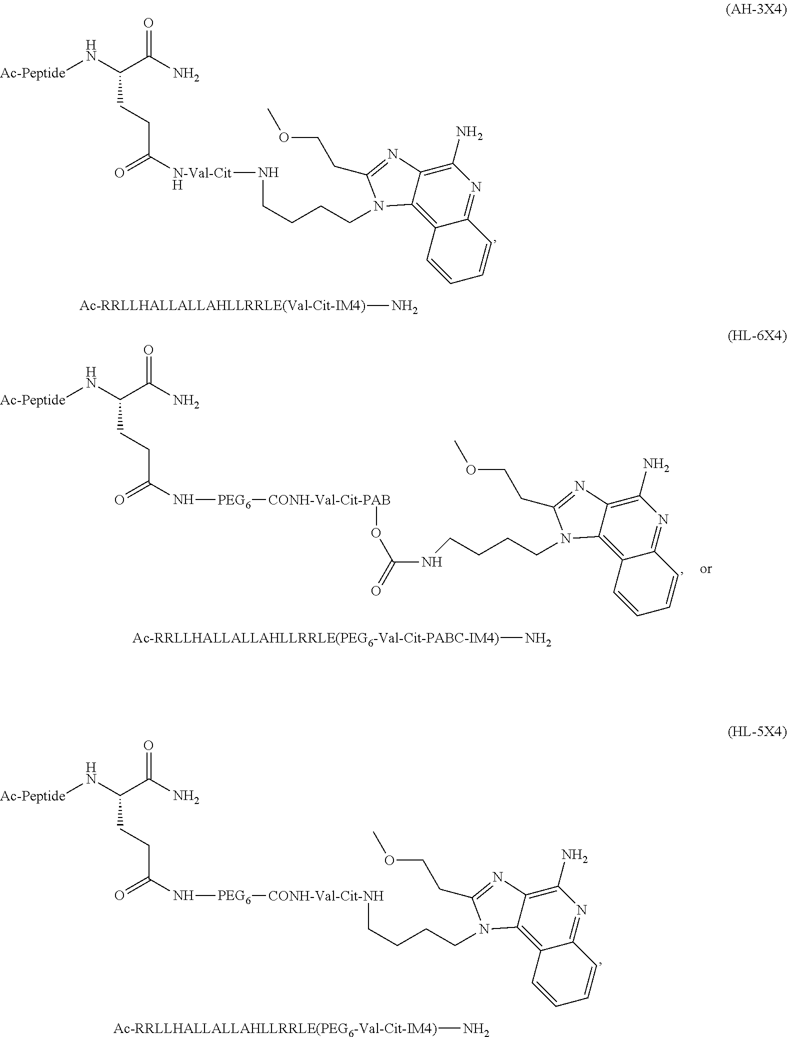

where Ac is Acetyl, Val is valine, Cit is Citrulline, PEG₆ is —NH—(CH₂)₂—(O—CH₂—CH₂)₆—CO, PABC is p-aminobenzyloxy carbonyl, PAB is p-aminobenzyloxy, and IMDQ is Formula (Ia) were R is —NH—; IM3 is Formula (Ik) where R is —NH—; IM4 is Formula (Ii) where R is —NH—; and/or, the immunotherapy compound is as illustrated in FIGS. 12 and/or 13. In preferred embodiments, the immunotherapy compound is selected from the group consisting of kHL-12, HL-6X2, and HL-6X3.

In embodiments, the immune stimulant is a dual TLR7 and TLR8 agonist equivalent in activity to the imidazoquinoline moiety of Formula I(a). Other embodiments of immunotherapy compounds, including and/or derived from those shown herein (e.g., in some embodiments comprising a hydrophobic lipid tail such as a hydrocarbon or fluorocarbon moiety) are also contemplated as would be understood by those of ordinary skill in the art.

Provided herein are immunotherapy compounds formulated as a pharmaceutical composition comprising the present compounds and a pharmaceutical acceptable carrier or diluent. In embodiments, the immunotherapy compound is soluble in an aqueous solution having a pH range of aqueous solution having a pH range of about 3 to 9 (in embodiments, about 4 to 8) or an ion concentration ranging from 0 mM to 600 mM (in some embodiments to about 400 mM). In certain embodiments, the immunotherapy compound is soluble in an aqueous solution having a pH range of about 3 to 9 (in some embodiments, about 4 to 7) independent of the ion concentration. In certain embodiments, the immunotherapy compound is soluble in an aqueous solution having an ion concentration ranging from 0 mM to 400 mM, independent of the pH of the solution. In embodiments, the present immunotherapy compounds are soluble or insoluble based on the pH and ion concentration range of the graph below.

In embodiments, the diluent may comprise a stabilizer or bulking agent necessary for efficient lyophilization. Examples include sorbitol, mannitol, polyvinylpyrrolidone, trehalose, lactose, sucrose, glucose, polyethylene glycol and mixtures thereof, preferably mannitol. Other excipients that may be present include preservatives such as antioxidants, lubricants, cryopreservatives and binders well known in the art.

The pharmaceutical compositions of the invention can be prepared in any standard manner known in the art. For example, the components of the pharmaceutical composition may be solubilized to disperse the components and form a clear, homogeneous solution. This solution may be sterilized, such as by filtration, and then dried.

The term “solubilization” is used herein to mean the dispersion of the compound, and optionally other components of the composition, in a solvent to form a visually clear solution that does not lose material upon sterile filtration. By “dispersion” is meant dissolution of the compound, and optionally other components of the composition, in order to disrupt particulates and achieve solubility.

The input components for the pharmaceutical composition may be blended homogenously together to the desired ratios with any aggregates dispersed, rendered sterile and presented in a suitable format for administration. Such examples could include the introduction of a vortexing and/or sonication post-blending or post-dilution stage to facilitate solubilization. Other permutations of the manufacturing process flow could include sterile filtration being performed at an earlier stage of the process or the omission of lyophilization to permit a liquid final presentation.

Examples of solvents that may be used to disperse the compound in the blend include phosphate buffered saline (PBS), propan-2-ol, tert-butanol, acetone, acetic acid and other organic solvents.

Where more than one solvent is used in the manufacturing process, each solvent used is typically able to solubilize the component it is being used to solubilize at relatively high concentrations (for example, up to 10 millimolar, such as up to 2 millimolar); water-miscible to facilitate dilution with water prior to lyophilization; compatible with lyophilization stabilizers, such as mannitol, that may be used in the manufacturing process; has a safety profile acceptable to the pharmaceutical regulatory authorities, for example, complies with the requirements of ICH Q3C (Note for Guidance on Impurities: Residual Solvents) and the requirements of Class III solvents, as defined by USP Residual Solvents <467> (residual solvent limit of 50 mg/day in finished product or less than 5000 ppm or 0.5%); amenable to lyophilization, that is, sufficiently volatile to be removed to safe levels upon lyophilization; able to disperse the component molecules efficiently in a reproducible and uniform manner such that yield losses on sterilizing grade filtration are minimized; unable to react with, or promote degradation of, the compound or component; and/or compatible with the materials routinely used in pharmaceutical product manufacture (containers/filter membranes/pipework, etc.).

After solubilization and blending, the solution of the compound and optionally other components may be diluted. For example, the blend may be diluted in water.

The solution containing the compound is preferably sterilized. Sterilization is particularly preferred where the formulation is intended for in vivo use. Any suitable means of sterilization may be used, such as heat sterilization, UV sterilization irradiation or filter sterilization. Preferably, filter sterilization is used. Sterile filtration may include a 0.45 μm filter followed by a 0.22 μm sterilizing grade filter train. Sterilization may be carried out before or after addition of any excipients and/or carriers.

The pharmaceutical composition may be in dried, such as lyophilized, form. The composition of the invention may be an aqueous solution, for example an aqueous solution formed by dissolving a lyophilizate or other dried formulation in an aqueous medium. The aqueous solution is typically pH neutral.

Drying the formulation facilitates long-term storage. Any suitable drying method may be used. Lyophilization is preferred but other suitable drying methods may be used, such as vacuum drying, spray-drying, spray freeze-drying or fluid bed drying. The drying procedure can result in the formation of an amorphous cake within which the compound of the invention is incorporated.

For long-term storage, the sterile composition may be lyophilized. Lyophilization can be achieved by freeze-drying. Freeze-drying typically includes freezing and then drying.

Variations to the process flow are permitted, as known to one skilled in the art, to achieve the same resulting product characteristics; namely, that the input components are blended homogenously together to the desired ratios with any aggregates dispersed, rendered sterile and presented in a suitable format for administration. Such examples could include the introduction of a vortexing and/or sonication solubilization or post-dilution stage to facilitate solubilization. Other permutations of the manufacturing process flow could include sterile filtration being performed at an earlier stage of the process or the omission of lyophilization to permit a liquid final presentation.

Pharmaceutically acceptable compositions of the invention may be solid compositions. The composition may be obtained in a dry powder form. A cake resulting from lyophilization can be milled into powder form. A solid composition according to the invention thus may take the form of free-flowing particles. The solid composition typically is provided as a powder in a sealed vial, ampoule or syringe. If for inhalation, the powder can be provided in a dry powder inhaler. The solid matrix can alternatively be provided as a patch. A powder may be compressed into tablet form.

The dried, for example, lyophilized, composition may be reconstituted prior to administration. As used herein, the term “reconstitution” is understood to mean dissolution of the dried pharmaceutical composition product prior to use. Following drying, such as lyophilization, the compound preferably is reconstituted to form an isotonic, pH neutral, homogeneous suspension. The formulation is typically reconstituted in the aqueous phase, for example by adding Water for Injection, histidine buffer solution (such as 28 mM L-histidine buffer), sodium bicarbonate, Tris-HCl or phosphate buffered saline (PBS) in the presence or absence of additional ions. The reconstituted formulation is typically dispensed into sterile containers, such as vials, syringes or any other suitable format for storage or administration.

The composition may be stored in a container, such as a sterile vial or syringe, prior to use.

Methods of Use

In embodiments provided herein are compounds and pharmaceutical compositions thereof for administration to a subject, wherein the immunotherapy compounds induce and/or enhance an immunological response in the subject to a host antigen. The present compounds comprising an IM portion that comprises a TLR7 and/or TLR8 agonist that induce immunological responses by activating toll-like receptor (TLR) 7 or by activating TLR7 and TLR8. Some of the compounds of the present disclosure may also induce other immunological responses in the host in addition to the activation of TLR7 and/or TLR8, such as by stimulating interferons (IFN). In general, an “immunological response” refers to a response by the host's immune system to a stimulus. In embodiments, the administration of the present immunotherapy compounds activates the host immune system.

In embodiments, the present immunotherapy compounds are administered via IT (intratumoral) or peritumoral (PI) and induce an immune response against the tumor antigens present in the tumor. The present immunotherapy compounds may stimulate or “enhance” an immunological response to the tumor by reducing local tumor immune suppression, wherein suppressive and/or protumoral myeloid cells (such as MDSC, TAM and/or M2 cells) are converted into anti-tumor myeloid cells (M1). This can lead to the priming of an immune response against a tumor through the stimulation of infiltrating antitumor B cells, T cells or NK cells. This process allows the use of the tumor as its own vaccine by generating antitumor immunity against cancer cell antigens, wherein the present immunotherapy compounds are the stimulus that activates the host immune system leading to the antitumor immunity.

In certain embodiments provided herein are methods of inducing a cell mediated immune response in a subject. In embodiments, the methods comprise locally administering a liquid form of the present pharmaceutical composition (comprising an immunotherapy compound of the present disclosure) into the subject, wherein in vivo physiological conditions reduce solubility of the DM component of the immunotherapy compound wherein the immunotherapy compounds form insoluble self-assemblies or aggregates in vivo; whereby the insoluble self-assemblies or aggregates induce a cell mediated immune response at the local site of administration and/or at a distant site from the site of administration of the pharmaceutical composition. In embodiments, the local site of administration is intratumoral or peritumoral.

In certain embodiments, the pharmaceutical compositions are used as a vaccine to induce an anti-tumor immune response. In embodiments, such compositions include an immunotherapy compound of the present disclosure in an amount effective to treat the tumors. In certain embodiments, the pharmaceutical compositions are used as a vaccine for cancers with solid tumors. In embodiments, such compositions include an immunotherapy compound of the present disclosure in an amount effective to treat the cancers. In embodiments, the cancers are selected from head and neck cancer, ovarian cancer, breast cancer, colon cancer, colorectal cancer, lung cancer, melanoma, gastric cancer, gallbladder cancer, bladder cancer, osteosarcoma, oral cancer, pancreatic cancer, gastric cancer, Merkel-cell carcinoma, liver cancer, cervical cancer, kidney cancer, or lymphoma.

Intratumoral or peritumoral injection can be achieved using needle systems including multipronged array needle, micro-needle or micro-needle array. For deeper lesions, a 22-gauge needle may be used and for superficial lesions, needles as small as 30-gauge can be used. Intratumoral injections may be guided using imaging systems including ultrasonography, endoscopic ultrasonography, computed tomography or magnetic resonance imaging.

The administration to local mucosal body sites may also be addressed through pulmonary administration, nasal administration (e.g., intranasal), buccal administration, or intravesical administration. Locally acting TLR7 and/or TLR8 agonists may also be useful for the treatment of an airway disease. In allergic or virally-induced asthma and allergic rhinitis, TLR7 and/or TLR8 agonists would induce Th1 immunity that may attenuate the excessive Th2 phenotype but also stimulate bronchodilatation via the production of nitric oxide. A TLR7/8 agonist delivered through spray, aerosol or nebulization and forming a depot in the mucosal environment preventing its systemic release would be highly beneficial for the treatment of these airway diseases. In embodiments, the pharmaceutical compositions are used as a vaccine to induce a cell mediated immune response to treat allergic, asthma or virally-induced asthma and/or allergic rhinitis.

In embodiments, the pharmaceutical compositions of this disclosure may benefit from concurrent combination with other systemic immunotherapies such as checkpoint inhibitors, adoptive T cell transfer including TILs (tumor-infiltrating lymphocytes) or CAR-T cells, monoclonal antibodies targeting tumor cells, CD3-bi-specific antibodies or T cell receptors, or virotherapy such as oncolytic viruses or vaccine cytokines. In other embodiments, the present pharmaceutical compositions may also benefit from combination treatment with other immune stimulants administered intratumorally or systemically such as TLR2, TLR3, TLR5, TLR9, STING, cGAS or NOD agonists either administered temporally at the same time (co-formulated or not) or at different times.

In some embodiments, this disclosure provides methods for treating and/or preventing tumor growth by administration (i.e., systemic administration and/or intratumoral administration (by, e.g., injecting active agent(s) directly into a tumor)) of one or more of the peptide conjugates described herein (e.g., in preferred embodiments kHL-12) alone and/or with one or more additional anti-tumor agents such as, e.g., in preferred embodiments one or more systemic immune checkpoint inhibitors and/or are directed at one and/or more myeloid-derived suppressor cells (MDSC) inhibitor targets. In some embodiments, the peptide conjugate is selected from the group consisting of kHL-12, HL-4X2, AH-3X2, HL-6X2, HL-5X2, HL-4X3, AH-3X3, HL-6X3, HL-5X3, HL-4X4, AH-3X4, HL-6X4, and HL-5X4 and/or, as shown in FIGS. 12-13. In preferred embodiments, the immunotherapy compound is selected from the group consisting of kHL-12, HL-6X2, and HL-6X3.

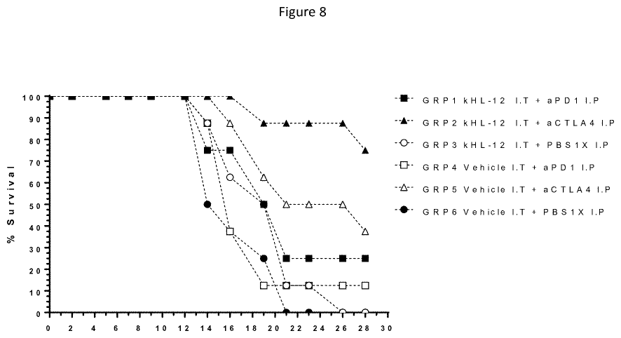

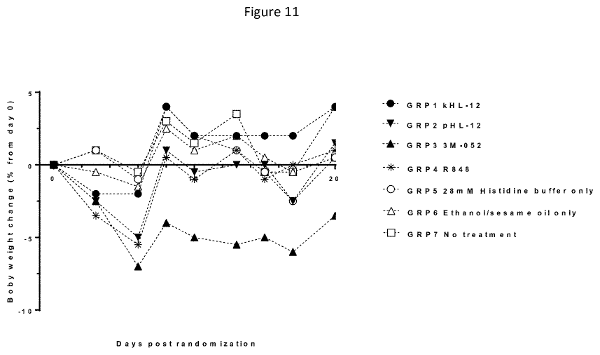

In some embodiments, one or more systemic immune checkpoint inhibitors are directed at, without limitation, PD1, PDL1, PDL2, CD28, CD80, CD86, CTLA4, B7RP1, ICOS, B7RPI, B7-H3, B7-H4, BTLA, HVEM, KIR, TCR, LAG3, CD137, CD137L, OX40, OX40L, CD27, CD70, CD40, CD40L, TIM3, GAL9, ADORA, CD276, VTCN1, IDO1, KIR3DL1, HAVCR2, VISTA or CD244. In some embodiments, the immune checkpoint inhibitor is preferably anti-PD1 and/or anti-CTLA-4, and even more preferably anti-CTLA-4, particularly for extra-tumoral administration (i.e., non-injected tumors, systemic administration of the active agents). In certain other embodiments, a myeloid-derived suppressor cell (MDSC) inhibitor targets PGE-2, COX2, NOS2, ARG1, PI3K, CSF-1R, Caspase-8, CCL2, RON, ROSS100A8/A9 or liver-X nuclear receptor. In embodiments, the MDSC inhibitor is PF-5480090, INCB7839, nitro-aspirine, SC58236, celecoxib, IPI-549, PLX3397, BLZ945, GW2580, RG7155, IMC-CS4, AMG-820, ARRY-382, sildenafil, tadalafil, vardenafil, N-hydroxy-nor-L-Arg, imatinib, z-IETD-FMK, trabectedin, emricasan, anti-CCL2 antibody (carlumab, ABN912), tasquinimod, ASLAN002, IMC-RON8, or GW3965. In preferred embodiments, such methods induce tumor regression in tumors (e.g., injected tumors) above that observed in a control group (e.g., to whom or which the peptide conjugate(s) were not administered (see, e.g., FIG. 5)). In some embodiments, the peptide conjugate and one or more additional active agents may be administered separately, and in some embodiments together (e.g., physically together and/or simultaneously administered to different anatomical sites). For instance, in some embodiments, the methods include intratumoral administration of kHL-12 combined with one or more systemic immune checkpoint inhibitors, either anti-PD1 or anti-CTLA-4, preferably anti-CTLA-4, to induce tumor stabilization and/or regression in non-injected tumors (e.g., distal tumors such as metastases, a phenomenon generally referred to abscopal effect). In some embodiments, the methods result in a surprising synergistic effect of a peptide conjugate of this disclosure (e.g., in preferred embodiments kHL-12) in combination with anti-CTLA-4 is observed (see, e.g., FIG. 8). In some embodiments, this disclosure also provides methods for inducing stabilization of tumor volume over time (an antitumoral activity) with less or without side effects (e.g., in preferred embodiments reduced body weight and/or limiting the off-target side effects leading to the induction of a deleterious systemic burst of pro-inflammatory cytokines; e.g., an improved safety profile) observed following administration of other active agents (e.g., such as 3M-052 and/or R848) (see, e.g., FIG. 11).

Thus, this disclosure provides, in some embodiments, compounds having the structure of Formula (I): DM-L-IM, wherein DM comprises a peptide from about 18 to about 45 amino acids in length comprising amino acid residues possessing helix forming properties wherein the DM is configured to form an amphipathic α-helix structure, and wherein the peptide sequence does not comprise a T cell epitope and/or a B cell epitope and is a non-natural sequence; wherein L is a linker; and, IM is a toll-like receptor 7 (TLR7) and/or TLR8 agonist selected from Formulas (Ia) through (Im), wherein: R₂ is selected from —CH₃, —CH₂CH₃, —CH₂CH₂CH₃, —CH₂CH(CH₃)₂, —CH₂CH₂CH₂CH₃, —CH₂CH₂CH₂CH₂CH₃, —CH₂OCH₂CH₃, —CH₂CH₂OCH₃, —CH₂NHCH₂CH₃, and —CH₂Ph; and R comprises the linker connecting the IM to an amino group or carboxyl group of the peptide at the peptide termini or the lateral chain of an amino-acid such as lysine or glutamine, wherein the linker is -[A1]—NH—, and A1 is selected from: -A2-A3-(CH₂)ₓ—CO—, -A2-A3-CH₂—O—CH₂—CO—, -A2-A3-(CH₂)ₓ—O—(CH₂)ₓ—O—(CH₂)ₓ—O—(CH₂)ₓ—CO—, -A2-Valine-Alanine-A4-, -A2-Valine-Citrulline-A4-, -A2-Glutamate-Valine-Citrulline-A4-, or -A2-Phenylalanine-Lysine-A4-; wherein: A2 is selected from: -A5-(CH₂)ₓ-A6-, -A5-(CH₂)ₓ—O—(CH₂)ₓ—O—(CH₂)ₓ—O—(CH₂)ₓ-A3-, -A5-(CH₂)₂—(O—CH₂—CH₂)ₓ-A6-, -A5-CH₂—O—CH₂-A6-, -A5-(CH₂)ₓ-A6-, -A5-(CH₂)ₓ—O—(CH₂)ₓ—O—(CH₂)ₓ—O—(CH₂)ₓ-A6-, or, -A5-NH—(CH₂)₂—O—(CH₂)-A6-; A3 is —CO— or —NH—; A4 is p-aminobenzyloxy carbonyl (PABC) or nothing; A5 and A6 are —CO— or —NH—, one or more natural or non-natural amino-acids, or nothing; B is selected from 0 and NH; m is any integer from 1 to 11; and, x is any integer from 1 to 12, or wherein x is any integer from 2 to 12. In some embodiments, the IM is derived from or comprises a compound of Formula 15 described herein wherein R₂ is an alkyl optionally selected from the group consisting of CH₂CH₂CH₂CH₃, CH₂OCH₂CH₃, CH₂CH₂OCH₃, CH₂NHCH₂CH₃, CH₃, CH₂CH₃, CH₂CH₂CH₃, CH₂CH(CH₃)₂, CH₂CH₂CH₂CH₂CH₃, CH₂Ph, and CH₂NCbzCH₂CH₃, such as any of compounds 15a through 15j. In some embodiments, the DM further comprises a hydrophobic moiety covalently attached to a terminal amino acid of the peptide which can be, in some embodiments, the hydrophobic moiety is selected from C₈F₁₇—(CH₂)₂—CO—, CH₃(CH₂)₁₂CO—, CH₃(CH₂)₁₄CO—, CH₃(CH₂)₁₆CO—, or

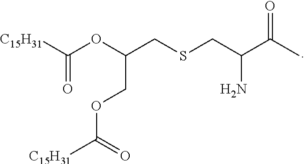

In some embodiments, the peptide has less than 70% sequence identity with a bacterial, fungal or viral antigen or immunogen. In some embodiments, the peptide comprises an amino acid sequence of RRLL(5)A(7)LAL(11)A(13)LLRRL (SEQ ID NO: 1) wherein amino acid positions (5), (7), (11) and (13) are each selected from A, L, or H. In some embodiments, the peptide comprises an amino acid sequence selected from RRLLHAHLALHAHLLRRLK (SEQ ID NO: 2), RRLLAAHLALHAALLRRLK (SEQ ID NO:3), or RRLLHALLALLAHLLRRLK (SEQ ID NO:4). In some embodiments, the compound is a peptide conjugate. In some embodiments, the peptide conjugate is selected from the group consisting of HH-12, AH-12, HL-12, kHL-12, kAH-12, pHL-12, pAH-12, where Pam is palmitoyl; Ac is acetyl; and, ADJ12 is derived from Formula I(a) where R is —NH—CO—CH₂—O—CH₂—CO—NH—((CH₂)₂O)₃—(CH₂)₂—COOH, or is

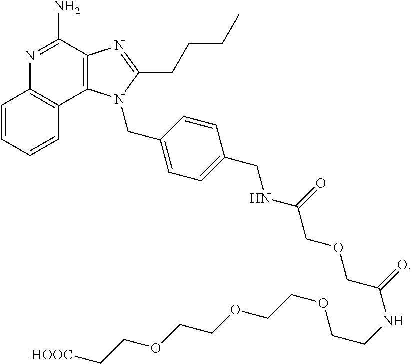 [SMILES: CCCCc1nc2c(N)nc3ccccc3c2n1Cc1ccc(CNC(=O)COCC(=O)NCCOCCOCCOCCC(=O)O)cc1]

In some embodiments, the compound is selected from the group consisting of kHL-12, HL-4X2, AH-3X2, HL-6X2, HL-5X2, HL-4X3, AH-3X3, HL-6X3, HL-5X3, HL-4X4, AH-3X4, HL-6X4, and HL-5X4 and/or, as shown in FIGS. 12-13. In preferred embodiments, the compound is selected from the group consisting of kHL-12, HL-6X2, and HL-6X3. In preferred embodiments, the immunotherapy compound is selected from the group consisting of kHL-12, HL-6X2, and HL-6X3. In some embodiments, this disclosure provides pharmaceutical compositions comprising one or more of such compounds and at least one pharmaceutically acceptable carrier and/or diluent (e.g., in some embodiments, a buffer to adjust solubility of the compound where the buffer can comprise comprises at least one of a salt, amino acid and/or sugar compound, at least one of sodium chloride, phosphate, citrate, succinate, acetate, benzoate, carbonate, bicarbonate, tris, mannitol, sorbitol, inositol, sucrose, trehalose, dextrose, glucose, lactose, maltose povidone, histidine, methionine, arginine or a combination thereof, and/or at least one surfactant or preservative). In some embodiments, the compound is insoluble at physiological conditions. In some embodiments, the compound is soluble in an aqueous solution having a pH range aqueous solution having a pH range of about 3 to 9 (in embodiments, about 4 to 8) or an ion concentration ranging from 0 mM to 600 mM (in some embodiments about 400 mM). In some embodiments, the pharmaceutical composition(s) can further comprise at least one systemic checkpoint inhibitor (e.g., an anti-PD1 and/or anti-CTLA-4 antibody). In some embodiments, this disclosure provides methods for inducing a cell mediated immune response in a subject, wherein the method comprises: locally administering a liquid form of the pharmaceutical composition(s) into the subject, wherein in vivo physiological conditions reduce solubility of the DM component of the immunotherapy compound wherein the immunotherapy compounds form insoluble self-assemblies or aggregates in vivo; whereby the insoluble self-assemblies or aggregates induce a cell mediated immune response at the local site of administration. In some embodiments, the local site of administration is intratumoral or peritumoral, the administration is an injection, the local site of administration is mucosal (e.g., pulmonary, nasal, intranasal, buccal and intravesical). In some embodiments, such methods can further comprise administering at least one systemic checkpoint inhibitor (e.g., anti-PD-1 and/or anti-CTLA-4 antibody). In some embodiments, this disclosure provides methods for stimulating a systemic anti-tumor immune response in a subject, comprising locally administering intratumorally or peritumorally a liquid form of such pharmaceutical composition(s) into the subject, wherein the anti-tumor immune response is effective at a distant site from the site of administration of the pharmaceutical composition. In some embodiments, such methods can further comprise administering at least one systemic checkpoint inhibitor (e.g., an anti-PD-1 and/or anti-CTLA-4 antibody). Other embodiments are also contemplated herein, as would be understood by those of ordinary skill in the art.

### Examples

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade (° C.). It should be understood that variations in the methods as described can be made without changing the fundamental aspects that the Examples are meant to illustrate.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value.

### Example 1: Development of the Delivery Moiety (DM) of the Immunotherapy Compound for Intratumoral (IT) Localized/Depot Delivery

Provided herein are DM (delivery/depot moiety) components of the immunotherapy compounds with tunable solubility, wherein the DM and its conjugate in the form of the immunotherapy compound can be soluble in an aqueous solution (such as a pharmaceutical aqueous formulation for administration) but become insoluble under physiological pH and ionic strength conditions. Hence, once administered in vivo the immunotherapy compounds form aggregates or supramolecular structures creating a depot of the immunotherapy compounds at the site of administration (e.g. intratumoral) that is maintained for a sufficient amount of time to induce a cell mediated immune response and prevent or reduce the risk of systemic release and stimulation of cytokine release syndrome. See FIG. 1. In embodiments, the present immunotherapy compound, due to the DM component, is highly soluble in a pharmaceutically acceptable solution with a low ionic strength or pH. Changes in the pH and/or ionic strength conditions found in vivo augments the hydrophobicity of the present immunotherapy compound, due to the DM component. In embodiments, the present immunotherapy compounds possess self-depot forming properties that result in the formation of immunotherapy aggregates that reside at the site of administration.

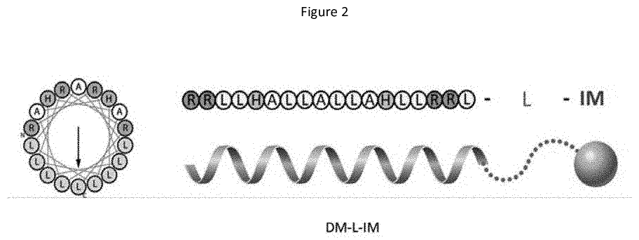

In embodiments, the DM comprises a peptide from about 17 to about 45 amino acids in length comprising amino acid residues possessing helix forming properties wherein the DM is configured to form an amphipathic α-helix structure. See FIG. 2. It is understood the peptide is an artificial palindromic sequence that is non-native (and non-natural) and not derived from (e.g. does not have significant homology or identify with) an antigen or immunogen relevant for immunotherapy. It is understood that this peptide is not designed to be recognized by human T cells or human antibodies from healthy or diseased patients.

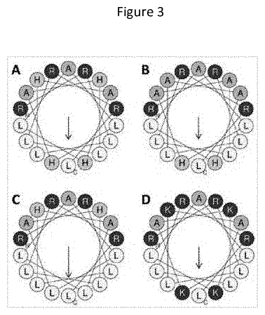

Peptides were rationally designed based on principles governing the folding of an amphiphile α-helix presenting a hydrophobic and hydrophilic face. The peptides incorporate amino acids possessing helix-forming properties (e.g. alanine, leucine and arginine), devised to preserve the alpha-helical periodicity with 3.6 amino-acids per turn while ensuring a balance between hydrophobic and charged residues. A palindromic pattern along the peptide sequence was engineered with alanine and leucine residues positioned towards the central part of the peptides and arginine residues positioned on both extremities. See FIG. 3.

A range of peptide sequence were selected based on alteration of their physicochemical properties through the modification of positions 5, 7, 11 and/or 13 of the peptides by alanine, leucine or histidine residues. In embodiments, the present peptides comprise the following sequence RRLL(5)A(7)LAL(11)A(13)LLRRL (SEQ ID NO: 1), wherein amino acid positions (5), (7), (11) and (13) are each selected from A, L, or H. See Table 1.

TABLE 1
Selected peptide sequences

| Sequences | SEQ ID NO: 2 to 8 |
|---|---|
| RRLLHAHLALHAHLLRRLK | SEQ ID NO: 2 |
| RRLLAAHLALHAALLRRLK | SEQ ID NO: 3 |
| RRLLHALLALLAHLLRRLK | SEQ ID NO: 4 |
| RRLLKAKLALKAKLLRRLK | SEQ ID NO: 5* |
| KRRLLHALLALLAHLLRRLK | SEQ ID NO: 6 |
| KRRLLAAHLALHAALLRRLK | SEQ ID NO: 7 |
| RRLLHALLALLAHLLRRLE | SEQ ID NO: 8 |

*negative control

The response to stimuli such as pH and ion concentration is essentially provided by the presence of the histidine residues. Histidine is a unique amino acid because the pKa of its imidazole side chain is close to physiological pH. The pKa of histidine residues vary between 5.5 to 7.2 depending on the position in the peptide sequence. Moreover, the contribution of histidine to the peptide alpha-helicity is strongly dependent upon its charged state, with the uncharged state having a higher contribution to the alpha-helicity. In addition, as the histidine residue become uncharged when the pH is above its pKa, its hydrophobicity also increases. Based on those principle, three peptides named AH (SEQ ID NO: 3), HL (SEQ ID NO: 4), HH (SEQ ID NO: 2) comprising SEQ ID NO:1 and a negative control peptide KK (SEQ ID NO: 5 (outside the definition of SEQ ID NO:1)) were selected based on their different physicochemical properties.

Primary sequences and helical wheel representations are presented in FIG. 3. Peptide KK contains lysine residues at position 5, 7, 11 and 13. Peptide KK was designed as a negative control peptide. Peptide KK is closely related to sequence AH, HL and HH but does not comprise A, L, or H residues at amino acid positions (5), (7), (11) and (13) of SEQ ID NO:1. Peptide KK was selected for its anticipated absence of significant physicochemical response to change in pH and ion concentration. Peptide HH contains histidine residues at position 5, 7, 11 and 13 exposing them on both the hydrophobic and hydrophilic face of the helix. Peptide HL contains histidine residues at position 5 and 13 corresponding to the hydrophilic face of the helix. Peptide AH contains histidine residues at position 7 and 11 corresponding to the hydrophobic face of the helix.

### Example 2: Preparation of Immunotherapy Compounds Having the Structure of Formula (I): DM-L-IM (Delivery Moiety-Linker-Immunostimulant Moiety)

Provided herein are oncology therapy compounds comprising a Delivery/Depot Moiety (DM) covalently attached to an immunostimulant moiety (IM) via a Linker (L). In embodiments, the IM is a TLR7 and/or TLR 8 agonist. See Example 7 for synthesis of multiple immunostimulant moieties (e.g., TLR7/8 agonists) that can be used in the present immunotherapy compounds and conjugated to the present DM.

The synthesis of the peptide conjugates provided in Table 2 was performed on solid phase using a classical Fmoc strategy. A functionalized imidazoquinoline moiety named ADJ12 corresponding to the small molecule immune stimulant of Formula (Ia) where R is NH—CO—CH₂—O—CH₂—CO—NH—(CH₂)₂—O—(CH₂)₂—O—(CH₂)₂—O—(CH₂)₂—COOH) was conjugated to the epsilon side chain of a C-terminal Lysine. A final cleavage in the presence of TFA and a subsequent RP-HPLC purification gave access to the peptide conjugates with purity >90%. See Tables 2A-C for each of the four present immunotherapy compounds that were prepared. ADJ12 is shown below:

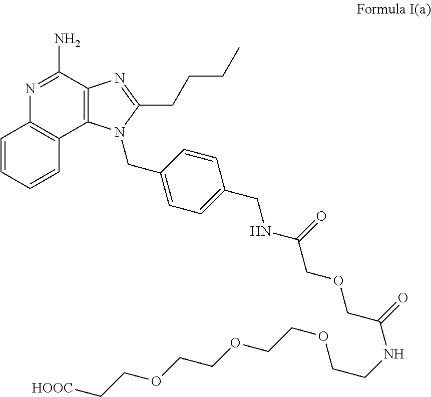

where R is —NH—CO—CH₂—O—CH₂—CO—NH—((CH₂)₂O)₃—(CH₂)₂—COOH. Certain of these exemplary peptide conjugates and derivatives thereof were tested in vivo as described below.

TABLE 2A
Selected Immunotherapy Compounds with
Formula (Ia) as the IM

| Peptide | | Peptide |
|---|---|---|
| conju- | | SEQ ID |
| gates | Sequences | NO. |
| HH-12 | Ac-RRLLHAHLALHAHLLRRLK(ADJ12)-NH₂ | 2 |
| AH-12 | Ac-RRLLAAHLALHAALLRRLK(ADJ12)-NH₂ | 3 |
| HL-12 | Ac-RRLLHALLALLAHLLRRLK(ADJ12)-NH₂ | 4 |
| KK-12 | Ac-RRLLKAKLALKAKLLRRLK(ADJ12)-NH₂ | 5 |

Ac = Acetyl; ADJ12 = Formula (Ia) where R is NH—CO—CH₂—O—CH₂—CO—NH—(CH₂)₂—O—(CH₂)₂—O—(CH₂)₂—O—(CH₂)₂—CO—; HH, AH, HL and KK are each the DM component of the immunotherapy compound. See also FIG. 12.

Additional exemplary conjugates comprising the peptide sequences of SEQ ID NOS. 6 and 7 (each comprising SEQ ID NO: 1) conjugated to the immune stimulant compound ADJ12 were also produced as described in Table 2B. These exemplary peptide conjugates were synthesized by solid phase using a classical Fmoc strategy. A final cleavage in the presence of TFA and a subsequent RP-HPLC purification gave access to expected peptide conjugates. The lyophilized peptide conjugates were obtained with a purity >90% and stored in the freezer.

TABLE 2B
Selected Immunotherapy Compounds with
ADJ12 as the IM

| | | Peptide |
|---|---|---|
| | | SEQ |
| Name | Structure | ID NO. |
| kHL-12 | K(Ac)- | 6 |
| | RRLLHALLALLAHLLRRLK(ADJ12)-NH₂ | |
| kAH-12 | K(Ac)- | 7 |
| | RRLLAAHLALHAALLRRLK(ADJ12)-NH₂ | |
| pHL-12 | K(Pam)- | 6 |
| | RRLLHALLALLAHLLRRLK(ADJ12)-NH₂ | |
| pHL-12 | K(Pam)- | 7 |
| | RRLLAAHLALHAALLRRLK(ADJ12)-NH₂ | |

Pam—Palmitoyl
Ac—Acetyl
ADJ12 comprising Formula (Ia) where R is —NH—CO—CH₂—O—CH₂—CO—NH—(CH₂)₂—O—(CH₂)₂—O—(CH₂)₂—O—(CH₂)₂—CO—

Further exemplary conjugates comprising the peptide sequences of SEQ ID NO: 8 (comprising SEQ ID NO:1) conjugated to different immunostimulant moieties comprising cleavable linkers were also produced as described in Table 2C. These exemplary peptide conjugates are synthesized by solid phase using a classical Fmoc strategy. A final cleavage in the presence of TFA and a subsequent RP-HPLC purification gave access to expected peptide conjugates. The lyophilized peptide conjugates were obtained with a purity >90% and stored in the freezer.

TABLE 2C
Selected Immunotherapy Compounds with cleavable linkers

| | | Peptide SEQ ID |
|---|---|---|
| Name | Structure | NO. |
| HL-4X2 | Ac-RRLLHALLALLAHLLRRLE(Val-Cit-PABC-IMDQ)—NH₂ | 8 |
| | 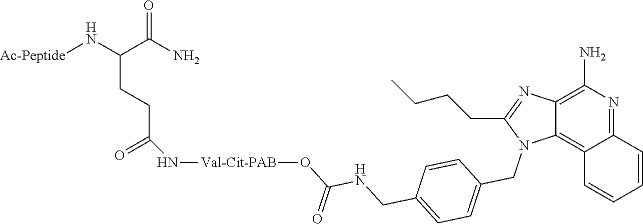 | |
| AH-3X2 | Ac-RRLLHALLALLAHLLRRLE(Val-Cit-IMDQ)—NH₂ | 8 |
| | 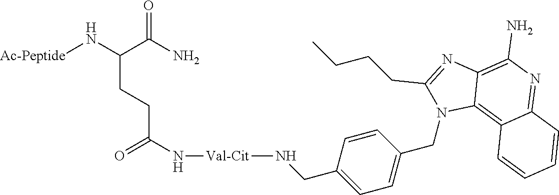 | |
| HL-6X2 | Ac-RRLLHALLALLAHLLRRLE(PEG₆-Val-Cit-PABC-IMDQ)—NH₂ | 8 |
| | 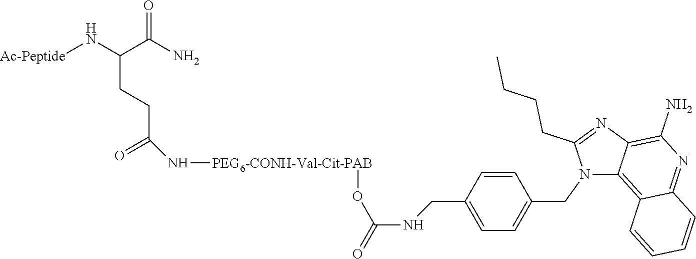 | |
| HL-5X2 | Ac-RRLLHALLALLAHLLRRLE(PEG₆-Val-Cit-IMDQ)—NH₂ | 8 |
| | 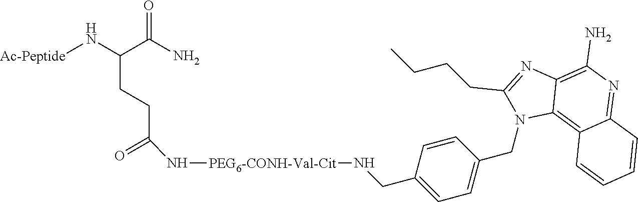 | |
| HL-4X3 | Ac-RRLLHALLALLAHLLRRLE(Val-Cit-PABC-IM3)—NH₂ | 8 |
| | 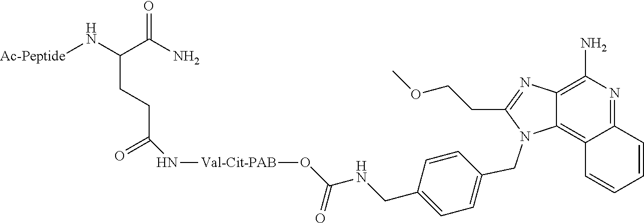 | |
| AH-3X3 | Ac-RRLLHALLALLAHLLRRLE(Val-Cit-IM3)—NH₂ | 8 |
| | 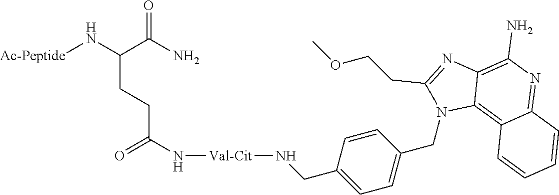 | |
| HL-6X3 | Ac-RRLLHALLALLAHLLRRLE(PEG₆-Val-Cit-PABC-IM3)—NH₂ | 8 |
| | 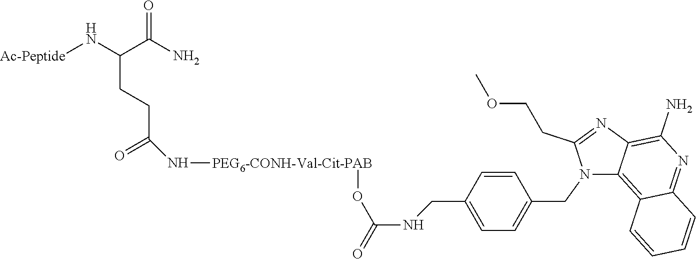 | |
| HL-5X3 | Ac-RRLLHALLALLAHLLRRLE(PEG₆-Val-Cit-IM3)—NH₂ | 8 |
| | 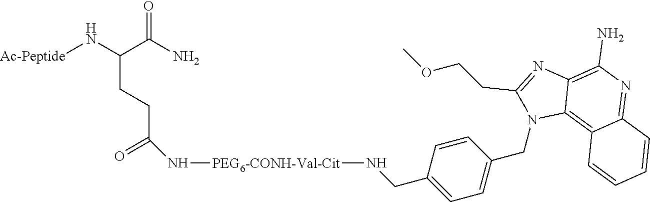 | |
| HL-4X4 | Ac-RRLLHALLALLAHLLRRLE(Val-Cit-PABC-IM4)—NH₂ | 8 |
| | 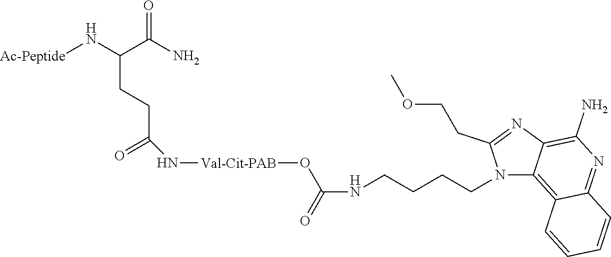 | |
| AH-3X4 | Ac-RRLLHALLALLAHLLRRLE(Val-Cit-IM4)—NH₂ | 8 |
| | 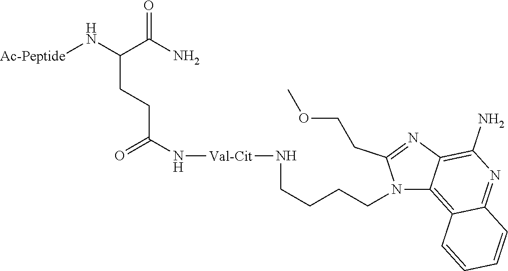 | |
| HL-6X4 | Ac-RRLLHALLALLAHLLRRLE(PEG₆-Val-Cit-PABC-IM4)—NH₂ | 8 |
| | 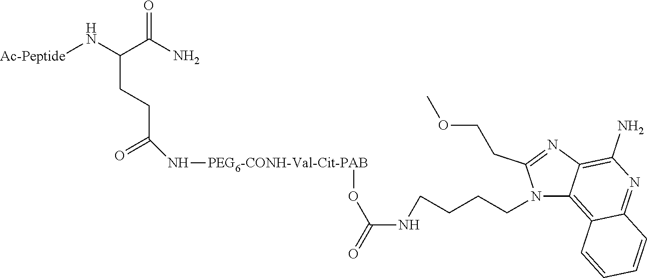 | |
| HL-5X4 | Ac-RRLLHALLALLAHLLRRLE(PEG₆-Val-Cit-IM4)—NH₂ | 8 |
| | 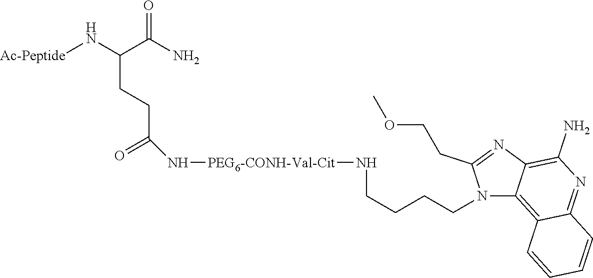 | |

Ac-Acetyl
Val-Valine
Cit-Citrulline
PEG₆-—NH—(CH₂)₂—(O—CH₂—CH₂)₆—CO—
PABC-p-aminobenzyloxy carbonyl
PAB-p-aminobenzyloxy
IMDQ-Formula (Ia) where R is —NH—
IM3-Formula (Ik) where R is —NH—
IM4-Formula (Ii) where R is —NH—

### Example 3: Evaluation of Secondary Structure of Different Immunotherapy Compounds (Peptide Conjugates)

The secondary structure of peptide conjugates KK-12, HH-12, AH-12 and HL-12 was evaluated by circular dichroism (CD) in different buffer conditions. CD experiments were followed with a Jasco J-815 spectropolarimeter (Easton, Md.) fitted with an automatic 6-position Peltier thermostated cell holder. The instrument was calibrated with 10-camphorsulphonic acid. Far-UV CD data were obtained using a 0.1 mm path length cell (Quartz-Suprasil, Hellma UK Ltd.) at 20° C.±0.1° C. or 37° C.±0.1° C. Spectra were acquired using a continuous scan rate of 50 nm/min and averaging at least ten scans between 180 to 260 nm. Each spectrum was carried out in 10 mM tris (pH 7.5) and sodium acetate 18 mM (pH 5.2) in the presence or absence of sodium chloride 154 mM. Peptide conjugate solution were prepared at a concentration of 240 μmol/ml. All spectra were corrected by subtraction of the corresponding solvent spectrum containing 240 μmol/ml of the ADJ12 (Formula (Ia) where R is NH—CO—CH₂—O—CH₂—CO—NH—(CH₂)₂—O—(CH₂)₂—O—(CH₂)₂—O—(CH₂)₂—COOH) in the corresponding buffer conditions. The signal is expressed in mean residue ellipticity (deg.cm2.dmol−1). The percentage α-helices, β-sheets and other structures obtained after deconvolution of the CD curves are presented in Table 3.

TABLE 3
Secondary structure of the peptide conjugates in different buffer conditions

| | | pH 5.2 | | | | pH 7.5 | | | |
|---|---|---|---|---|---|---|---|---|---|
| | | — | | NaCl 154 mM | | — | | NaCl 154 mM | |
| | | 20° C. | 37° C. | 20° C. | 37° C. | 20° C. | 37° C. | 20° C. | 37° C. |
| KK-12 | ALPHA | 23% | 26% | 41% | 38% | 24% | 29% | 45% | 39% |
| | BETA | 9% | 11% | 7% | 7% | 8% | 9% | 3% | 6% |
| | OTHERS | 69% | 63% | 52% | 55% | 68% | 62% | 53% | 56% |
| | NRMSD | 4% | 3% | 6% | 4% | 3% | 4% | 3% | 6% |
| HH-12 | ALPHA | 9% | 17% | 16% | 19% | 41% | 37% | 46% | 39% |
| | BETA | 17% | 14% | 24% | 22% | 7% | 8% | 8% | 13% |
| | OTHERS | 74% | 69% | 61% | 60% | 52% | 55% | 46% | 48% |
| | NRMSD | 2% | 5% | 9% | 9% | 4% | 3% | 3% | 10% |
| AH-12 | ALPHA | 34% | 32% | 43% | 39% | 59% | 50% | 81% | 77% |
| | BETA | 11% | 12% | 6% | 8% | 4% | 5% | 1% | 8% |
| | OTHERS | 56% | 56% | 52% | 53% | 37% | 45% | 17% | 16% |
| | NRMSD | 4% | 4% | 6% | 5% | 3% | 3% | 1% | 3% |

| | | | | | | | | |
|---|---|---|---|---|---|---|---|---|
| HL-12 | ALPHA | 88% | 87% | 100% | 99% | 96% | 95% | Precipitated |
| | BETA | 1% | 1% | 0% | 1% | 0% | 0% | Not analyzed |
| | OTHERS | 11% | 11% | 0% | 0% | 4% | 5% | |
| | NRMSD | 2% | 1% | 2% | 2% | 2% | 1% | |

The change in pH from pH 5.2 to pH 7.5 dramatically increased the alpha-helicity for peptide conjugates HH-12 and AH-12 that both contain histidine residues. This response to change in pH is also observed in the presence of 154 mM of sodium chloride. This is in contrast with the control peptide conjugate KK-12 that does not contain histidine residues. Surprisingly, the conjugate HL-12 already achieves a very high helicity at low pH. For this peptide, in the presence of sodium chloride, a dramatic change is observed as the pH increase from 5.2 to 7.5 resulting in the precipitation of the peptide. For the four peptides (e.g. DM) under the different buffer conditions, no significant change in the secondary structure of the peptides was observed at the two temperatures tested.

### Example 4: Evaluation of Appearance and Solubility of Different Immunotherapy Compounds (Peptide Conjugates)

The solubility of peptide conjugates KK-12, HH-12, AH-12 and HL-12 was determined by visual inspection of the solution and by RP-HPLC. Twenty (20) minutes after dispersion of the peptide conjugates in different buffer conditions, the solutions were centrifuged for 10 minutes and the supernatant analyzed by RP-HPLC. The peak area of each peptide conjugate was compared with the area of the same compound made fully soluble in 10% acetic acid. Appearance of the solution was evaluated visually according to the following criteria: clear, very slightly opalescent, slightly opalescent, opalescent or precipitated. Results are presented in Table 4.

TABLE 4
Appearance and solubility of the peptide
conjugates in different buffer conditions

| | pH 5.2 | | pH 7.5 | |
|---|---|---|---|---|
| | — | NaCl 154 mM | — | NaCl 154 mM |
| KK-12 | Clear | Clear | Clear | Clear |
| | solution | solution | solution | solution |
| | >98% HPLC | >98% HPLC | >98% HPLC | >98% HPLC |
| | solubility | solubility | solubility | solubility |
| HH-12 | Clear | Clear | Clear | Very slightly |
| | solution | solution | solution | opalescent |
| | >98% HPLC | >98% HPLC | >98% HPLC | 55% HPLC |
| | solubility | solubility | solubility | solubility |
| AH-12 | Clear | Clear | Clear | Clear solution |
| | solution | solution | solution | 90% HPLC |
| | >98% HPLC | >98% HPLC | >98% HPLC | solubility |
| | solubility | solubility | solubility | |
| HL-12 | Clear | Clear | Clear | Precipitated |
| | solution | solution | solution | 0% HPLC |
| | >98% HPLC | >98% HPLC | >98% HPLC | solubility |
| | solubility | solubility | solubility | |

While peptide conjugate KK-12 remains soluble under the different buffer conditions, compounds HH-12, AH-12 and HL-12 show a change in appearance and/or HPLC solubility as a function of change in pH and/or the presence of sodium chloride. Surprisingly, peptide conjugate HL-12 is dramatically influenced by the increase in pH from 5.2 to 7.5 in the presence of sodium chloride making the peptide fully insoluble through the formation of a precipitate. As demonstrated in the next example, the immunotherapy compounds with the highest insolubility, i.e., precipitated in pH 7.5 solution in the presence of sodium chloride, (e.g. HL-12) demonstrated the highest level of cell mediated immunological activity at the site of administration.

### Example 5: Evaluation of In Vivo Immunological Activity of Different Oncology Therapy Compounds (Peptide Conjugates) in Mice

### Example 5A: In Vivo Evaluation of Exemplary Peptide Conjugates KK-12, HH-12, AH-12 or HL-12

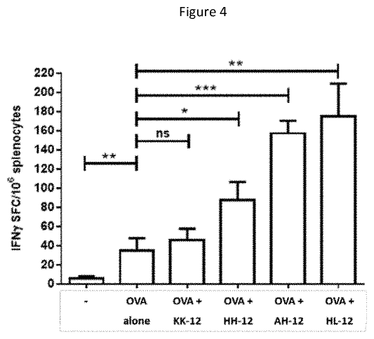

The immunological activities in vivo of peptide conjugates KK-12, HH-12, AH-12 or HL-12 were assessed after subcutaneous administration in C57BL/6J mice. Six to eight-week-old female mice (n=8/per group, Charles River) received 50 μL of co-formulation comprising the 10 nmol of different peptide conjugates and 2 nmol of ovalbumin (OVA) on day 0 and day 14 in 28 mM Histidine buffer at pH 7. Control groups (n=8/per group) received either 2 nmol of ovalbumin in 50 μL of 28 mM Histidine buffer at pH 7 or 50 μL of 28 mM Histidine buffer at pH 7 on day 0 and day 14. Ten days after the last administration, suspensions of splenocytes were prepared. Splenocytes were washed, counted and resuspended in complete media (RPMI GlutaMAX™, Invitrogen), 10% fetal calf serum, 10 g/mL gentamicin, sodium pyruvate and β-mercaptoethanol prior to incorporation in the IFNγ ELIspot assay immunological assays. PVDF plates (Millipore) were coated overnight at 4° C. with rat anti-mouse IFNγ antibody (BD Biosciences) and blocked for 1 hour with complete media. Splenocytes were applied to plates at 5×10⁵ cell/well and were re-stimulated with ovalbumin at 1 0 μg/ml, complete media alone as a negative control and 0.25 μg/mL concanavalin A as positive control. After 18 hours of culture in a 5% CO₂ incubator at 37° C., plates were washed and incubated with biotinylated rat anti-mouse IFNγ followed by Streptavidin-HRP (BD Biosciences). Spots were visualized with AEC substrate and quantified using an automated ELISpot reader system (CTL). Results expressing spot forming cells (SFC) per million splenocytes are presented in FIG. 4.

Peptide conjugate KK-12 shows an immunological activity that does not significantly differ from the condition where no peptide conjugate was used (OVA alone). By contrast, HH-12, AH-12 and HL-12 show significant improvement of their in vivo immunological activity compared to the OVA alone group. These results are likely to reflect the respective ability of the peptide physicochemical properties to change because of their exposure to physiological conditions. More specifically, compounds HL-12 and AH-12, which tend to have a high helicity and/or form insoluble material when exposure to physiological pH in the presence or absence of sodium chloride (as described in the examples above), achieved the highest in vivo immunological activity, a result that contrasts with peptide conjugate KK-12.

This experiment demonstrates the ability of TLR7/8 agonist peptide conjugates (present immunotherapy compounds) to stimulate cell mediated immune responses to an antigen present at the injection site (here co-delivered with the immune stimulant for simplification). This result would suggest that any antigen present at the injection site such as self-antigen, tumor-associated antigen, neoantigens, or neoepitopes could also stimulate a cell mediated immune response benefiting from the co-localization with TLR7/8 agonist peptide conjugate and its associated local immune activity. It is understood that such a mechanism is likely to occur if the TLR7/8 agonist peptide conjugate is administered intratumorally.

### Example 5B: In Vivo Evaluation of Exemplary Peptide Conjugate kHL-12 and Selected Derivatives Thereof

1. Synergy with Immune Checkpoint Inhibitors

The synergy between the intratumoral treatment with kHL-12 and immune checkpoint inhibitors was examined in the CT26 colon carcinoma tumor in BALB/c mice. Six to eight-week-old female BALB/c mice (Charles River) received 2×10⁵ CT26 cells subcutaneously on both the right and the left flank. When the largest tumors on either the left or right side reached an average volume of ˜100 mm³, mice were randomized into six groups of eight mice per group, approximately 10 days post tumor cell grafting, referred as day 0. After randomization, tumor size and body weight were assessed three times a week for the duration of the study. Animals were sacrificed according to the following humane endpoints: combined tumor volumes ≥3000 mm³, presence of necrotic or ulcerated tumor, impaired mobility including transient prostration or hunched posture, or interference with a vital physiological function. On day 1, only the largest tumors were intratumorally treated with 50 μl of injectable solution of kHL-12, 100 nmol/animal, for groups 1, 2 and 3 or 28 mM L-Histidine/Mannitol dilution buffer as vehicle control for groups 4, 5 and 6. All mice received three intratumoral injections once every two days, on day 1, day 3 and day 5. 100 μl of an anti-PD-1 antibody concentrated at 2 mg/ml in PBS-1X (IgG2a, clone RMP1-14, Bioxcell reference BP0146) for groups 1 and 4 or an anti-CTLA4 antibody concentrated at 2 mg/ml in PBS-1X (IgG2b, clone 9H10, Bioxcell reference BP0131) for groups 2 and 5 or PBS-1X as control for groups 3 and 6, were administered intraperitoneally six times once every three days, on day 1, 4, 7, 10, 13 and 16.

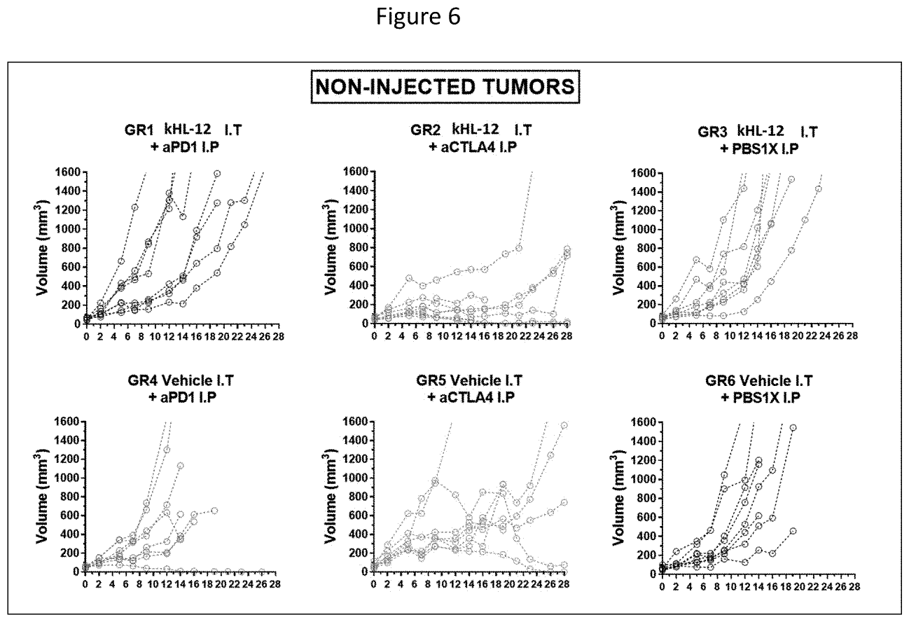

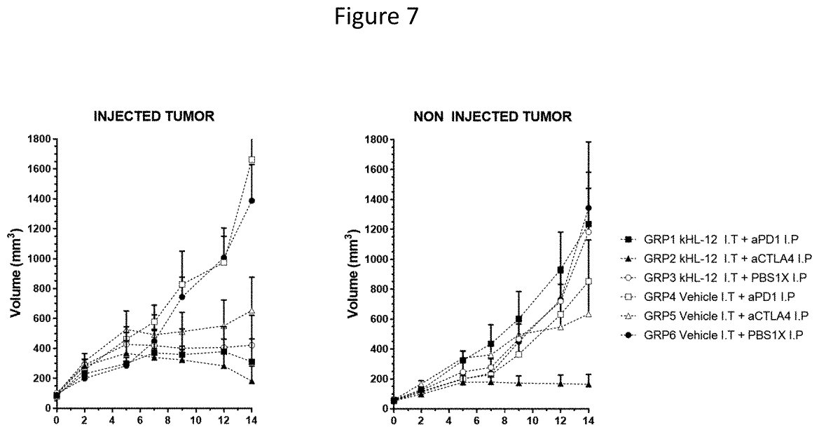

Individual tumor measurements in each group are presented in FIG. 5 (injected tumor) and FIG. 6 (non-injected tumor). Mean tumor measurements for each group up to day 14 (latest time point for which all animals were alive) are presented in FIG. 7. Survival curves for each group are presented in FIG. 8.

Results show that intratumoral administration of kHL-12 alone (group 3) induces tumor regression in the injected tumors in a larger proportion of animals compared with the control group 6 (FIG. 5). This phenomenon appears further enhanced when intratumoral administration of kHL-12 is combined with systemic immune checkpoint inhibitors, either anti-PD1 or anti-CTLA-4, administrated intraperitoneally, (groups 1 and 2, respectively) compared to anti-PD1 and anti-CTLA-4 alone (groups 4 and 5, respectively) (FIG. 5). Treatment with kHL-12 in combination with anti-CTLA-4 (group 2) shows a higher proportion of animals showing evidence of tumor stabilization and/or regression in the non-injected tumors compared to group 5 receiving anti-CTLA-4 alone (FIG. 6). This clearly indicates the ability of the immune response primed in the injected tumor to distally affect untreated tumors, a phenomenon generally referred to as the abscopal effect. At the group level as presented in FIG. 7, treatment with kHL-12 alone (group 3) or in combination with anti-PD1 (group 1) and anti-CTLA-4 (group 2) show more significant anti-tumor activity in the injected tumor compared to the other groups that have received anti-PD1 alone (group 4), anti-CTLA-4 alone (group 5) or no active treatment (group 6). Only the combination of kHL-12 with anti-CTLA-4 shows a dramatic impact on tumor growth compared to all other groups in the non-injected tumors. These effects observed at tumor level are reflected in terms of overall survival where synergistic effect of kHL-12 in combination with anti-CTLA-4 is observed (FIG. 8).

### Example 6: Safety Profile of Oncology Therapy Compounds (Peptide Conjugates) Compared to Unmodified IM (TLR7 and/or TLR8 Agonist)

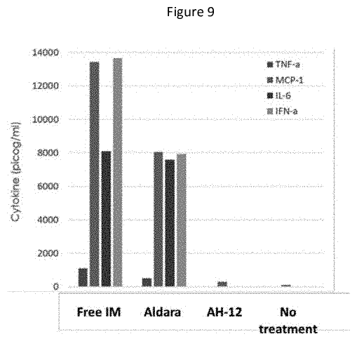

Six to eight-week-old female C57BL/6J mice (Charles River) were used to assess the ability of different treatment groups to stimulate systemic proinflammatory cytokines responses as a measure of in vivo toxicity. C57BL/6 mice (n=8/group) were subcutaneously administered with 10 nmol of the free IM (TLR7/8 agonist; formula (Ia)-NH₂) in 28 mM Histidine pH 7 or 10 nmol of AH-12 peptide in 28 mM Histidine pH 7. Another group of eight C57BL/6 mice were also topically treated with Aldara 5% (a commercial product containing imiquimod, a TLR7 agonist) on the skin of shaved animals. The negative control group (n=8) received no treatment. Serum was collected 2 hours after administration. Serum cytokine measurement was performed using a mouse cytokine multiplex kit. Sera were transferred to appropriate microtiter wells containing diluted antibody-coated bead complexes and incubation buffer. 50 μl of each homogenate sample was transferred to appropriate wells containing diluted antibody-coated bead complexes and incubation buffer. Samples were incubated for 2 hours. After washing with assay buffer (200 μl/well), 100 μl diluted biotinylated secondary antibody was added to the appropriate wells and incubated for 1 hour. After washing, 100 μl Streptavidin-phycoerythrin was added to each well and incubated for 30 minutes. After a final wash, the plate was evaluated using the Luminex analyzer (Luminex Corp., Austin, Tex.). Minimums of 500 events (beads) were collected for each cytokine/sample and median fluorescence intensities were obtained. Cytokine concentrations were calculated based on standard curve data using MasterPlex™ QT Analysis version 2 (MiraiBio, Alameda, Calif.). Results expressing picograms of cytokines per ml of serum two hours after administration are presented in FIG. 9.

While the free immune stimulant (IM) and Aldara induce very high levels of proinflammatory cytokines 2 hours after administration, immunotherapy compound AH-12 (which induced a cell mediated immune response at the site of administration in previous example) did not differ from untreated animals. This establishes the safety profile of the present immunotherapy compounds (peptide conjugates) relying on their ability to limit the in vivo dissemination of the TLR7/8 agonists.

In embodiments are provided immunotherapy compounds having the structure of Formula (I): DM-L-IM, wherein DM comprises a peptide from 17 to 45 amino acids in length comprising amino acid residues possessing helix forming properties wherein the DM is configured to form an amphipathic α-helix structure, and wherein the peptide is not derived from an antigen or immunogen and is a non-native sequence; wherein L is a linker; and IM is a toll-like receptor 7 (TLR7) and/or TLR8 agonist. In embodiments, these immunotherapy compounds are used for local administration wherein the compounds are insoluble in physiological conditions (e.g. pH and/or ion concentrations) forming depots or aggregates that are retained at the site of administration. The present immunotherapy compounds do not demonstrate induction of a systemic proinflammatory response, but they do advantageously induce a cell-mediated immune response.

2. kHL-12 and Derivatives Thereof

The study was designed to evaluate the respective antitumor activities and safety profile of conjugates and small molecules TLR7/8 agonists including (1) kHL-12, (2) ADJ-P3112(pHL-12) containing a lipid tail, (3) 3M-052, a TLR7/8 agonist conjugated to a lipid chain, (4) R848, a small molecule TLR7/8 agonist.

Six to eight-week-old female BALB/c mice (Charles River) were injected subcutaneously with 2×10⁵ CT26 cells on the right flank. When the tumors reached an average volume of ˜150 mm³, mice were randomized into seven groups of eight mice per group, approximately 10 days (13 days) post tumor cell grafting, referred as day 0. After randomization, tumor size and body weight were assessed three times a week for the duration of the study. Animals were sacrificed according to the following humane endpoints: tumor volume ≥2000 mm³, presence of necrotic or ulcerated tumor, impaired mobility including transient prostration or hunched posture, or interference with a vital physiological function. Treatments were initiated on day 1. All animals from group 1 to group 6 received three intratumoral injections of either formulated or non-formulated small molecule TLR7/8 agonists, or vehicle control, once every two days, on day 1, day 3 and day 5, in 50 μl delivery dose. Mice of group 7 were untreated. Animals from group 1 to group 4 were respectively treated with 100 nmol/dose/animal of kHL-12 or pHL-12 both prepared in 28 mM L-Histidine/Mannitol dilution buffer, or 3M-052 (MedChemExpress, teltralimod Cat. No.: HY-109104) prepared in ethanol/sesame oil (1:10), or R848 (Invivogen, R848 VacciGrade™, Cat. Code vac-r848) reconstituted in 28 mM L-Histidine/Mannitol dilution buffer. Mice from group 5 and group 6 received control vehicles in the delivery dose of 50 μl, 28 mM L-Histidine/Mannitol dilution buffer and ethanol/sesame oil (1:10), respectively.

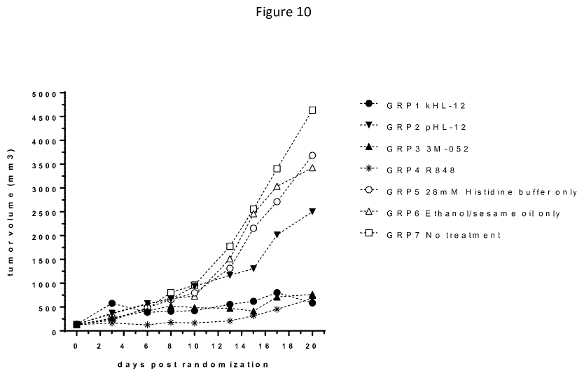

Median tumor volume across the seven groups are presented in FIG. 10. Changes in body weight across the seven groups are presented in FIG. 11. Body weight was normalized by subtracting the weight of the tumor to the overall body weight.

Results show that intratumoral kHL-12 (group 1) induces a stabilization of the tumor volume over time, an antitumoral activity that highly contrasts with the different control groups 5, 6 and 7 (FIG. 10). Moreover, the antitumoral activity of kHL-12 (group 1) is found comparable to groups that have received intratumoral administration of micellar formulation of 3M-052 (group 3) or of free agonist, R848/resiquimod, an imidazoquinoline and agonist of Toll-like receptors (TLRs) 7 and 8, (group 4). Compared to kHL-12, pHL-12 achieves a lower level of antitumor activity. FIG. 11 shows that the intratumoral administration of kHL-12 has limited impact on the body weight over time compared to the different control groups, reflecting its good safety profile. This result contracts with the negative impact of R848 and 3M-052 that promote a pronounced loss in body weight over time. The improved safety profile of kHL-12 compared to the other TLR7/8 agonists evaluated in the study relates to the depot forming property of the compound that prevent its systemic diffusion from the site of administration and consequently limiting the off-target side effects which lead to the induction of a deleterious systemic burst of pro-inflammatory cytokines.

### Example 7: Synthesis of Toll-Like Receptor 7 (TLR7) and/or TL-8 Agonist (Immunostimulant Moiety (IM))

The synthetic routes presented in the following Methods may be employed to prepare synthetic small molecule TLR7/8 agonist structures for certain embodiments of IM. More specifically, Method A can be used for IM in formulas (Ia), (Ib), (Ic), (Id), (Ih), (Ii), (Ij) and (Ik), while Methods B, C, D or E in conjunction with Method F can be employed for IM in formula (Il).

Method A: Synthesis of Formula (Ia) to (Id) and (Ih) to (Ik);

Scheme 1 presents the preparation of multiple members of the imidazoquinoline class of TLR7/8 agonists (Shukla, N.M.; Mutz, C.A.; Ukani, R.; Warshakoon, H. J.; Moore, D. S.; David, S. A. Bioorg. Med. Chem. Lett. 2010, 20, 6384-6386). See FIG. 14.

Step A-1.

Starting from a solution of 2,4-dichloro-3-nitroquinoline (1, 1.0 eq) in anhydrous dichloromethane (DCM), triethylamine (Et₃N, 1.3 eq) and the mono-protected diamine (2, 1.1 eq) are each added sequentially and the resulting mixture is refluxed at 45° C. After 0.5 h, the reaction is allowed to cool to room temperature (r.t.), then evaporated in vacuo. The resulting crude product can be isolated using flash chromatography to produce purified compound 3.

Step A-2.

To a solution of intermediate 3 in ethyl acetate (EtOAc) is added catalytic amounts of 10% platinum on carbon (10% Pt/C) and sodium sulfate (Na₂SO₄). The heterogeneous mixture is placed under hydrogen pressure (50-60 psi) for 4-6 h. The reaction is filtered through Celite, the filtered material is then washed with EtOAc (2×), and the combined filtrates evaporated in vacuo to obtain crude 4, typically of sufficient purity to be used for the next transformation.

Step A-3.

Triethylamine (1.5 eq) and acid chloride (5, 1.2 eq) are added to a solution of 4 (1.0 eq) in anhydrous tetrahydrofuran (THF), then the reaction mixture is stirred at r.t. for 4-8 h. The solvent is then removed in vacuo, and the crude residue taken up in EtOAc and washed with water (2×) and saturated aqueous sodium bicarbonate (NaHCO₃). The organic layer is then dried over anhydrous Na₂SO₄ and evaporated in vacuo to obtain crude 6, which can be purified by flash chromatography, but may be of sufficient quality to proceed directly to the final step.

Step A-4.

Compound 6 is dissolved in a minimum amount of methanol (MeOH), treated with an excess of 2M ammonia (NH₃) in MeOH, then transferred into a pressure vessel (e.g. Parr). The sealed vessel is heated to 145-150° C. overnight (18-24 h). The solvent is then removed in vacuo and the residue purified by flash chromatography or crystallization to yield the desired structure 7.

Utilizing Method A, the indicated IM can be synthesized from the specific diamines (2) and acid chlorides (5) shown in Table 5:

TABLE 5
Synthesis of IM via Method A

| Diamine (2) | Acid Chloride (5) | IM |
|---|---|---|
| 4-(Boc-aminomethyl)- | pentanoyl chloride (5a) | Formula |
| benzylamine (2c) | | (Ia) |
| 4-(Boc-aminomethyl)- | 2-ethoxyacetyl chloride (5b) | Formula |
| benzylamine (2c) | | (Ib) |
| 1-Boc-1,3-propanediamine (2a) | pentanoyl chloride (5a) | Formula |
| | | (Ic) |
| 1-Boc-1,3-propanediamine (2a) | 2-ethoxyacetyl chloride (5b) | Formula |
| | | (Id) |
| 1-Boc-1,3-propanediamine (2a) | 3-methoxypropanoyl chloride | Formula |
| | (5c) | (Ih) |
| 1-Boc-1,4-butanediamine (2b) | 3-methoxypropanoyl chloride | Formula |
| | (5c) | (Ii) |
| 1-Boc-1,4-butanediamine (2b) | pentanoyl chloride (5a) | Formula |
| | | (Ij) |
| 4-(Boc-aminomethyl)- | 3-methoxypropanoyl chloride | Formula |
| benzylamine (2c) | (5c) | (Ik) |

Method B; Synthesis of Precursor (15a) for Formula (II)

Scheme 2 outlines the synthesis of selected members of the imidazoquinoline class of TLR7/8 agonists (Shi, C.; Xiong, Z.; Chittepu, P.; Aldrich, C. C.; Ohlfest, J. R.; Ferguson, D. M. ACS Med. Chem. Lett. 2012, 3, 501-504; Schiaffo, C. E.; Shi, C.; Xiong, Z.; Olin, M.; Ohlfest, J. R.; Aldrich, C. C.; Ferguson, D. M. J. Med. Chem. 2014, 57, 339-347). See FIG. 5.

Step B-1.

A suspension of aminomalononitrile p-toluenesulfonate (8, 1.0 eq) in THF is treated with Et₃N (1.2 eq) at r.t. After stirring for 0.5 h, the solution becomes homogeneous and the orthoformate (10, 1.2 eq) is added. The mixture is then heated to reflux for 3 h. If the reaction is not complete at that time (TLC), the reaction is removed from heat, additional orthoformate (0.6 eq) introduced and the solution heated at reflux for an additional 2 h. When completed, the mixture is allowed to cool to r.t. to provide a solution containing the intermediate imidate. To this is sequentially added Et₃N (1.2 eq) and 1-amino-2-methylpropan-2-ol (9, 1.2 eq, can be prepared as described in Step B-5) and the reaction is stirred at r.t. overnight. The mixture is then concentrated in vacuo and the residue dissolved in DCM, which is then washed with saturated aqueous sodium carbonate (Na₂CO₃). The aqueous layer is extracted with DCM (3×). Next, the combined organics are washed with saturated aqueous brine (NaCl), dried over anhydrous magnesium sulfate (MgSO₄), filtered, and the filtrate evaporated in vacuo. Purification of the resulting crude material by flash chromatography affords 11.

Step B-2.

A solution of 11 (1.0 eq) in diiodomethane is heated to 80° C., then isoamylnitrite (4.0 eq) in chloroform (CHCl₃) is added over a period of 0.25-0.5 h. After the addition is complete, heating is maintained for 0.5 h, then the reaction is allowed to cool to r.t. and the solvent concentrated in vacuo. The crude product is purified by flash chromatography to yield 12.

Step B-3.

The catalyst is prepared from palladium acetate (Pd(OAc)₂, 0.05 eq) and triphenylphosphine (PPh₃, 0.1 eq), which are placed together in a dry flask and purged with argon for 0.25 h, then 1,2-dimethyoxyethane (DME) is added. The resulting suspension is stirred at r.t. for 5-10 min, then 12 (1.0 eq), 13 (1.5 eq) and 1.5 M Na₂CO₃ (aq) (3.0 eq) added sequentially. The reaction is heated at 100° C. for 3 h, then cooled to r.t. and the mixture is diluted with EtOAc and H₂O. The aqueous layer is extracted with EtOAc (3×). The combined organic layers are washed with saturated NaCl (aq), dried over anhydrous MgSO₄, filtered, and the filtrate is concentrated in vacuo. The resulting residue is purified by flash chromatography to give 14.

Step B-4.

A solution of 4 N HCl in dioxane (16 eq) is added to 14 (1.0 eq), then heated at reflux for 5 h. The reaction is cooled to r.t. and concentrated in vacuo. The residue is taken up in 10% MeOH in EtOAc and washed with saturated NaHCO₃(aq). The aqueous layer is extracted with 10% MeOH in EtOAc (3×). The combined organic layers are washed with saturated NaCl (aq), dried over anhydrous MgSO₄, filtered, and the filtrate is evaporated in vacuo. The crude residue is then purified by flash chromatography to afford the desired imidazoquinoline 15.

Step B-5. Synthesis of 1-amino-2-methylpropan-2-ol (9).

The title compound is prepared using a variation on the literature method (Close, W. J. J. Am. Chem. Soc. 1951, 73, 95-98). Isobutylene oxide (2,2-dimethyloxirane, 16, 1.0 eq) is combined with ammonium hydroxide (NH₄OH, 2 mL/mmol) and MeOH (1 mL/mmol). The mixture is stirred at r.t. for 12 h, and then slowly heated to 60° C. and stirred at that temperature for 2-3 h. The solvent is removed in vacuo, and the residue is distilled under atmospheric pressure to provide the desired product 9 in low yield.

As an example using the above Method B synthesis, compound 15a, an intermediate for the TLR7/8 agonists of formula (Il) can be synthesized in 25-30% yield from 1,1,1-triethoxypentane (triethyl orthovalerate, 10a, R₂═CH₂CH₂CH₂CH₃) and 2-aminophenylboronic acid (13a, R₃, R₄═H). Analogous compounds can be made with different R₂, R₃ and R₄ groups using Method B as well. In varying embodiments, a number of substituted 2-aminophenylboronic acids (13, (R₃=H, Me, Et, iPr, tBu, cyclopropyl, CF₃, F, Cl, Br, NO₂, OPG₁, OMe, OCF₃, NHPG₁, NMePG₂, NEtPG₂, NHCOMe, CN, CO₂PG₃, CO₂Me, CO₂Et, CO₂iPr, CONHMe, CONHEt, SO₂Me; R₄=H, Me, CF₃, F, Cl, NO₂, OPG₂, OMe, OCF₃, CN, CO₂PG₃; PG₁=H, tBu, CH₂Ph, TBDMS, COMe; PG₂=H, Alloc, Boc, Cbz, Fmoc; PG₃=H, tBu, CH₂Ph)) are available commercially, while orthoesters (10, R₂═CH₃, CH₂CH₃, CH₂CH₂CH₃, CH₂CH₂CH₂CH₃, CH₂OCH₂CH₃, CH₂CH₂OCH₃) can be made relatively easily using the Pinner reaction (McElvain, S. M.; Nelson, W. J. Am. Chem. Soc. 1942, 64, 1825-1827; Roger, R.; Neilson, D. G. Chem. Rev. 1961, 61, 179-211; Noe, M.; Perosa, A.; Selva, M. Green Chem. 2013, 15, 2252-2260) from nitriles and alcohols under acidic conditions or in the presence of a Lewis acid, such as BF₃-etherate (Corey, E. J.; Raju, N. Tetrahedron Lett. 1983, 24, 5571-5574), with a limited selection also obtainable from organic chemical reagent vendors. Such aryl boronic acids can also be prepared from organometallic reagents (i.e. Grignards, organolithiums) and trialkyl borates (“Synthesis of Organoboronic Acids, Organoboronates, and Related Compounds” Chapter 6, in Practical Functional Group Synthesis, Stockland, R. A., Jr., John Wiley & Sons, Inc., Hoboken, N.J., 2016, pp 515-555). Lastly, imidazoquinolines with different N1-substituents can also be made through the process outlined in Method B through the use of different amino alcohols or diamines (Lason, et al. ACS Med. Chem. Lett. 2017, 8, 1148-1152), such as 2a, 2b and 2c. Hence, Method B also has applicability to the IM in Formula (Ia), (Ib), (Ic), (Id), (Ih), (Ii), (Ij) and (Ik).

Another procedure that can be employed to access compound 15 and analogues is described in Method C, which is based on syntheses reported in WO 2013/067597 (PCT/AU2012/001387).

Method C; Synthesis of Precursor Compounds (15)

Scheme 3 outlines the synthesis of selected members of the imidazoquinoline class of TLR7/8 agonists. See FIG. 16.

Step C-1.

A mixture of 2,4-dihydroxyquinoline (17) in concentrated nitric acid (HNO₃, 0.25 mL/mmol) and glacial acetic acid (HOAc, 1 mL/mmol) is stirred at 105° C. for 1 h, then cooled to r.t. The reaction is quenched by the addition of H₂O, upon which a yellow solid precipitate is formed. The solid is filtered, washed with cold H₂O, and then dried to provide 18.

Step C-2.

To a solution of 18 (1.0 eq) in phosphorous oxychloride (POCl₃, 1.65 mL/mmol) is added Et₃N (1.0 eq), then the reaction mixture is heated to 120° C. and stirred for 3 h. After cooling, the solvent is removed in vacuo. The residue is poured into ice-water and extracted with DCM (2-3×). The combined organic phase is washed sequentially with saturated NaHCO₃(aq) and brine, dried over anhydrous Na₂SO₄, filtered and the filtrate is concentrated in vacuo to give the desired product 19, which could be used directly in the next step.

Step C-3.

To a solution of 19 (1.2 eq) and Et₃N (1.5 eq) in DCM is added 1-amino-2-methylpropan-2-ol (9, 1.0 eq, from Step B-5) dropwise. The mixture is stirred at reflux for 12 h. The solution is cooled to r.t., then washed with brine, dried over anhydrous Na₂SO₄, filtered, and the filtrate concentrated in vacuo to obtain crude product 20, which is purified by flash chromatography.

Step C-4.

A mixture of 20 (1.0 eq) and 10% Pt/C (40 mg/mmol) in EtOAc is placed under a hydrogen atmosphere (50-55 psi) at r.t. for 4 h. The mixture is then filtered through Celite, the solid washed with EtOAc, and the combined filtrate concentrated in vacuo. The resulting crude 21 is purified by flash chromatography or used as is in the next step.

Step C-5.

To a solution of 21 (1.0 eq) and Et₃N (2.0 eq) in DCM is added the acid chloride (5, 1.2 eq (e.g., 5a though 5k)). The reaction is stirred for 3 h, then washed with saturated brine. The organic layer is concentrated in vacuo and the residue purified by flash chromatography to provide the desired product 22.

Step C-6.

A mixture of compound 22 (1.0 eq) in excess NH₃-MeOH is placed in a pressure vessel, sealed and stirred at 160° C. for 8 h. The solvent is then removed in vacuo, and the resulting crude residue is purified by flash chromatography (requires Et₃N in the elution solvent) to yield 15.

The process of Method C in Scheme 3 (FIG. 16) can be employed with acid chlorides 5a, 5b, 5c, 5e, 5f, 5g, 5h, 5i and 5j to give the imidazoquinolines 15a, 15b, 15c, 15e, 15f, 15g, 15h, 15i and 15j, respectively. In addition, use of acid chloride 5k provides imidazoquinoline 15k possessing an N-Cbz protecting group, which can be removed employing the same procedure described in Step E-12 to yield 15d.

Method D; Alternative Synthesis Route for Preparation of Precursor 15a, 15b, 15c, 15d, 1e, 15f, 15g, 15h, 15i and 15j Compounds

In a variation of the route described above in Method C, an alternative is provided in Scheme 4 (FIG. 17) with many similar transformations, although arranged in a different order. Most notably, the end stage installation of the 4-amino group relies on classical rearrangement chemistry (Gerster, J. F.; Lindstrom, K. J.; Miller, R. L.; et al. J. Med. Chem. 2005, 48, 3481-3491). As depicted in Scheme 4, the process begins with the nitration of 4-hydroxyquinoline (23) using the method of Step C-1. The resulting 3-nitro-4-hydroxy quinoline (24) is then subjected to a sequence involving chlorination (Step C-2) to provide 25, reduction of the nitro group with hydrogen gas over Raney nickel in ethanol (e.g., Step C-4) to give 26, and finally acylation (Step C-5) to yield 27. Next, high temperature, concentrated reaction conditions will lead to an acid-induced, dehydrative ring closure of 27 experienced essentially simultaneously with displacement of the chloride by the amino group of 1-amino-2-methylpropan-2-ol (9) to produce 28. See FIG. 17.

Step D-1.

To a solution of 28 (1.0 eq) in DCM is added meta-chloroperoxy-benzoic acid (70% active oxygen, 1.2 eq) at 0° C. The reaction is maintained at 0° C. for 0.5 h, warmed to r.t. and stirred for 2 h. The mixture is then concentrated in vacuo. The resulting solid residue is dissolved in H₂O and made slightly basic with dilute NaOH (aq), which leads to precipitation of the N-oxide. The solid is collected by vacuum filtration, washed with H₂O, and air-dried to give still impure product. The collected solid is suspended in toluene, heated to reflux with stirring in order to azeotropically remove the H₂O from the product. Once no further H₂O is obtained, the solid is again collected by vacuum filtration, washed with toluene, and is dried to provide the slightly colored product 29.

Step D-2.

A solution of 29 (1.0 eq) in DCM is first treated with concentrated ammonium hydroxide (NH₄OH, NH₃ (aq)), followed by the dropwise addition of p-toluenesulfonyl chloride (Tos-Cl, 1.0 eq) in DCM with vigorous agitation at 0° C. over 0.25 h. A clear exothermic reaction is observed, and a solid precipitate formed during addition. Upon completion of the addition, the mixture is maintained at 0° C. for 0.5 h, then warmed to r.t. and stirred for 2 h. The precipitate is collected by vacuum filtration, washed with DCM and H₂O, then is pressed partially dry. The still moist solid is slurried with MeOH, collected by vacuum filtration, and dried. The solid is treated again with MeOH and refluxed for 5 min, then again collected and dried as before. Crystallization may be required to obtain pure product 15.

The process of Method D in Scheme 4 (FIG. 17) can be employed with acid chlorides 5a, 5b, 5c, 5e, 5f, 5g, 5h, 5i and 5j to give the imidazoquinolines 15a, 15b, 15c, 15e, 15f, 15g, 15h, 15i and 15j, respectively. Similar to as described in Method C, the procedure of Step E-12 can be employed to convert 15k (made from 5k) into 15d.

Method E; Alternative Synthesis Route for Preparation of Precursor 15a, 15b, 15c, 15d, 1e, 15f, 15g, 15h, 15i and 15j Compounds

In a variation of the route described above in Method D and analogous to a reported strategy (Bioorg. Med. Chem. Lett. 2009, 19, 2211-2214), another alternative for precursor molecules is provided in Scheme 5 proceeding through two of the same intermediates as in Method D. See FIG. 18.

Step E-1.

Using a route adapted from the literature method for N1-unsubstituted adenine derivatives (J. Med. Chem. 2006, 49, 3354-3361), 2-nitroacetaldehyde oxime is prepared in situ by adding nitromethane (1.1 eq) dropwise to a solution of NaOH (3.0 eq) in water at 0° C. The mixture is then warmed to 40° C. and nitromethane (1.1 eq) is again introduced slowly at that temperature, which is maintained until the solution becomes clear. The reaction mixture is then heated to 50-55° C. for 2-5 min, cooled to near rt, and poured onto ice. This solution is acidified with HCl (conc.), then immediately added to a filtered solution of anthranilic acid (30, 1.0 eq) in 0.5N HCl in water. The reaction mixture, from which a precipitate forms, is maintained at r.t. for 12 h. The solid is collected by filtration, washed with water, and dried at 100-110° C. to yield 31 as a yellow powder.

Step E-2.

A heterogeneous mixture of 31 (1.0 eq) in acetic anhydride is heated to 100-105° C. until a clear solution is obtained. Heating is removed and potassium acetate (1.03 eq) added. The reaction is then heated to reflux for 0.25 h with vigorous stirring, until a solid begins to form. The mixture is allowed to slowly cool to r.t., then the precipitate collected by filtration and washed with glacial acetic acid until no further color is seen in the wash. The solid is suspended in water, filtered, washed with water and dried at 100-110° C. to obtain 3-nitroquinolin-4-ol (24).

Step E-3.

Similar to Step C-2, 24 (1.0 eq) is carefully added to phosphorous oxychloride (13-15 eq) with stirring. The reaction mixture is heated to reflux for 0.5 h. The volatiles are then evaporated in vacuo and the liquid residue poured into crushed ice with stirring. After 1 h, the solid that is formed is collected by filtration and washed with cold H₂O. This is then dissolved in DCM containing a minimum amount of MeOH, washed with ice cold 1 N NaOH (aq), and the organic layer dried over Na₂SO₄ (anhydrous) and activated charcoal. The solution is filtered through Celite, washed with DCM and the combined filtrate evaporated in vacuo. Trituration of the residue with diethyl ether gives 4-chloro-3-nitroquinoline (25) after drying under vacuum.

Step E-4.

To a solution of 25 (1.0 eq) and N,N-diisopropylethylamine (DIPEA) (2.5 eq) in toluene and isopropanol (iPrOH) (4:1) is added 9 (2.0 eq) and the mixture heated to 70° C. for 0.5 h at which time a precipitate forms. The reaction is cooled and the solid collected by filtration, which is then washed sequentially with toluene:iPrOH (7:3), diethyl ether and cold H₂O. The residue is dried at 80° C. to obtain 32 of sufficient purity to be used in the next reaction.

Step E-5.

32 (1.0 eq) is dissolved in MeOH and hydrogenated using 10% Pd/C as catalyst under a H₂ pressure of 50-60 psi for 4 h. The mixture is then filtered using Celite to remove the catalyst and the filtrate evaporated in vacuo to leave 33, which is purified by crystallization or flash chromatography if necessary.

Step E-6.

33 (1.15 eq), 34 (1.0 eq), 0-(7-azabenzotriazol-1- yl)-N,N,N′,N′-tetramethyl uronium hexafluorophosphate (HATU) (1.4 eq), Et₃N (3.5 eq) and 4-dimethylaminopyridine (DMAP) (cat.) are dissolved in dimethylformamide (DMF) and the reaction stirred for 10-12 h. The solvent is evaporated in vacuo and the residue dissolved in EtOAc. The organic is washed with H₂O, then dried over Na₂SO₄ (anhydrous), filtered and the filtrate concentrated in vacuo. The crude residue thus obtained is dissolved in EtOH and aqueous NaOH (4.0 eq) is added. The mixture is heated to reflux for 5-6 h, then the solvent removed in vacuo. The crude product is purified using flash chromatography to provide 35. The use of the acid rather than the acid chloride permits the use of Boc protection (34l) in addition to Cbz (34k) for accessing compound 15d.

Step E-7.

To a solution of 35 (1.0 eq) in DCM:CHCl₃ (1:1) and MeOH (10% by volume) is added meta-chloroperoxybenzoic acid (2.5 eq) and the reaction heated to reflux for 30 min. The mixture is concentrated in vacuo and the residue purified using flash chromatography to give the N-oxide 36.

Step E-8.

35 (1.0 eq) is dissolved in anhydrous DCM and benzoyl isocyanate (1.5 eq) added, then the mixture heated to reflux for 30 min. The reaction is concentrated in vacuo and the resulting residue dissolved in anhydrous MeOH. Excess NaOMe is added and the reaction again refluxed for 2-3 h. After evaporation of solvent in vacuo, the crude residue is purified using flash chromatography to obtain 15.

The process of Method E in Scheme 5 (FIG. 18) can be employed with acids 34a, 34b, 34c, 34e, 34f, 34g, 34h, 34i and 34j to give the imidazoquinolines 15a, 15b, 15c, 15e, 15f, 15g, 15h, 15i and 15j, respectively. Imidazoquinolines 15k and 15l, accessed from acids 34k and 34l, respectively, are deprotected as described below using the procedures in Steps E-11 and E-12, respectively, to yield 15d.

Step E-9. Synthesis of N-Ethyl Glycine Methyl Ester (38).

A solution of methyl glycinate 37 (3.0 eq) in anhydrous methanol is treated with acetaldehyde (1.0 eq), sodium cyanoborohydride (NaCNBH₃, 1.0 eq) and 5-6 drops of acetic acid. The resulting reaction is stirred for 12 h. At that time, HCl (conc.) is carefully added to the mixture until the pH reaches 1-2 (pH paper). The solvent is then removed in vacuo and the crude residue purified using flash chromatography to afford 38.

Step E-10. Synthesis of Cbz-Protected N-Ethyl Glycine (34k)

To a solution of 38 (1.0 eq) in THF is added 2N NaOH (3.0 eq) and benzyl chloroformate (1.6 eq). After stirring 1 h at rt, the pH is adjusted to 1-2 and the layers separated. The aqueous phase is extracted with EtOAc (2×). The combined organic layers are dried (anhydrous MgSO₄), filtered, and concentrated in vacuo to leave a crude residue, which is then purified by crystallization or flash chromatography to provide 34k.

Step E-11. Synthesis of Boc-Protected N-Ethyl Glycine (34l).

To 38 (1.0 eq) in MeOH is added di-tert-butyl dicarbonate (Boc₂O, 2.0 eq) and Et₃N (1.2 eq) and the reaction stirred at r.t. until TLC indicated complete reaction (approx. 2 h). The solvent is evaporated in vacuo, then the residue dissolved in THF:MeOH (3:1). An aqueous solution of lithium hydroxide (LiOH, 4.0 eq) is added to the mixture, which is then stirred at r.t. for 12 h. The volatiles are removed in vacuo and H₂O added to the residue. The solution is acidified to pH 2 or lower using 10% HCl, then EtOAc added and the layers separated. The aqueous layer is extracted with EtOAc (lx), then the combined organic layers are dried (anhydrous MgSO₄), filtered, and concentrated in vacuo to afford crude 34l, which is purified by crystallization or flash chromatography.

Step E-12. Deprotection of Cbz Protecting Group.

A solution of 15k (1.0 eq) in 95% EtOH (0.1 M) with 10% Pd/C (0.1 eq) catalyst is stirred under an atmosphere of H₂ for 72 h, after which time the mixture was filtered through a Celite pad. The pad was washed with EtOAc and the combined filtrate and washings are concentrated in vacuo to provide 15d, which is purified by crystallization or flash chromatography.

Step E-13. Deprotection of Boc Protecting Group.

15l (1.0 eq) is dissolved in 3 mL of TFA and stirred at r.t. for 0.5 h. The solvent is removed in vacuo to yield 15d as its trifluoroacetate salt, which can be neutralized and purified by flash chromatography to obtain the pure free base.

Method F; Synthesis of IM Formula (II)

The structure required for formula (Il) can be accessed from 15 using the synthetic route shown in Scheme 6 and described below as Method F. All transformations are known to one skilled in the art. See FIG. 19.

For this route, the 4-amino group of 15 (R₂═CH₃, CH₂CH₃, CH₂CH₂CH₃, CH₂CH₂CH₂CH₃, CH₂OCH₂CH₃, CH₂CH₂OCH₃,CH₂CH(CH₃)₂, CH₂CH₂CH₂CH₂CH₃, CH₂Ph, CH₂NYCH₂CH₃ (Y=H, Cbz, Boc)) must first be protected so as to not interfere in subsequent chemistry. A standard method for its installation (Boc₂O, Et₃N, as in Step E-11) is employed. The alcohol in the resulting Boc-protected product 39 is then activated as its p-nitrophenyl carbonate by treatment with p-nitrophenyl chloroformate in the presence of base, with two alternative reactions for this transformation shown. Other activated moieties can also be used here, such as pentafluorophenyl (OPfp) or succinimide (OSu). Compound 40 can then be reacted with N-mono-protected diamines (41a, B=—NH—) or amino alcohols (41b, B=—O—) to produce the urethanes (42a, B=—NH—) or carbonates (42b, B=—O—), respectively. The protecting group (PG) on 41 is preferably orthogonal to the Boc (i.e. Cbz, Fmoc, Alloc), but could also even be Boc, since at this stage, the 4-amino group does not require protection as its free state is not expected to interfere with subsequent transformations, so its removal should not be detrimental. However, the N-protection on the C₂-substituent of 15, when present, must remain in place so that this secondary amine does not interfere with chemistry utilized in the formation of the conjugates. Deprotection of PG in 42 then provides the structure required for Formula (I). See FIG. 19.

### Example 8: Intratumoral Treatment

In this example, HL-6X2 or HL-6X3 (See FIG. 13), designed for intracellular release of TLR7/8 agonist through the presence of a cathepsin B cleavable linker (cathepsin-B being present in the endosome of antigen presenting cells) is injected into a tumor (intratumoral administration), in immunocompetent BALB/c mice comprising syngeneic (e.g. allograft) tumors from CT-26 (colon carcinoma) tumor cells. One-hundred (100) female BALB/c mice (Charles River) are injected subcutaneously with 2×10⁵ CT26 cells on the right flank. When the tumor size reaches an average volume around 100 mm³, mice are randomized into five (5) groups of ten (10) mice per group and dosed with the test articles (HL-6X2) or the vehicle control. Mice receive one (1) intratumoral injection of either a dosing range of each of HL-6X2 and HL-6X3 (e.g., 11 nmol, 33 nmol, 100 nmol) in 28 mM L-Histidine/Mannitol solution, or a dose of kHL-12 (100 nmol) in 28 mM L-Histidine/Mannitol solution or 28 mM L-Histidine/Mannitol solution as a vehicle control, according to the schedule shown in Table 6. Tumor size is assessed daily, once tumors are palpable (e.g., day 3 tumor cell post-dosing). Tumor measurements begin daily up to the randomization day, and then tumor size and bodyweight are assessed three times per week for the duration of the study. Animals are removed from study when tumor volumes reach a 2000 mm³ volume or according to animal care guidelines.

TABLE 6
Study Design

| | | | | | Intra- |
|---|---|---|---|---|---|
| | | | | | tumoral |
| | Num- | | CT26 tumor cells | Randomi- | adminis- |
| | ber of | | s.c injections | zation | tration |
| Group | Mice | Conditions | D 0 | D 7 | D 7 |
| 1 | 10 | HL-6X2 or | 2 × 10⁵ CT26 | ✓ | 50 μl/ |
| | | HL-6X3 | cells/100 | | dose/ |
| | | 100 nmol in | μl/animal | | animal |
| | | 50 μl | on the right | | |
| 2 | 10 | HL-6X2 or | flank | | 50 μl/ |
| | | HL-6X3 | | | dose/ |
| | | 33 nmol in | | | animal |
| | | 50 μl | | | |
| 3 | 10 | HL-6X2 or | | | 50 μl/ |
| | | HL-6X3 | | | dose/ |
| | | 11 nmol in | | | animal |
| | | 50 μl | | | |
| 4 | 10 | kHL-12 | | | 50 μl/ |
| | | 100 nmol in | | | dose/ |
| | | 50 μl | | | animal |
| 5 | 10 | Vehicle | | | 50 μl/ |
| | | solution | | | dose/ |
| | | (50 μl) | | | animal |

The study shows that HL-6X2, HL-6X3, and/or kHL-12 are capable of inhibiting and/or slowing the growth of cancer in vivo, as exemplified by the murine CT-26 model used herein.

### Example 9: Safety Profile of Oncology Therapy Compounds (Peptide Conjugates) Compared to Unmodified IM (TLR7 and/or TLR8 Agonist)

Eighty (80) female BALB/c mice (Charles River) are randomized according to the body weight into sixteen (16) groups of five (5) mice per group. Mice will receive on the right flank one (1) subcutaneous injection of either a dosing range of HL-6X2,HL-6X3,or free TLR7/8 agonist (formula (Ia)-NH₂(“FreeIM”)), or dose of kHL-12 or 28 mM L-Histidine/Mannitol dilution buffer as a vehicle control, according to the schedule Table 7. The clinical examination (skin reaction and swelling persistence) at the injection sites is recorded post injection and before each blood sample. Blood tests for serum collection and later pro-inflammatory cytokine quantitative analysis is performed 2 h and 24 h or 6 hand 48 h post-test articles and vehicle administration according to the schedule Table 7. Body weight is assessed daily for the duration of the study.

TABLE 7

| | | | Subcutaneous administration | | | | |
|---|---|---|---|---|---|---|---|
| | | | 50 ul/dose/animal in | | | | |
| | | | the right flank | Serum collection | | | |
| Group | Number | Conditions | T0 | T2 H | T6 H | T24 H | T48 H |
| 1 | 5 | HL-6X2 or HL-6X3 | X | X | | X | |
| | | 100 nmol in 50 μl | | | | | |
| 2 | 5 | HL-6X2 or HL-6X3 | X | | X | | X |
| | | 100 nmol in 50 μl | | | | | |
| 3 | 5 | HL-6X2 or HL-6X3 | X | X | | X | |
| | | 33 nmol in 50 μl | | | | | |
| 4 | 5 | HL-6X2 or HL-6X3 | X | | X | | X |
| | | 33 nmol in 50 μl | | | | | |
| 5 | 5 | HL-6X2 or HL-6X3 | X | X | | X | |
| | | 11 nmol in 50 μl | | | | | |
| 6 | 5 | HL-6X2 or HL-6X3 | X | | X | | X |
| | | 11 nmol in 50 μl | | | | | |
| 7 | 5 | kHL-12 | X | X | | X | |
| | | 100 nmol in 50 μl | | | | | |
| 8 | 5 | kHL12 | X | | X | | X |
| | | 100 nmol in 50 μl | | | | | |
| 9 | 5 | Free IM | X | X | | X | |
| | | 100 nmol in 50 μl | | | | | |
| 10 | 5 | Free IM | X | | X | | X |
| | | 100 nmol in 50 μl | | | | | |
| 11 | 5 | Free IM | X | X | | X | |
| | | 33 nmol in 50 μl | | | | | |
| 12 | 5 | Free IM | X | | X | | X |
| | | 33 nmol in 50 μl | | | | | |
| 13 | 5 | Free IM | X | X | | X | |
| | | 11 nmol in 50 μl | | | | | |
| 14 | 5 | Free IM | X | | X | | X |
| | | 11 nmol in 50 μl | | | | | |
| 15 | 5 | vehicle | X | X | | X | |
| 16 | 5 | Vehicle | X | | X | | X |

The study shows that HL-6X2, HL-6X3, or kHL12, is capable of reducing the induction of systemic serum cytokines compared to the free immunostimulant used herein.

While certain embodiments have been described in terms of the preferred embodiments, it is understood that variations and modifications will occur to those skilled in the art. Therefore, it is intended that the appended claims cover all such equivalent variations that come within the scope of the following claims.

1. An immunotherapy compound having the structure of Formula (I):
DM-L-IM,
wherein DM comprises a peptide from about 18 to about 45 amino acids in length comprising amino acid residues possessing helix forming properties wherein the DM is configured to form an amphipathic α-helix structure, and wherein the peptide sequence does not comprise a T cell epitope and/or a B cell epitope and is a non-natural sequence;
wherein L is a linker; and,
IM is a toll-like receptor 7 (TLR7) and/or TLR8 agonist selected from: 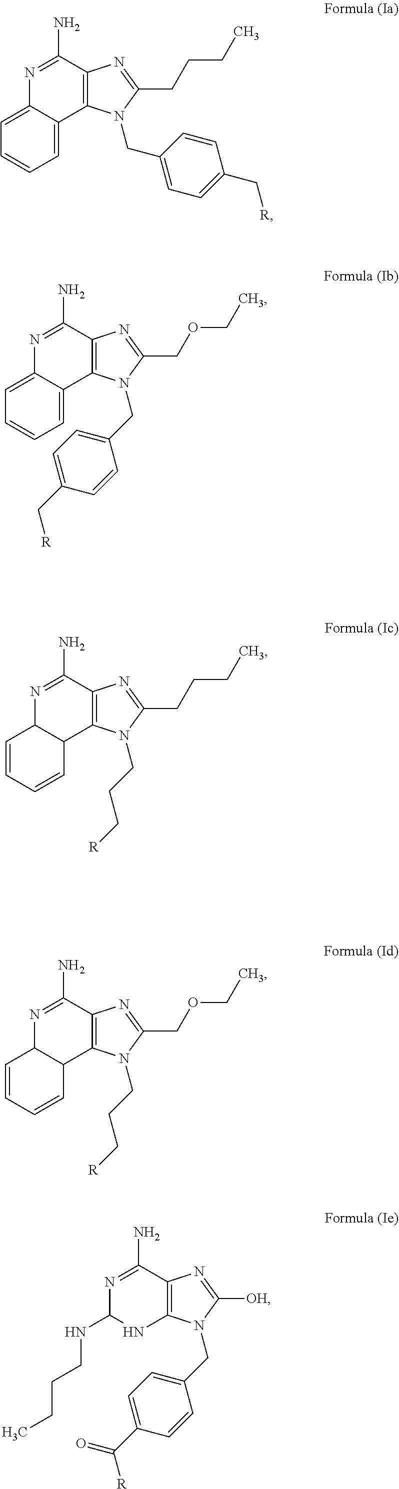 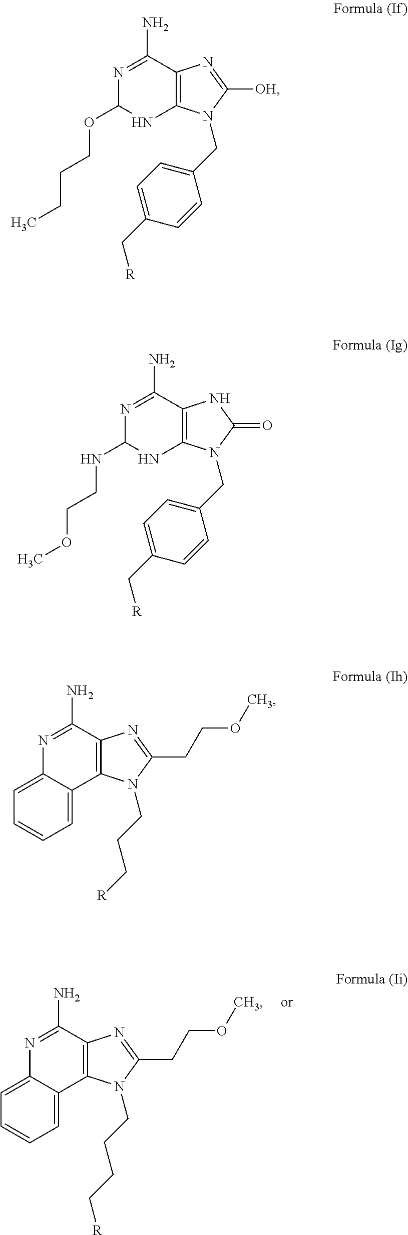 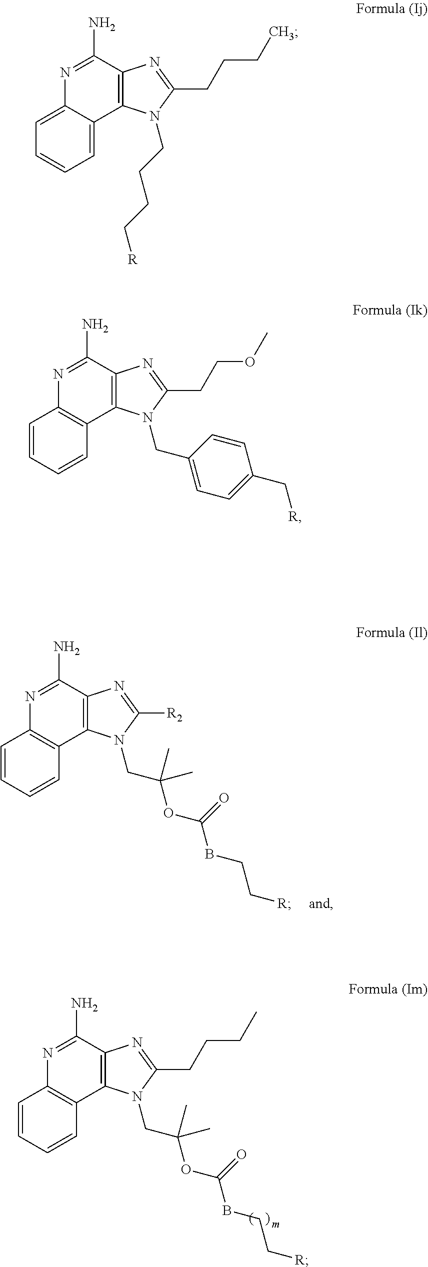
wherein:
R₂ is selected from:
CH₃, —CH₂CH₃, —CH₂CH₂CH₃, —CH₂CH(CH₃)₂, —CH₂CH₂CH₂CH₃, —CH₂CH₂CH₂CH₂CH₃, CH₂OCH₂CH₃, —CH₂CH₂OCH₃, —CH₂NHCH₂CH₃, and —CH₂Ph; and,
R comprises the linker connecting the IM to an amino group or carboxyl group of the peptide at the peptide termini or the lateral chain of an amino-acid such as lysine or glutamine, wherein the linker is -[A1]—NH—, and A1 is selected from:
A2-A3-(CH₂)ₓ—CO—,
A2-A3-CH₂—O—CH₂—CO—,
A2-A3-(CH₂)ₓ—O—(CH₂)ₓ—O—(CH₂)ₓ—O—(CH₂)ₓ—CO—,
A2-Valine-Alanine-A4-,
A2-Valine-Citrulline-A4-,
A2-Glutamate-Valine-Citrulline-A4-, or
A2-Phenylalanine-Lysine-A4-; wherein:
A2 is selected from:
A5-(CH₂)ₓ-A6-, -A5-(CH₂)ₓ—O—(CH₂)ₓ—O—(CH₂)ₓ—O—(CH₂)ₓ-A3-,
A5-(CH₂)₂—(O—CH₂—CH₂)ₓ-A6-
A5-CH₂—O—CH₂-A6-,
A5-(CH₂)ₓ-A6-,
A5-(CH₂)ₓ—O—(CH₂)ₓ—O—(CH₂)ₓ—O—(CH₂)ₓ-A6-, or,
A5-NH—(CH₂)₂—O—(CH₂)-A6-;
A3 is —CO— or —NH—;
A4 is p-aminobenzyloxy carbonyl (PABC): 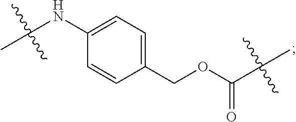 [SMILES: *Nc1ccc(COC(*)=O)cc1]
or nothing;
A5 and A6 are —CO— or —NH—, one or more natural or non-natural amino-acids, or nothing;
B is selected from O and NH;
m is any integer from 1 to 11; and,
x is any integer from 1 to 12, or wherein x is any integer from 2 to 12.

2. The compound of claim 1, wherein the IM is derived from or comprises a compound of Formula 15: 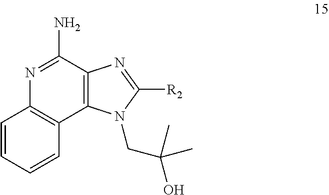
wherein R₂ is an alkyl optionally selected from the group consisting of CH₂CH₂CH₂CH₃, CH₂OCH₂CH₃, CH₂CH₂OCH₃, CH₂NHCH₂CH₃, CH₃, CH₂CH₃, CH₂CH₂CH₃, CH₂CH(CH₃)₂, CH₂CH₂CH₂CH₂CH₃, CH₂Ph, and CH₂NCbzCH₂CH₃.

3. The compound of claim 2, wherein IM is selected from the group consisting of:

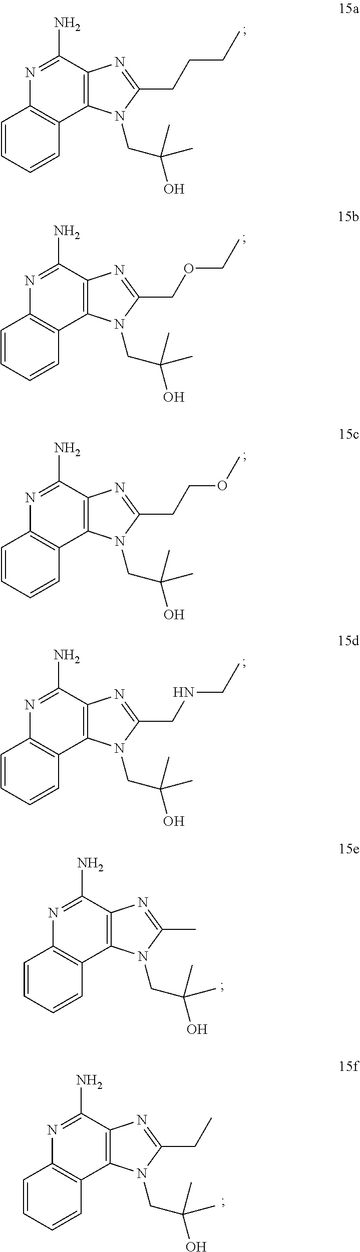 [SMILES: CCCCc1nc2c(N)nc3ccccc3c2n1CC(C)(C)O ; CCOCc1nc2c(N)nc3ccccc3c2n1CC(C)(C)O ; COCCc1nc2c(N)nc3ccccc3c2n1CC(C)(C)O ; CCNCc1nc2c(N)nc3ccccc3c2n1CC(C)(C)O ; Cc1nc2c(N)nc3ccccc3c2n1CC(C)(C)O ; CCc1nc2c(N)nc3ccccc3c2n1CC(C)(C)O]

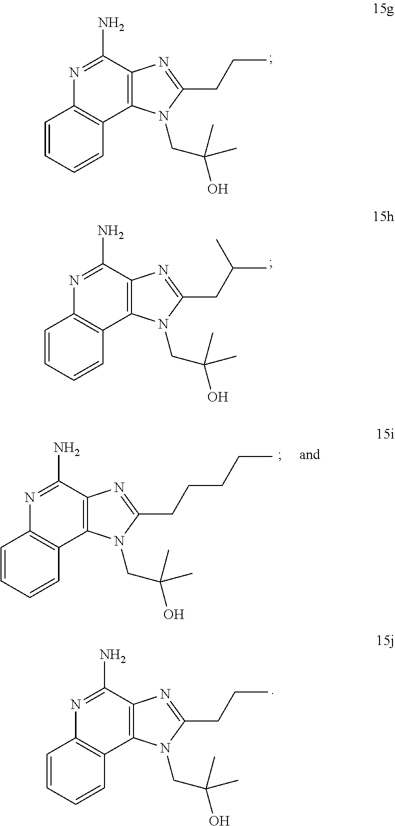 [SMILES: CCCc1nc2c(N)nc3ccccc3c2n1CC(C)(C)O ; CC(C)Cc1nc2c(N)nc3ccccc3c2n1CC(C)(C)O ; CCCCCc1nc2c(N)nc3ccccc3c2n1CC(C)(C)O ; CCCc1nc2c(N)nc3ccccc3c2n1CC(C)(C)O]

4. The compound of claim 1, wherein the DM further comprises a hydrophobic moiety covalently attached to a terminal amino acid of the peptide.

5. The compound of claim 4, wherein the hydrophobic moiety is selected from C₈F₁₇—(CH₂)₂—CO—, CH₃(CH₂)₁₂CO—, CH₃(CH₂)₁₄CO—, CH₃(CH₂)₁₆CO—, or

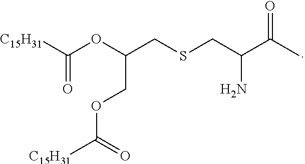

6. (canceled)

7. The compound of claim 1, wherein the peptide sequence comprises an amino acid sequence of RRLL(5)A(7)LAL(11)A(13)LLRRL (SEQ ID NO: 1) wherein amino acid positions (5), (7), (11) and (13) are each selected from A, L, or H.

8. The compound of claim 1, wherein the peptide comprises an amino acid sequence selected from RRLLHAHLALHAHLLRRLK (SEQ ID NO: 2), RRLLAAHLALHAALLRRLK (SEQ ID NO:3), or RRLLHALLALLAHLLRRLK (SEQ ID NO:4).

9. The compound of claim 1 selected from the group consisting of:
Ac-RRLLHAHLALHAHLLRRLK(ADJ12)-NH₂(named HH-12),
Ac-RRLLAAHLALHAALLRRLK(ADJ12)-NH₂(named AH-12),
Ac-RRLLHALLALLAHLLRRLK(ADJ12)-NH₂(named HL-12),
K(Ac)—RRLLHALLALLAHLLRRLK(ADJ12)-NH₂(named kHL-12),
K(Ac)—RRLLAAHLALHAALLRRLK(ADJ12)-NH₂(named kAH-12),
K(Pam)-RRLLHALLALLAHLLRRLK(ADJ12)-NH₂(named pHL-12), and
K(Pam)-RRLLAAHLALHAALLRRLK(ADJ12)-NH₂(named pAH-12),
wherein Pam is palmitoyl; Ac is acetyl; and, ADJ12 is derived from Formula I(a) where
R is —NH—CO—CH₂—O—CH₂—CO—NH—((CH₂)₂O)₃—(CH₂)₂—COOH, or is 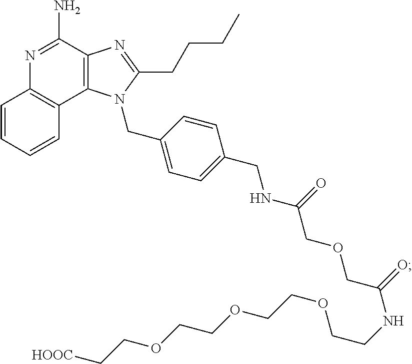 [SMILES: CCCCc1nc2c(N)nc3ccccc3c2n1Cc1ccc(CNC(=O)COCC(=O)NCCOCCOCCOCCC(=O)O)cc1]
or is selected from the group consisting of: 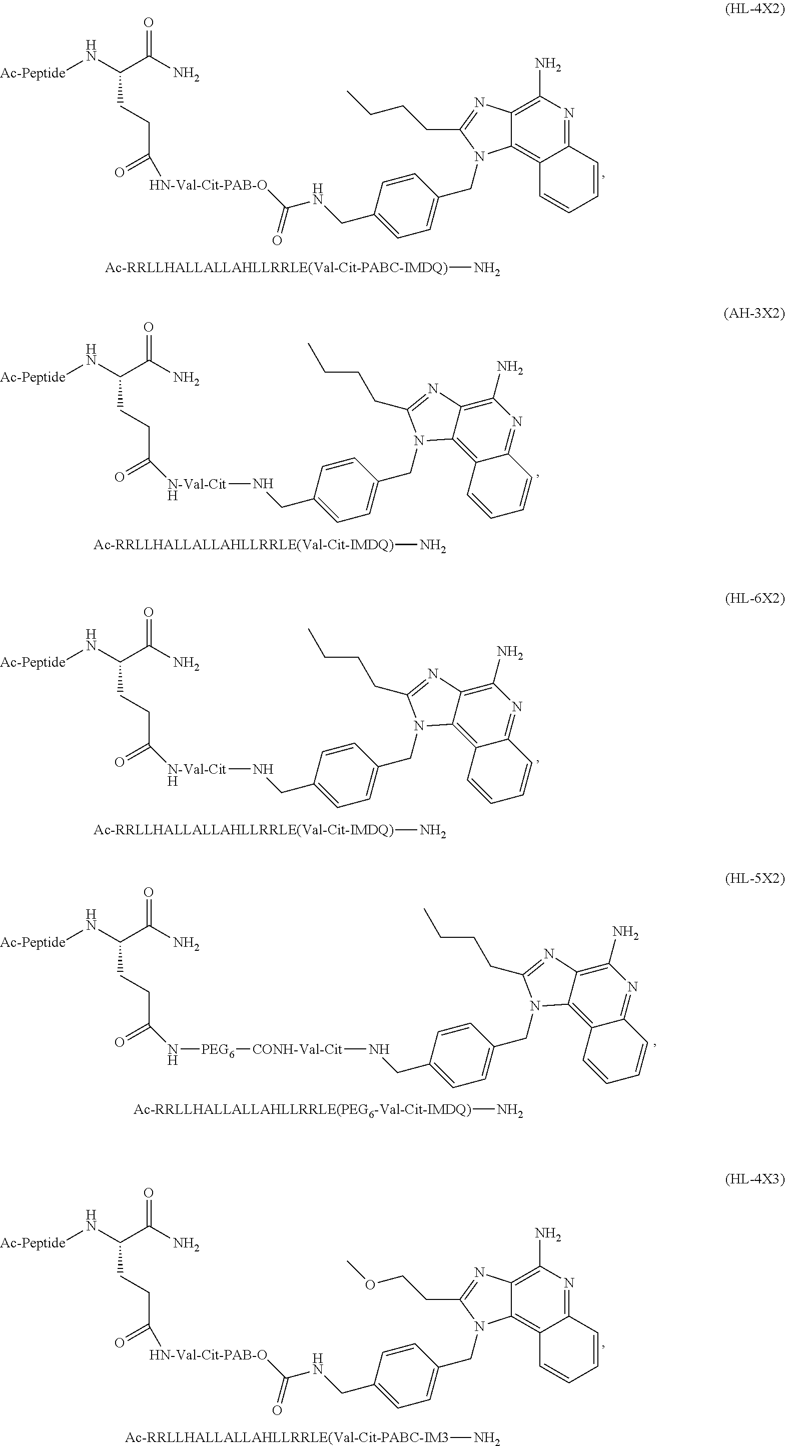 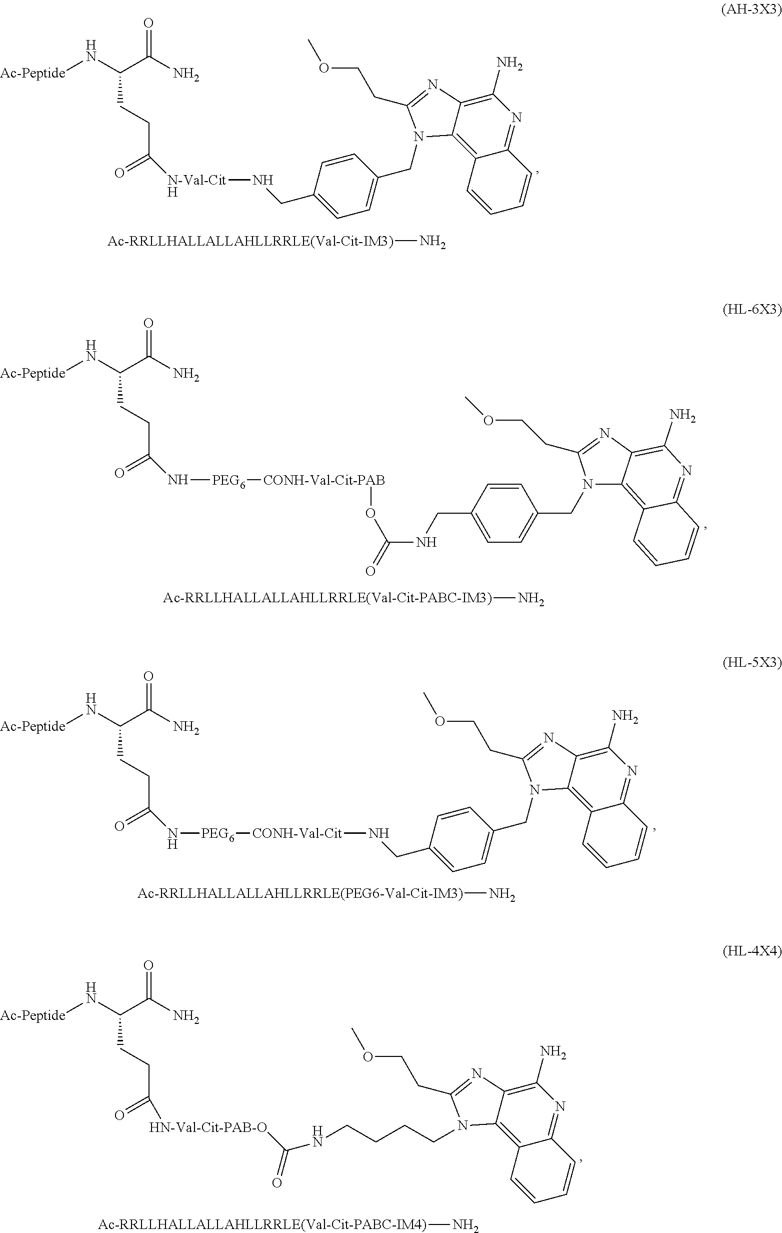 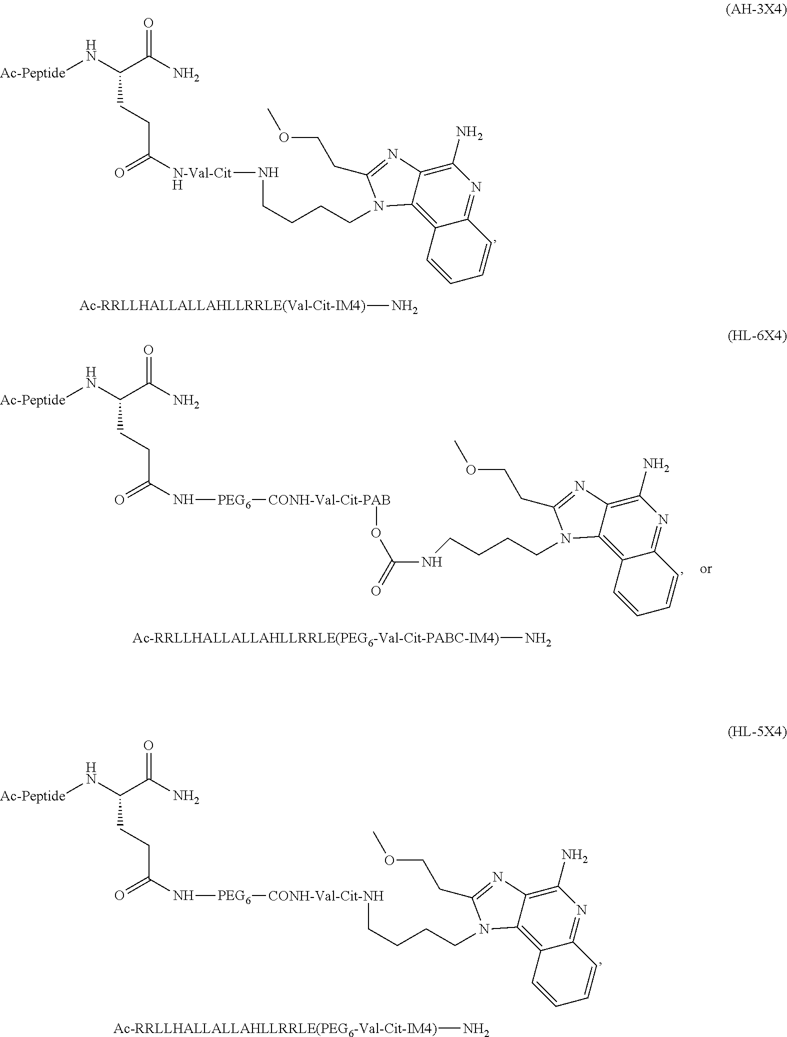
where Ac is Acetyl, Val is valine, Cit is Citrulline, PEG₆ is —NH—(CH₂)₂—(O—CH₂—CH₂)₆—CO, PABC is p-aminobenzyloxy carbonyl, PAB is p-aminobenzyloxy, and IMDQ is Formula (Ia) were R is —NH—; IM3 is Formula (Ik) where R is —NH—; IM4 is Formula (Ii) where R is —NH—.

10. (canceled)

11. A pharmaceutical composition comprising a compound of claim 1 and a pharmaceutical acceptable carrier or diluent.

12-19. (canceled)

20. A method for inducing a cell mediated immune response in a subject, wherein the method comprises:
locally administering a liquid form of the pharmaceutical composition comprising a compound of claim 1 and a pharmaceutical acceptable carrier or diluent into the subject, wherein in vivo physiological conditions reduce solubility of the DM component of the immunotherapy compound wherein the immunotherapy compounds form insoluble self-assemblies or aggregates in vivo;
whereby the insoluble self-assemblies or aggregates induce a cell mediated immune response at the local site of administration.

21. The method of claim 20, wherein the local site of administration is intratumoral or peritumoral.

22-24. (canceled)

25. The method of claim 20, further comprising administering at least one systemic checkpoint inhibitors.

26. The method of claim 25 wherein the inhibitor is an anti-PD-1 and/or anti-CTLA-4 antibody.

27. The method of claim 20, wherein the cell mediated immune response is an anti-tumor immune response
whereby the insoluble self-assemblies or aggregates induce an anti-tumor cell mediated immune response at the local site of administration and/or at a distant site from the site of administration of the pharmaceutical composition.

28-32. (canceled)

33. An immunotherapy compound having the structure of Formula (I):
DM-L-IM,
wherein DM comprises a peptide from about 18 to about 45 amino acids in length and comprises an amino acid sequence of RRLL(5)A(7)LAL(11)A(13)LLRRL (SEQ ID NO: 1) wherein amino acid positions (5), (7), (11) and (13) are each selected from A, L, or H;
wherein L is a linker connecting the IM to an amino group or carboxyl group of the peptide at the peptide termini or the lateral chain of an amino-acid such as lysine or glutamine; and,
IM is a toll-like receptor 7 (TLR7) and/or TLR8 agonist.

34. The compound of claim 33, wherein the peptide comprises an amino acid sequence selected from:
(SEQ ID NO: 2)
RRLLHAHLALHAHLLRRLK;
(SEQ ID NO: 3)
RRLLAAHLALHAALLRRLK;
(SEQ ID NO: 4)
RRLLHALLALLAHLLRRLK;
(SEQ ID NO: 6)
KRRLLHALLALLAHLLRRLK;
(SEQ ID NO: 7)
KRRLLAAHLALHAALLRRLK;
or
(SEQ ID NO: 8)
RRLLHALLALLAHLLRRLE

35. The compound of claim 33, wherein the linker L is -[A1]—NH—, and A1 is selected from:
-A2-A3-(CH₂)ₓ—CO—,
A2-A3-CH₂—O—CH₂—CO—,
A2-A3-(CH₂)ₓ—O—(CH₂)ₓ—O—(CH₂)ₓ—O—(CH₂)ₓ—CO—,
A2-Valine-Alanine-A4-,
A2-Valine-Citrulline-A4-,
A2-Glutamate-Valine-Citrulline-A4-, or
A2-Phenylalanine-Lysine-A4-;
A2 is selected from:
-A5-(CH₂)ₓ-A6-, -A5-(CH₂)ₓ—O—(CH₂)ₓ—O—(CH₂)ₓ—O—(CH₂)ₓ-A3-,
A5-(CH₂)₂—(O—CH₂—CH₂)ₓ-A6-
A5-CH₂—O—CH₂-A6-,
A5-(CH₂)ₓ-A6-,
A5-(CH₂)ₓ—O—(CH₂)ₓ—O—(CH₂)ₓ—O—(CH₂)ₓ-A6-, or,
A5-NH—(CH₂)₂—O—(CH₂)-A6-;
A3 is —CO— or —NH—;
A4 is p-aminobenzyloxy carbonyl (PABC): 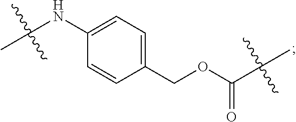 [SMILES: *Nc1ccc(COC(*)=O)cc1]
or nothing;
A5 and A6 are —CO— or —NH—, one or more natural or non-natural amino-acids, or nothing; and,
x is any integer from 1 to 12, or wherein x is any integer from 2 to 12.

36. The compound of claim 33, wherein the IM is selected from Formula (Ia), (Ib), (Ic), (Id), (Ie), (If), (Ig), (Ih), (Ii), (Ij), (Ik), (Il) or (Im), wherein:
R₂ is selected from:
CH₃, —CH₂CH₃, —CH₂CH₂CH₃, —CH₂CH(CH₃)₂, —CH₂CH₂CH₂CH₃, —CH₂CH₂CH₂CH₂CH₃, CH₂OCH₂CH₃, —CH₂CH₂OCH₃, —CH₂NHCH₂CH₃, and —CH₂Ph; and,
R comprises the linker;
B is selected from 0 and NH; and,
m is any integer from 1 to 11.

37. (canceled)

38. An immunostimulatory compound having the structure of Formula (I): DM-L-IM, wherein DM comprises a peptide, L is a linker and IM is a toll-like receptor 7 (TLR7) and/or TLR8 agonist, wherein the compounds are selected from: 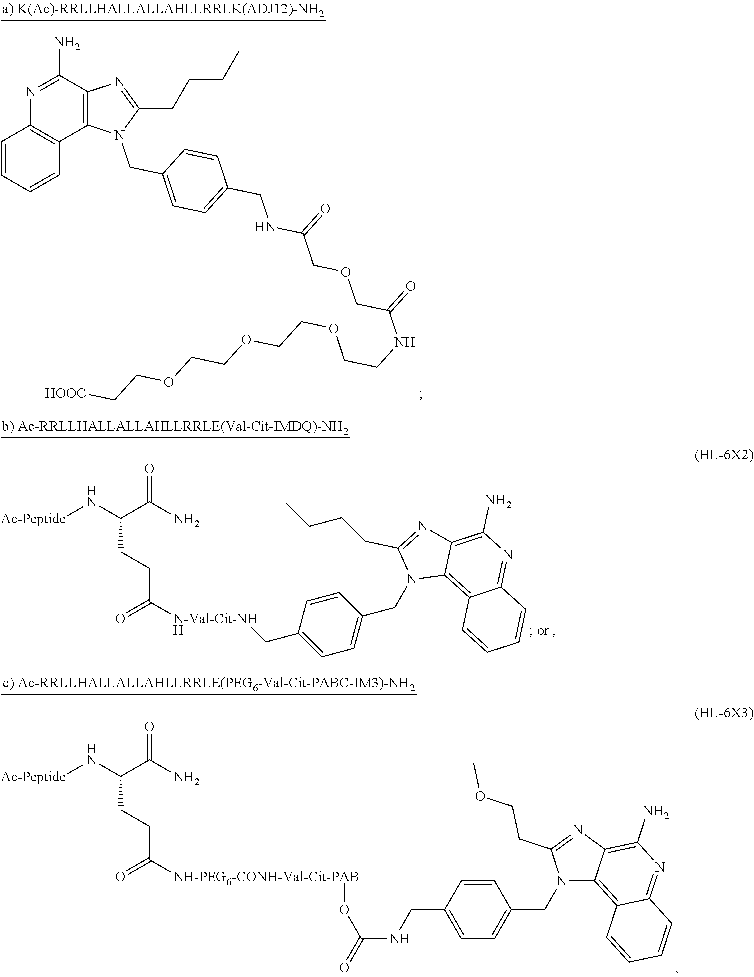
(named kHL-12) ADJ12 is derived from Formula I(a) where R of the IM is —NH—CO—CH₂—O—CH₂—CO—NH—((CH₂)₂O)₃—(CH₂)₂—COOH, or is
wherein Ac is acetyl; Val is valine, Cit is Citrulline, PEG₆ is —NH—(CH₂)₂—(O—CH₂—CH₂)₆—CO, PABC is p-aminobenzyloxy carbonyl, PAB is p-aminobenzyloxy, and IMDQ is Formula (Ia) were R is —NH—; and IM3 is Formula (Ik) where R is —NH—.

39-45. (canceled)

46. A method for inducing a cell mediated immune response in a subject, wherein the method comprises:
locally administering a liquid form of the pharmaceutical composition comprising a compound of claim 33 and a pharmaceutical acceptable carrier or diluent into the subject, wherein in vivo physiological conditions reduce solubility of the DM component of the immunotherapy compound wherein the immunotherapy compounds form insoluble self-assemblies or aggregates in vivo;
whereby the insoluble self-assemblies or aggregates induce a cell mediated immune response at the local site of administration.

47-58. (canceled)


SEQUENCE LISTING (8 sequences)

SEQ ID NO: 1 - protein, 18 aa, artificial sequence
  peptide
  MOD_RES (5)..(5): Ala, Leu or His
  MOD_RES (7)..(7): Ala, Leu or His
  MOD_RES (11)..(11): Ala, Leu or His
  MOD_RES (13)..(13): Ala, Leu or His
RRLLXAXLALXAXLLRRL

SEQ ID NO: 2 - protein, 19 aa, artificial sequence
  peptide
RRLLHAHLALHAHLLRRLK

SEQ ID NO: 3 - protein, 19 aa, artificial sequence
  peptide
RRLLAAHLALHAALLRRLK

SEQ ID NO: 4 - protein, 19 aa, artificial sequence
  peptide
RRLLHALLALLAHLLRRLK

SEQ ID NO: 5 - protein, 19 aa, artificial sequence
  peptide
RRLLKAKLALKAKLLRRLK

SEQ ID NO: 6 - protein, 20 aa, artificial sequence
  peptide
KRRLLHALLALLAHLLRRLK

SEQ ID NO: 7 - protein, 20 aa, artificial sequence
  peptide
KRRLLAAHLALHAALLRRLK

SEQ ID NO: 8 - protein, 19 aa, artificial sequence
  peptide
RRLLHALLALLAHLLRRLE